SEQUENCE LISTING (46 sequences)

SEQ ID NO: 1 - DNA, 531 bp, Rhipicephalus appendiculatus
  full length lipocalin Japanin
atgaaggtcctgcgatgtcttgtttgctcattttatataattgtatctttgataaccaccatgacaatcggcacccccagcatgccggcaatcaacacacagactctctaccttgcgggacactcatcgaagctgttcgaacgtaatgtggggtgcgtcaaaacaagatacttaaaccaaacaggagattgggtgacgagaagtctgatttacgtattcacctttgacacagaaccttgggtaacacaggccggtgcttttcaagtaaagtgggaaccatattctccactgctgcgtgtgaaggcttcagattacgtgagagataatttgggggcgaagccggactactttatacggacatacgacaacgactttttacttctatcagatttgaaagaggtacgatcaacatgctcgctctgggtgacactaaagtatgtggacagaattccagagactataaatagaacattttatacgatctgcccagatcctgtccccgttccttttgacgagaggtgctatccataa

SEQ ID NO: 2 - protein, 176 aa, Rhipicephalus appendiculatus
  full length lipocalin Japanin
MKVLRCLVCSFYIIVSLITTMTIGTPSMPAINTQTLYLAGHSSKLFERNVGCVKTRYLNQTGDWVTRSLIYVFTFDTEPWVTQAGAFQVKWEPYSPLLRVKASDYVRDNLGAKPDYFIRTYDNDFLLLSDLKEVRSTCSLWVTLKYVDRIPETINRTFYTICPDPVPVPFDERCYP

SEQ ID NO: 3 - DNA, 570 bp, Dermacentor andersoni
  Dermacentor andersoni E1244 Japanin homologue
atgaaactaaacttttttgtgattcttgctgtgtacattagcgtgtttgccatgacttacgttgaaacatctaacgaatcacctccgttttatgagttacctgcgaactctactgataccttgtatcttgttggtcattcagtcgatctgtttcgctggatgttaacatgcgttagaacgaactatacttctcgacaaggcgaagtggtaaaccgcacactgatttttaactactcgaaagagtttaacgagactccagaagagatgagctttccttttcaagtgacggtgccagatgtaccgataatgtttgacttggagttgaatgtgaccgactcgttagtaaactatactggggcacaatccacgtaccatattatctactataacgacgaaagcatggtattgggagacaaaatgcccacagttagtgaaagggccatttgtagcttgtgggtcaaagaaaactttacgctggaacaccaaatcccctttatggctaacttgagttttcacacctcttgcaaaaacgcgttgtattatggctacctagacacgtgttcgaaataa

SEQ ID NO: 4 - protein, 172 aa, Dermacentor andersoni
  Dermacentor andersoni E1244 Japanin homologue
MTYVETSNESPPFYELPANSTDTLYLVGHSVDLFRWMLTCVRTNYTSRQGEVVNRTLIFNYSKEFNETPEEMSFPFQVTVPDVPIMFDLELNVTDSLVNYTGAQSTYHIIYYNDESMVLGDKMPTVSERAICSLWVKENFTLEHQIPFMANLSFHTSCKNALYYGYLDTCSK

SEQ ID NO: 5 - DNA, 570 bp, Rhipicephalus microplus
  Rhipicephalus microplus RM-CK185494 Japanin homologue
ggtgaaaagccaggcccttcttatccgcaggaaaaatagaggcccgagaatggagccctgcggaacaccagatgccaccggagctgaaggagaagctgtgccatcaatgactacacattgccgccttgacgagaggactctcaacaagtcagagtactcatgcgtggcttctagctacaaggaaattgggggaggatttatccaccgcacacttctcctgaaaagggcaccgacaccaggagagtactgggacacgcaaataccccttcaaatacaagctaccatgtgctatatgacaatgaacttgactttatcctgtccgctgataaagctcggtgcaaaggaacagtatctgatgtttcgtcagacgtggaattacatgctgctgaccgaattgatagagttaaaggaacgaccactgtgttctatttgggcgaagaaggactttgcaagaaaaagtaaggtggacgaatatacattgacattatttcacgcaatttgcaaagatgcagtctacgacgggtacccagagtattgcccgatacttgtgaaataaaacccatttccttt

SEQ ID NO: 6 - protein, 168 aa, Rhipicephalus microplus
  Rhipicephalus microplus RM-CK185494 Japanin homologue
MEPCGTPDATGAEGEAVPSMTTHCRLDERTLNKSEYSCVASSYKEIGGGFIHRTLLLKRAPTPGEYWDTQIPLQIQATMCYMTMNLTLSCPLIKLGAKEQYLMFRQTWNYMLLTELIELKERPLCSIWAKKDFARKSKVDEYTLTLFHAICKDAVYDGYPEYCPILVK

SEQ ID NO: 7 - DNA, 696 bp, Amblyomma americanum
  Amblyomma americanum CX766068 Japanin homologue
gggccctggaaaagtgtctccaaaatgcaaaggcacggataaatgcctgtgttatgatgggtatttccgcaagacagatacagaatgcgtccttctgaaaagagactgcctggatagaaaatatgattcggacgggcttttggcagcgacggaagacatctacatggtgaaaagttcggaaacgctgaaaatggattttcccacaaaatgtctcagatctgcattcctatcgtcgaccggtaatgtaattcgtcgcacaatccagtacagagagaacattggagaacctcaggacgtgtggctgacaaagaatttcactgttatacttaaaaaagacaaagacacactgacggttcaaaacactgaaacactgaaccgtttgacccgggcaggtaaatctcagtacaggattgtgcattctggtggtgactgtttgatattgggagaccaaattccccttggaggacaaacaacgacatgcactatgtgggtcaagcagagctctattggacagacgttttctgaggagtgcgaattcttttttaaacactactgcgccacatcgagcttgactgtgaaagatacacggtgtgactaacacttgagcgcccaggcacgccaccgaatctgctttcttaccaccaaggaagacaatagcaggctgcattcattgcgttccatccatctcttatatgg

SEQ ID NO: 8 - protein, 144 aa, Amblyomma americanum
  Amblyomma americanum CX766068 Japanin homologue
MVKSSETLKMDFPTKCLRSAFLSSTGNVIRRTIQYRENIGEPQDVWLTKNFTVILKKDKDTLTVQNTETLNRLTRAGKSQYRIVHSGGDCLILGDQIPLGGQTTTCTMWVKQSSIGQTFSEECEFFFKHYCATSSLTVKDTRCD

SEQ ID NO: 9 - DNA, 748 bp, Rhipicephalus appendiculatus
  Rhipicephalus appendiculatus CD796501 Japanin homologue
atggcaactttgacagctcttatattttcggtgcttgccgtaccaataacttccattgatattcaccaatttgtccaagacacaattctgacaccttatcaagatccttggaagtttattgcaaatcacagcactgtttacctctcacgtgtatccatagcaccttcgcttgaagataaaccacaatttccttgtgtacgttcacggtattggagtaaatcaggagaaagcgtgaaccggtcactcgacatttataatacaacgaacgaaggggattacaggtccacaaacattgatcttaaggtaaaacacgagaaaagccgcacaattcttgatgttgacgtggaagggggaacatcttctataatgagcattccttatatgaagacatctagcgcgtcgaatctaacaggatacacattccttgtactgtatagtgaccataattgtcttattctagccgaaacgcttcaaacagcaacaataaaaaaaattaccttcgctgctggatgtggcttccacaaagtcgtatacaaaagctacagaaatgttgccagttcatttacacactcctgtgcctacgtgccgctgaagatgttgaagtatttcagccctcatgccttactaatacgaccaaagcaagccattgatctggcggagatcctgggacaccgcctggtaactgatatgggattttttttgctgttttatgatgtttccgtccatgaataaagattttttgtgaaag

SEQ ID NO: 10 - protein, 225 aa, Rhipicephalus appendiculatus
  Rhipicephalus appendiculatus CD796501 Japanin homologue
IDIHQFVQDTILTPYQDPWKFIANHSTVYLSRVSIAPSLEDKPQFPCVRSRYWSKSGESVNRSLDIYNTTNEGDYRSTNIDLKVKHEKSRTILDVDVEGGTSSIMSIPYMKTSSASNLTGYTFLVLYSDHNCLILAETLQTATIKKITFAAGCGFHKVVYKSYRNVASSFTHSCAYVPLKMLKYFSPHALLIRPKQAIDLAEILGHRLVTDMGFFLLFYDVSVHE

SEQ ID NO: 11 - DNA, 726 bp, Rhipicephalus microplus
  Rhipicephalus microplus CV436349 Japanin homologue
taaaactggcagttctgttcaccgcaggatctcttggatcgcgtgtcagcaaccacagctacagaaaatggaagcgaatcgcatctactgggccgtgctatttgtcctgtgcaactgtgcagtagtcttcagccacttggctcctcccgcgggaccaagaagactgcaccacgatgtggtggacagcttcaaggtaatgcagagcttcccctatgccgtggcggtcgtcgactctgacaacgacacatacttcaactgctggacagtggagcgtaaggcgatcgacgatgtcgcaagaacggccacatacgagggacattttccgtcggctggacgagtcgtaccgttctatgtgaaagccgaagagaactccccgaagttcaccttcacccttgacgatgatccgactccgaaagaaggcatgttctactacaccgatgacgaaaactgtgtcgtcgaggaccttgagtaccacaagcatcagtgcgtcctttgggtcaagaaagagctggagaactcagtgcctcagagctgcattaactactatgtcgatatatgtggcgttggtgttcccatacacagcagagacctttgcagagaccagtgacgtgactgctgaagtaacgcgcaatccttctcagtgcttcagagtgaaaaggttgtgttagtaaaaatttacttgctgacaactgtggtctcgtgaacaataaatttcctttcatactg

SEQ ID NO: 12 - protein, 201 aa, Rhipicephalus microplus
  Rhipicephalus microplus CV436349 Japanin homologue
KTGSSVHRRISWIACQQPQLQKMEANRIYWAVLFVLCNCAVVFSHLAPPAGPRRLHHDVVDSFKVMQSFPYAVAVVDSDNDTYFNCWTVERKAIDDVARTATYEGHFPSAGRVVPFYVKAEENSPKFTFTLDDDPTPKEGMFYYTDDENCVVEDLEYHKHQCVLWVKKELENSVPQSCINYYVDICGVGVPIHSRDLCRDQ

SEQ ID NO: 13 - DNA, 21 bp, Artificial Sequence
  synthetic PCR amplification degenerate forward primer External Primer 1
atgccngcnatcaayacncaa

SEQ ID NO: 14 - DNA, 21 bp, Artificial Sequence
  synthetic PCR amplification degenerate forward primer External Primer 2
atgccngcnatcaayacncag

SEQ ID NO: 15 - DNA, 21 bp, Artificial Sequence
  synthetic PCR amplification degenerate forward primer External Primer 3
atgccngcnatwaayacncaa

SEQ ID NO: 16 - DNA, 21 bp, Artificial Sequence
  synthetic PCR amplification degenerate forward primer External Primer 4
atgccngcnatwaayacncag

SEQ ID NO: 17 - DNA, 23 bp, Artificial Sequence
  synthetic nested PCR amplification Internal Primer 1
gcyacncagacnytntayctngc

SEQ ID NO: 18 - DNA, 23 bp, Artificial Sequence
  synthetic nested PCR amplification Internal Primer 2
gcyacncagacnytntayttrgc

SEQ ID NO: 19 - DNA, 26 bp, Artificial Sequence
  synthetic 1st round nested PCR Forward primer 5
tggcattctttgaagctctgtcatca

SEQ ID NO: 20 - DNA, 31 bp, Artificial Sequence
  synthetic 1st round nested PCR Reverse primer 3
gctttttattttccgttatggatagcacctc

SEQ ID NO: 21 - DNA, 28 bp, Artificial Sequence
  synthetic 2nd round nested PCR Forward primer 6
cgttaggatccggcattctttgaagctc

SEQ ID NO: 22 - DNA, 27 bp, Artificial Sequence
  synthetic 2nd round nested PCR Reverse primer 4
gtttagcggccgccgttatggatagca

SEQ ID NO: 23 - DNA, 28 bp, Artificial Sequence
  synthetic three-stage nested PCR Forward primer 7
cgttaggatccggcattctttgaagctc

SEQ ID NO: 24 - DNA, 31 bp, Artificial Sequence
  synthetic first stage nested PCR Reverse primer 5
atgagagcctccttgtggatagcacctctcg

SEQ ID NO: 25 - DNA, 33 bp, Artificial Sequence
  synthetic second stage nested PCR Reverse primer 6
ttagtgatgatgatgatgatgagagcctccttg

SEQ ID NO: 26 - DNA, 27 bp, Artificial Sequence
  synthetic third stage nested PCR Reverse primer 7
aagtgcggccgcttagtgatgatgatg

SEQ ID NO: 27 - DNA, 33 bp, Artificial Sequence
  synthetic PCR amplification Forward primer 8
gcaggcataggatccaaaatgaaactaaacttt

SEQ ID NO: 28 - DNA, 27 bp, Artificial Sequence
  synthetic PCR amplification Reverse primer 8
tattgcggccgcttatttcgaacacgt

SEQ ID NO: 29 - DNA, 20 bp, Artificial Sequence
  synthetic gene-specific reverse transcription primer GSP1B
gttatggatagcacctctcg

SEQ ID NO: 30 - DNA, 24 bp, Artificial Sequence
  synthetic first round nested PCR amplification primer GSP2A
agccttcacacgcagcagtggaga

SEQ ID NO: 31 - DNA, 22 bp, Artificial Sequence
  synthetic second round nested PCR amplification primer GSP3
gcctgtgttacccaaggttctg

SEQ ID NO: 32 - DNA, 774 bp, Artificial Sequence
  synthetic degenerate sequence encoding Dermacentor andersoni Japanin
gagacagcggtgtgcccttagcgttaggtgccagtggttctaggttcagcctactattgtgcacgttaattcgagactatataaagagtgtacgaaagccgcctcacaacaggaagctaaatccattgcgggagtgatcttcagttgggaaaggaaatgaagctcaatttttttgttattctcgccgtatatatctccgtctttgccatgacatatgttgaaacgtccaacgaatcacccccattttatgagctgcctgccaacagcaccgataccctttacctcgtggggcactctgtcgatctattcagatggatgctaacatgcgtaagaacaaactacaccagtcggcaaggtgaagtggtcaatcggactctgattttcaactattcaaaagagttcaacgaaactccggaagaaatgagctttccatttcaagtgacagtaccagacgttccgataatgtttgaccttgaactgaatgtgacagactccctggtaaactacactggagcacaatcaacgtaccacataatctattacaatgatgagtctatggttctcggggacaagatgccaaccgtatcggaacgagcgatatgttcactctgggtgaaggagaacttcactctagaacaccaaatccctttcatggcaaacttgtcttttcatacaagctgcaaaaatgcactatactatggatatctcgatacctgcagcaagtagtaggacgcccgtggaacacgaataaatttttctgacagaaaccgtaaa

SEQ ID NO: 33 - DNA, 460 bp, Artificial Sequence
  synthetic consensus sequence for 3′ region of Japanin
actctctaccttgcgggacactcatcgaagctgttcgaacgtaatgtggggtgcgtcaaaacaagatacttaaaccaaacaggagattgggtgacgagaagtctgatttacgtattcacctttgncacagaaccttgggtaacacaggccggtgcttttcaagtaaagtgggaaccatattctccactgctgcgtgtgaaggcttcagattacgtnagagataatttgggggcgaagccggactactttatacggacatacgacaacgacnttttacttctatcagatttgaaagaggtacgatcaacatgctcgctctgggtgacactaaagtatgtggacagaattccagagactataaatagaacattttatacgatctgcccagatcctgtccccgttccttttgacgagaggtgctatccataacggaaaataaaaagctttttgaatttttgca

SEQ ID NO: 34 - DNA, 439 bp, Rhipicephalus appendiculatus
  5′ cDNA sequence of Japanin
ttgtaagagcgtacttccactttyttaggaagagggcaaggactcgtggctgaaagaccgcaaaaccattcttgcgagagttattgacaatttcaagttgatttgctaaacgaatcaagtttctctcaccggagtgccaaatcgtaccaatattgtataaaggcgctgcgaaaaaggatttccagtacacattggcattctttgaagctctgtcatcattttcagaaaaaaatgaaggtcctgcgatgtcttgtttgctcattttatataattgtatctttgataaccaccatgacaatcggcacccccagcatgccggcaatcaacacacagactctctaccttgcgggacactcatcgaagctgttcgaacgtaatgtggggtgcgtcaaaacaagatacttaaaccaaacaggagattgggtgacgagaagtctga

SEQ ID NO: 35 - DNA, 822 bp, Rhipicephalus appendiculatus
  full length Japanin cDNA
aaagtgcgttgtaagagcgtacttccactttyttaggaagagggcaaggactcgtggctgaaagaccgcaaaaccattcttgcgagagttattgacaatttcaagttgatttgctaaacgaatcaagtttctctcaccggagtgccaaatcgtaccaatattgtataaaggcgctgcgaaaaaggatttccagtacacattggcattctttgaagctctgtcatcattttcagaaaaaaatgaaggtcctgcgatgtcttgtttgctcattttatataattgtatctttgataaccaccatgacaatcggcacccccagcatgccggcaatcaacacacagactctctaccttgcgggacactcatcgaagctgttcgaacgtaatgtggggtgcgtcaaaacaagatacttaaaccaaacaggagattgggtgacgagaagtctgatttacgtattcacctttgacacagaaccttgggtaacacaggccggtgcttttcaagtaaagtgggaaccatattctccactgctgcgtgtgaaggcttcagattacgtnagagataatttgggggcgaagccggactactttatacggacatacgacaacgacnttttacttctatcagatttgaaagaggtacgatcaacatgctcgctctgggtgacactaaagtatgtggacagaattccagagactataaatagaacattttatacgatctgcccagatcctgtccccgttccttttgacgagaggtgctatccataacggaaaataaaaagctttttgaatttttgcaaaaaaaaaaaaaaaaaaaaaa

SEQ ID NO: 36 - protein, 30 aa, Artificial Sequence
  synthetic N-terminus of Japanin with potential cleavage site
MKVLRCLVCSFYIIVSLITTMTIGTPSMPA

SEQ ID NO: 37 - protein, 152 aa, Rhipicephalus appendiculatus
  mature Japanin
TPSMPAINTQTLYLAGHSSKLFERNVGCVKTRYLNQTGDWVTRSLIYVFTFDTEPWVTQAGAFQVKWEPYSPLLRVKASDYVRDNLGAKPDYFIRTYDNDFLLLSDLKEVRSTCSLWVTLKYVDRIPETINRTFYTICPDPVPVPFDERCYP

SEQ ID NO: 38 - protein, 223 aa, Rhipicephalus appendiculatus
  Rhipicephalus appendiculatus CD796501 Japanin homologue
IDIHQFVQDTILTPYQDPWKFIANHSTVYLSRVSIAPSLEDKPQFPCVRSRYWSKSGESVNRSLDIYNTTNEGDYRSTNIDLKVKHEKSRTILDVDVEGGTSSIMSIPYMKTSSASNLTGYTFLVLYSDHNCLILAETLQTATIKKITFAAGCGFHKVVYKSYRNVASSFTHSCAYVPLKMLKYFSPHALLIRPKQAIDLAEILGHRLVTDMGFFLLFYDVSV

SEQ ID NO: 39 - protein, 7 aa, Artificial Sequence
  synthetic N-terminus of Japanin for degenerate external primer design
MPAINTQ

SEQ ID NO: 40 - DNA, 15 bp, Artificial Sequence
  synthetic oligo(dT)-15 reverse transcription primer
ttttttttttttttt

SEQ ID NO: 41 - DNA, 21 bp, Artificial Sequence
  synthetic oligo(dT)-20-V reverse primer
ttttttttttttttttttttv

SEQ ID NO: 42 - protein, 8 aa, Artificial Sequence
  synthetic N-terminus of Japanin for internal primer design
NTQTLYLA

SEQ ID NO: 43 - protein, 15 aa, Artificial Sequence
  synthetic N-terminal Japanin sequence following signal sequence
TPSMPAINTQTLYLA

SEQ ID NO: 44 - protein, 6 aa, Artificial Sequence
  synthetic six residue polyhistidine fusion tag (His-tag) at C-terminus
HHHHHH

SEQ ID NO: 45 - protein, 4 aa, Artificial Sequence
  synthetic additional residues introduced between His-tag and native protein, TEV protease consensus cleavage site
QGGS

SEQ ID NO: 46 - protein, 5 aa, Artificial Sequence
  synthetic penta-His
HHHHH

(19) United States
(12) Patent Application Publication
(10) Pub. No.: US 2012/0070460 A1
(43) Pub. Date: Mar. 22, 2012

(54) DENDRITIC CELL MODULATORY MOLECULE

(75) Inventors: Jon Austyn, Oxford (GB); Stephen Preston, Oxford (GB); Patricia Nuttall, Oxfordshire (GB); Guido Paesen, Oxford (GB)

(73) Assignee: Natural Environment Research Council, Swindon (GB)

(21) Appl. No.: 13/119,180

(22) PCT Filed: Sep. 16, 2009
(86) PCT No.: PCT/GB2009/002219

§ 371 Date: Jun. 6, 2011

(30) Foreign Application Priority Data
Sep. 16, 2008 (GB) 08169765

(51) Int. Cl. A61K 39/35; C12N 15/12; C12N 15/63; C07K 16/18; C12N 1/21; C12N 1/19; C12N 5/10; C12N 5/0784; A61K 38/17; A61K 39/00; G01N 33/566; A61P 37/06; A61P 29/00; A61P 37/08; A61P 43/00; A61P 35/00; A61P 31/00; C07K 14/435

(52) U.S. Cl. 424/275.1; 530/350; 530/359; 536/23.5; 435/320.1; 530/387.9; 435/252.33; 435/254.2; 435/325; 435/348; 435/375; 424/184.1; 424/277.1; 435/7.21

(57) ABSTRACT

The present invention provides a dendritic cell modulatory molecule which modulates, and preferable inhibits, the differentiation and maturation of mammalian dendritic cells. The invention also provides pharmaceutical compositions comprising the dendritic cell modulatory molecule and homologues and active fragments thereof, antibodies thereto and methods of treatment and screening methods which utilise such molecules.

### FIELD OF THE INVENTION

The present invention relates to dendritic cell (DC) modulatory molecules. In particular, the invention relates to a molecule which modulates, and preferably inhibits, the differenitiation and maturation of mammalian DCs, particularly human DCs. Such molecules can be isolated from arthropod saliva, and more specifically from tick saliva. The invention also relates to the use of such molecules in therapy, and specifically to the use of such molecules in treating autoimmune disorders, allergies and other hypersensitivity reactions, transplant rejection and graft-versus-host disease, infectious diseases including those transmitted by ticks, cancers including haematological malignancies, and acute and chronic inflammatory diseases including inflammation associated with the aforementioned diseases.

### BACKGROUND TO THE INVENTION

The mammalian, and particularly the human, immune system is comprised of two arms, the innate and the adaptive immune systems. The cells of the innate immune system recognise, and respond to infectious agents in a generic manner. Although the innate immune system is a vital immediate barrier to infection, it does not confer specific, long-lasting protection against foreign entities, such as invading pathogens. In contrast, the cells of the adaptive immune system recognise specific foreign entities, and induce immunological memory to these specific entities in the host.

DCs interact with components of the innate immune system soon after infection by a pathogen and also form a central part of the mammalian adaptive immune response. DCs differentiate from precursor cells into immature DCs. Immature DCs are present throughout the body and, although other cells of the immune system also participate in this role, they are the major cell type responsible for initiation of adaptive immune responses, primarily through their capacity to trigger T cell activation.

Immature DCs constantly sample their surrounding environment for infectious agents such as viruses, bacteria and parasites, through pattern recognition receptors (PRRs) such as toll-like receptors (TLRs) which recognise specific chemical signals on the foreign entity, e.g. on a pathogen's surface. Once an entity such as a pathogen has been identified as foreign, the immature DC internalises the entity or fragments of it and degrades the protein and lipid antigens into peptides and glycopeptides or lipid fragments which are presented on the DC surface.

In response to foreign entity recognition, and/or other signals within the cell's environment (e.g. inflammatory cytokines), the immature DC undergoes several changes collectively termed ‘maturation’ and starts to develop into a mature DC. The maturing DC up-regulates expression of major histocompatability complex (MHC), and MHC-related molecules such as CD1, which bind the foreign entity-derived peptides and glycopeptides, and lipids, respectively, and allow them to be displayed on the DC surface. Simultaneously, the DC up-regulates expression of cell surface receptors known as costimulatory molecules including CD80, CD86 and CD40, which act as co-receptors for T lymphocyte activation. In addition, the DC begins to migrate to lymphoid tissues such as the lymph nodes and/or spleen, following chemotactic signals. Once in the lymphoid tissues, the DC activates T lymphocytes, by presenting them with the peptides and glycopeptides or lipid fragments derived from the foreign entity and delivering the appropriate co-stimulatory signals. Such activated T lymphocytes are responsible for propagating the adaptive immune response. The foreign entity may be a pathogen, an allergen or, in the case of an autoimmune response, a self-antigen incorrectly identified by the body as foreign.

As well as having a role in triggering T cell activation by antigen presentation and co-stimulation, mature DCs are involved in T cell regulation, such as polarisation of helper T cells into Th1, Th2, Th17 or regulatory (Treg) cells, the activation of cytotoxic T cells, and modulation of T cell homing, e.g. into the skin or gut and other mucosal sites.

The central role played by DCs in the adaptive immune response has led to interest in the modulation of DC function for therapeutic purposes, and there have been indications from animal models that DC modulators may be useful in the treatment of autoimmune and other inflammatory diseases (Subklewe et al. Human Immunology, 2007, 68(3), 147-155). It has also been suggested that DC modulators may be useful in the treatment of cancer. Clearly it would be advantageous to identify further molecules which act as DC modulators for therapeutic purposes.

Specifically, there remains a need for the identification of compounds with DC modulatory activity, and particularly with inhibitory activity, and the development of their use in the treatment of autoimmune and other inflammatory diseases, and in the treatment of cancer.

### SUMMARY OF THE INVENTION

Accordingly, the present invention provides an isolated DC modulatory molecule, wherein said molecule modulates, and preferably inhibits, mammalian DC differentiation and maturation.

In one embodiment, the isolated DC modulatory molecule modulates, and preferably inhibits, human DC differentiation and maturation. The DC modulatory molecules of the present invention may be isolated from arthropods, particularly from haematophagous arthropods. The isolated DC modulatory molecule may be a protein.

Haematophagous arthropods attach to their hosts, including mammals such as man, and feed for extended periods of time. The components that they deliver to the hosts, including components in saliva, can potentially induce host immune responses. Such responses may be deleterious to the arthropods and therefore the arthropods may need to suppress them. Given the central role of DC in triggering immunity, it may be advantageous to the arthropods to produce molecules that inhibit their function.

Haematophagous arthropods, and particularly ticks, may inhibit the host's immune system by inoculating the host with a variety of anti-inflammatory and immunomodulatory components (Ribeiro et al, Infectious Agents and Disease, 1992, 4(3), 143-152).

Several immunomodulatory molecules have been identified in tick saliva, including a homologue of macrophage migration inhibitory factor (MIF) (Jaworski et al, Insect Molecular Biology, 2001, 10(4), 323-331), a homologue of leukocyte elastase inhibitor which is secreted by human macrophages, monocytes and neutrophils (Leboulle et al, The Journal of Biological Chemistry, 2002, 277(12), 10083-10089), glycosylated protein p36, which is thought to suppress mitogen driven in vitro proliferation of murine spleen cells (Bergman et al, Journal of Parasitology, 2000, 86, 516-525), B cell inhibitory protein (BIP) (Hannier et al, Immunology, 2004, 113, 401-408), and B cell inhibitory factor (BIF) (Yu et al, Biochemical and Biophysical Research Communications, 2006, 343, 585-590). However, many of these molecules do not have a defined cellular target, and none of these molecules have been identified as having an inhibitory effect on both the differentiation and maturation of mammalian DCs and in particular of human DCs.

Salp15 is a protein present in tick saliva which has been found to act on immature human DCs (Anguita et al, Immunity, 2002, 16, 849-859 and Hovius et al, Vector borne and Zoonotic diseases, 2007, 7(3), 296-302). However, assays involving the incubation of immature human DCs with Salp15 in the presence of an immunomodulatory stimulus have shown that Salp15 does not inhibit the upregulation of costimulatory molecules (e.g. CD86). Salp15 does not therefore inhibit the maturation of human DCs.

Prostaglandin E₂ (PGE₂) is a non-protein molecule present in tick saliva that may modulate the activity of immature murine DCs, but has a minimal effect on maturation of these murine DCs (Sa-Nunes et al, The Journal of Immunology, 2007, 179, 1497-1505). PGE₂ is capable of enhancing the maturation of human DCs but there is no evidence that it can act to inhibit the differentiation and maturation of human DCs.

It has also been suggested that tick saliva and salivary gland extract (SGE) may possess the ability to modulate the differentiation and maturation of murine DCs (Cavassani et al, Immunology, 2005, 114, 235-245, and Skallova et al, Journal of Immunology, 2008, 180, 6186-6192). However, the molecules responsible for these activities have not been isolated and, to date, there is no evidence that tick saliva has the ability to inhibit both the differentiation and maturation of human DCs.

Surprisingly, the inventors have now isolated a molecule that modulates, and preferably inhibits, both the differentiation and maturation of mammalian DCs, in particular human DCs.

The term “isolated” is intended to convey that the molecule is no longer within its natural environment. This term includes molecules which have been removed from their natural environment, and molecules which are identical to these but have been produced synthetically. Isolated molecules of the invention are generally substantially pure. By “substantially pure” is meant that the composition comprises at least about 50% of the molecule of interest. In some embodiments the composition may comprise at least about 60%, at least about 70%, at least about 80%, at least about 90%, at least about 95%, at least about 99% or more of the molecule. Put another way, the composition may comprise less than about 50% of other molecules. In other embodiments, the composition may comprise less than about 40%, less than about 30%, less than about 20%, less than about 10%, less than about 5%, less than about 1% or less of other molecules.

Activities of the Molecules of the Invention:

The molecules of the invention “modulate”, i.e alter, or “inhibit”, i.e. reduce, both the differentiation and maturation of mammalian DCs. In one embodiment, these may be human DCs. Suitable assays for assessing modulation or inhibition of DC differentiation and maturation are described below. It will be apparent to the skilled person that the markers described here for the assessment of modulation or inhibition of differentiation and maturation are provided by way of example only, and are not intended to be limiting. In one embodiment, the molecule of the invention reduces both DC differentiation and maturation by at least 20% as measured, for example, by the assays discussed below. In further embodiments, the inhibition of both DC differentiation and maturation may be 30%, 40%, 50%, 60%, 70%, 80%, 90% or more.

By “DC differentiation” is meant the development of a cell precursor, such as a bone marrow derived progenitor or a blood monocyte, into an immature DC. Modulation, for example inhibition of DC differentiation can be assessed phenotypically and, ultimately functionally, using standard assays known in the art.

Inhibition of phenotypic differentiation of precursors into immature DCs can be assessed using cellular markers whose expression is altered as the precursor cell differentiates into an immature DC. For example, as described in FIG. 23 and the accompanying description, monocytes are DC precursors which are CD14-positive and CD1-negative. Immature DCs are CD14-negative and CD1-positive. Hence, differentiation of monocytes into immature DCs may be detected by a decrease in CD14 and an increase in CD1. Inhibition of differentiation of precursor cells into immature DCs by the molecules of the invention may be detected by the continued presence of precursor cells that are CD14-positive and CD-1 negative.

Functional differentiation of precursor cells and developed DCs can be assessed using any assay which distinguishes between precursor cells and differentiated DCs based on their activities. For example, unlike precursor cells, developed DCs, particularly after stimulation as described below, are capable of triggering T cell proliferation in an in vitro assay. Typical T cell proliferation assays include the allogeneic mixed leukocyte reaction (MLR) and oxidative mitogenesis.

By “DC maturation” is meant the process which occurs after a precursor cell has differentiated into an immature DC. Specifically, this term relates to the changes which occur when a differentiated, immature DC encounters a stimulus, and is converted into a mature DC. The stimulus may be a component of an infectious agent such as a pathogen, which is sensed via PRR such as TLR, certain cytokines which act through cytokine receptors, and/or specialised cell surface molecules of other cell types such as CD154 of activated T cells. The changes associated with maturation of immature DCs typically include the up-regulation of expression of costimulatory molecules e.g. CD80 and CD86 and the presentation of antigens from the pathogenic-derived component on the DC's surface, typically as peptide-MHC and lipid-CD1 complexes. Maturation of immature DCs may also be associated with migration of the DC to the secondary lymphoid tissues.

The molecules of the invention may act to modulate or inhibit any of these changes associated with maturation of immature DCs. The ability of the molecules of the invention to inhibit immature DC maturation may thus be assessed by their ability to decrease the expression of CD86 and/or CD80 and/or MHC molecules. The ability of the molecules of the invention to inhibit immature DC maturation may be assessed by their ability to decrease the expression and/or the secretion of TNFα. The ability of the molecules of the invention to inhibit DC maturation may optionally be assessed following poly(I:C), LPS or IFNγ stimulation. The ability of the molecules of the invention to inhibit DC maturation may optionally be assessed following CD40L, IFNα, or a TLR7 or TLR8 ligand (e.g. CL097) stimulation. Experiments for assessing inhibition of maturation of immature DC are described in the examples. Further methods for assessing the inhibition of immature DC maturation will be known to a person skilled in the art.

As described above, the molecules of the invention act to modulate, and preferably inhibit, differentiation of precursor cells into immature DCs and to modulate, and preferably inhibit, the subsequent maturation of immature DCs into mature DCs. Such modulation or inhibition of both DC differentiation and maturation is likely to have downstream modulatory effects on the immune system as a whole, as described in more detail below.

Prior to activation by an antigen presenting cell, T lymphocytes are referred to as “naïve”. Each T lymphocyte is specific for a particular antigen, and can only be activated by a ‘specialised’ antigen presenting cell, such as a DC, which is presenting this cognate antigen. Conventional T lymphocytes recognise the antigen-MHC complex through the T cell receptor (TCR), which is a heterodimeric structure, comprising α and β chains. However, signalling through the TCR, in the absence of costimulation, results in a state of antigen-unresponsiveness or anergy, or abortive activation and cell death. Therefore, the costimulatory molecules, which are upregulated on the surface of immature DCs during the maturation process, and are recognised by receptor molecules such as CD28 (in the case of CD80 and CD86) or CD154 (for CD40) on the T lymphocyte's surface, are vital for the activation of T lymphocytes.

In one aspect of the invention, the inhibition of DC differentiation and maturation afforded by the molecules described above, results in a decrease in T lymphocyte activation. By “T lymphocyte activation” is meant activation of helper T cells, including Th1, Th2, Th17 or Treg cells, and optionally the activation of cytotoxic T cells which is often dependent on prior activation of helper T cells.

A decrease in T cell activation may be assessed by methods known in the art. By way of example, and not limitation, T cell activation may be assessed by in vitro assays of cytokine secretion [e.g. interleukin (IL)-2 production] or T cell proliferation triggered by DC (e.g. allogeneic MLR or oxidative mitogenesis) or by in vivo assays of T cell responses to model antigens (e.g. ovalbumin) in normal or transgenic animals using similar assays of T cells isolated ex vivo before and after antigen re-stimulation.

In one embodiment, the molecules of the invention will reduce T lymphocyte activation by at least about 20% compared to a standard assay in the absence of a DC differentiation and maturation inhibiting molecule. In further embodiments, the inhibition of DC differentiation and maturation may reduce T lymphocyte activation by at least about 30%, 40%, 50%, 60%, 70%, 80%, 90% or more.

In one aspect of the invention, the modulation or inhibition of DC differentiation and maturation afforded by the molecules described above, results in a modulation in T lymphocyte regulation. In particular, the molecules of the invention may modulate the polarisation of T lymphocytes into Th1 versus Th2 versus Th17 versus regulatory T (Treg) or follicular helper T (Thf) cells. Modulation of T lymphocyte polarisation by the molecules of the invention may be assessed by measuring T-lymphocyte-derived cytokines typically associated with different types of CD4 and CD8 T lymphocytes in in vitro assays. For Th1 cells, these include IFN-γ; for Th2 cells, IL-4, IL-5, and IL-13; for Th17 cells, IL-17; and for Treg cells, IL-10 and TGFβ. The respective types of CD4 cell can also be assayed by measuring expression of T-bet, GATA-3, ROR-γ-t and FoxP3 respectively, or by measuring expression of Bc16 for Thf cells. Alternatively, the phenotype of the different cells can be assessed phenotypically, e.g. by assessing the chemokine receptors and other phenotypic markers that they express.

T lymphocytes are one of the major facilitators of the mammalian immune response. Therefore, a reduction in the activation of T lymphocytes by the molecules of the invention or a change in the polarisation of such T lymphocytes, as described above, will result in an overall modulation of the immune response and in particular in changes in the levels of cytokines associated with the-immune response. For example, the molecules in the invention may have a generally immunosuppressant effect.

It will be apparent to a person skilled in the art that a modulation in the immune response, such as an immunosuppressant effect, can be measured using any one of a variety of methods. A decrease in, or modulation of, the overall immune response can be measured by looking for a reduction in the levels of pro-inflammatory cytokines produced most rapidly in response to TLR stimulation, e.g. interleukin-1 and tumour necrosis factor α (TNFα), interferon-α, interferon β, or cytokines such as IL-6 or IL-12 typically produced at intermediate times after infection. The molecules of the invention may also lead to an increase in the level of anti-inflammatory cytokines e.g. IL-10 or TGF-β.

In one embodiment, the molecules of the invention will reduce the levels of pro-inflammatory cytokines or increase the levels of anti-inflammatory cytokines by at least about 20% compared to a standard assay in the absence of a DC differentiation and maturation inhibiting molecule. In further embodiments, the inhibition of DC differentiation and maturation may reduce the levels of pro-inflammatory cytokines or increase the levels of anti-inflammatory cytokines by at least about 30%, 40%, 50%, 60%, 70%, 80%, 90% or more.

An immunosuppressant effect may also be assessed by a variety of other methods, for example a localised reduction in inflammation or a reduction in the size or activity of generated antigen-specific T and B cell pools.

Arthropods Front Which the Molecules of the Invention May Be Isolated

The molecules of the invention may be isolated from an arthropod. An “arthropod” is defined as an animal belonging to the phylum Arthropoda, and includes insects, crustaceans and arachnids. Arthropods are characterised by a segmented body and a hard exoskeleton made of chitin.

Within one aspect of the invention, the molecule of the invention may be isolated from a haematophagous arthropod. The term “haematophagous arthropod” includes all arthropods that take a blood meal from a suitable host. This includes insects, ticks, lice fleas and mites. They are commonly known as blood feeding arthropods, and these two terms will be used interchangeably throughout this application.

Within a further aspect of the invention, the isolated haematophagous arthropod may be a tick. The term “tick” is the common name given to small arachnids in the superfamily Ixodoidea, which is included within the haematophagous arthropods. Ticks are ectoparasites, and live on the blood of mammals, birds, and reptiles.

There are approximately 900 species of tick, which are found throughout the world. Different tick species are characterised by their preferential habitat and by their geographical distribution. Most tick species can feed on a variety of host species, including humans. As discussed above, arthropods, and particularly ticks may inhibit the host's immune system by inoculating the host with a variety of anti-inflammatory and immunomodulatory components.

This isolated DC modulatory molecule of the present invention may be isolated from any known tick species, including species within the groups Ixodinae, Bothriocrotoninae, Amblyomminae, Haemaphysalinae, Rhipicephalinae, Hyalomminae, Nuttalliellidae, Argasinae, Otobinae, Antricolinae, Nothoaspinae and Ornithodorinae, for example, any one of the following tick species: Rhipicephalus appendiculatus, Rhipicephalus sanguineus, Rhipicephalus bursa, Amblyomma americanum, Amblyomma cajennense, Amblyomma hebraeum, Amblyomma variegatum, Rhipicephalus (Boophilus) microplus, Rhipicephalus (Boophilus) annulatus, Rhipicephalus (Boophilus) decoloratus, Dermacentor reticulatus. Dermacentor andersoni, Dermacentor marginatus, Dermacentor variabilis, Haemaphysalis inermis, Haemaphysalis leachii, Haemaphysalis punctata, Hyalomma anatolicum anatolicum, Hyalomma dromedarii, Hyalomma marginatum marginatum, Ixodes ricinus, Ixodes persulcatus, Ixodes scapularis, Ixodes hexagonus, Argas persicus, Argas reflexus, Ornithodoros erraticus, Ornithodoros moubata moubata, Ornithodoros moubata porcinus, and Ornithodoros savignyi.

Protein of the Invention

The molecule of the invention may be a protein. As discussed in detail in Examples 3-18, the inventors have identified and isolated an arthropod protein which inhibits mammalian DC differentiation and maturation from tick species Rhipicephalus appendiculatus. This protein is referred to herein as Japanin, and its amino acid sequence is shown in FIG. 15 and SEQ ID NO: 2.

Therefore, in one aspect of the invention, the molecule of the invention may comprise:
i) a protein comprising the amino acid sequence of SEQ ID NO: 2;
ii) a homologue of a protein as defined in i);
iii) an active fragment of a protein as defined in i) above or of a homologue as defined in ii) above; or
iv) a functional equivalent of i), ii) or iii).

The molecule of the invention may be a protein consisting of the amino acid sequence of SEQ ID NO:2, or an active fragment thereof.

The term “functional equivalent” is used herein to describe any molecule possessing ability to modulate, and preferably inhibit, the differentiation and maturation of DCs in a manner corresponding to the full length Japanin protein comprising the amino acid sequence of SEQ ID NO:2, using the assays described above. This includes synthetically produced proteins, synthetic variants of the protein, protein molecules of a different sequence which confer corresponding activity, naturally occurring non-protein molecules with corresponding activity, and synthetic non-protein molecules with corresponding activity.

In particular, synthetic molecules that are designed to mimic the tertiary structure or active site(s) of the Japanin molecule are considered to be functional equivalents. In one embodiment, functional equivalents possess the ability to modulate, and preferably inhibit, the differentiation and maturation of DCs, resulting in a decrease in T lymphocyte activation or modulation of T lymphocyte polarisation, as described above. In a further embodiment, functional equivalents possess the ability to modulate, and preferably inhibit, the differentiation and maturation of DCs, resulting in an inhibition of the immune response, as described above.

Glycosylation

As shown in Example 26, Japanin appears to be glycosylated, possibly at two sites. Asparagine residues that are part of consensus sequences for asparagine-linked glycosylation are located at positions 59 and 155 of the amino acid sequence of Japanin (SEQ ID NO: 2). These equate to positions 35 and 131 in the mature protein which lacks the leader peptide sequence.

Accordingly, proteins of the invention may be glycosylated at one or more positions. In one embodiment, the protein may be glycosylated at one, two, three or more positions. The protein of the invention may be N-glycosylated, although the protein may also be O-glycosylated at one or more positions.

Proteins of the invention may be glycosylated naturally. This may particularly be the case if the protein is produced naturally and isolated, or if the protein is produced recombinantly in a host cell which mirrors the glycosylation pattern of the organism in which the protein is naturally produced. Alternatively, it may be necessary to artificially glycosylate the protein of the invention. This may particularly be the case if the protein is chemically synthesised, or if the protein is produced recombinantly in an organism which does not mirror the natural glycosylation pattern of the protein. Further, additional glycosylation may occur in order to alter or improve the properties of the protein.

Lipocalin Structure & Lipid Binding Properties

As shown in Example 11, the inventors have discovered that Japanin is a lipocalin. The lipocalins are a family of proteins which share a similar structural fold. The characteristic lipocalin fold is an eight-stranded anti-parallel beta-barrel, which forms an internal ligand binding site. The lipocalins, in general, contain at least 2, more often 4, cysteine residues at spaced locations. The cysteine residues form internal disulphide bonds, which may stabilise the lipocalin fold. In one aspect of the invention, the molecule may be a lipocalin. Accordingly, homologues, fragments and functional equivalents, as included within the scope of the invention, may comprise the motif Cys/Tyr X Leu Trp, commonly found in tick lipocalins.

Given its putative lipocalin structure, the Japanin protein may be associated with a lipid or lipid-like molecule. The term “lipid” is intended to encompass any hydrophobic or amphiphilic molecule which is soluble in organic solvents but insoluble in water. This includes fats, oils, triacylglycerols, glycolipids, phospholipids and steroids, fatty acyls, fatty acids, glycerolipids, glycerophospholipids, sphingolipids, saccharolipids, polyketides, sterol lipids and prenol lipids. The term “lipid-like molecule” encompasses any molecule having similar or identical properties to a lipid. Also included within the term “lipid-like molecule” is any lipid complexed to a non-lipid molecule. This includes glycolipids, phospolipids, phosphoglycolipids, labeled lipids and acetylated lipids.

In vivo, the protein of the invention may become bound to a lipid molecule during or immediately after its folding in the endoplasmatic reticulum or at a subsequent stage after its export from this location.

Within the scope of the invention, the protein of the invention may become associated with the lipid during its production. For example, if the protein of the invention is isolated from its natural source, or produced recombinantly, the protein may automatically become associated with the lipid without any intervention being required. Alternatively, the lipid may be added to a composition comprising the protein of the invention in order to allow complexation to occur between the lipid and the protein. In particular, if the protein has been chemically synthesised, the lipid may be added exogenously to a composition comprising the protein in order for complexation to occur.

As described above, the protein of the invention may be “associated” or “complexed” with the lipid. These terms are used interchangeably herein to relate to any sort of contact between the lipid and the protein of the invention. In particular, there may be an interaction between the protein of the invention and the lipid. In one embodiment, this interaction may be purely structural i.e. the lipid may fit into a binding pocket within the protein through a tessellating relationship. In another embodiment, the lipid may physically interact with the protein through any attractive force. Such attractive forces may include electrostatic interactions, hydrophobic interactions, hydrophilic interactions, van der Waals forces, hydrogen bonds, and covalent interactions. The interaction between the lipid and the protein may be formed from a combination of a structural interaction and an attractive force.

In one embodiment, the protein of the invention may be associated with a lipid. In another embodiment the lipid may be a steroid or a sterol, for example cholesterol. In another embodiment, the lipid may be a metabolite of cholesterol, such as vitamin D3. As shown in Example 23, Japanin has been shown to bind to cholesterol. In one embodiment, the protein of the invention may bind to a metabolite of cholesterol. In particular, the invention thus provides a complex which comprises or consists of Japanin and a lipid, for example cholesterol or a metabolite of cholesterol.

Functional equivalents of the Japanin protein, homologues and fragments may also associate with a lipid or other hydrophobic molecule(s) including a lipid-like molecule. In particular, functional equivalents of the Japanin protein, homologues and fragments may associate with cholesterol or with a metabolite of cholesterol.

As Japanin has been shown to bind to cholesterol, the inventors conceive that molecules which have been engineered to carry a lipid, but do not retain the biological activity of Japanin may have useful properties. The invention therefore includes a carrier molecule which binds a lipid and targets a receptor on the surface of DCs. Such a carrier molecule does not itself possess biological activity. In one embodiment, a carrier molecule may be produced by engineering Japanin to prevent its biological activity. In another embodiment, the lipid carried by the carrier molecule may confer a biological function by binding to a cellular receptor. The term “functional equivalent” thus includes carrier molecules.

Receptor Binding

As shown in Example 24, Japanin is thought to bind to a C-type lectin cell surface receptor. This suggests that Japanin functions to modulate, and preferably inhibit, the differentiation and maturation of dendritic cells by binding to a receptor on the surface of the target cell and triggering an internal cell signalling pathway which causes the inhibition.

In one embodiment, the protein may bind to a receptor on the outer surface of a target cell, for example a DC. In another embodiment, the protein may bind to a divalent cation-dependent receptor, in particular a C-type lectin receptor. In one embodiment, the protein may bind to a receptor and mimic the natural ligand for the receptor. It will be apparent to a person skilled in the art that any part of the protein may bind to the receptor. In particular, if the protein is glycosylated and/or bound to a lipid molecule, it may be the carbohydrate moiety or the associated lipid which binds to the receptor on the target cell.

In one embodiment there is included within the invention a complex comprising or consisting of a protein of the invention and the receptor. In another embodiment, the complex may comprise or consist of a protein of the invention, the receptor, for example a divalent cation-dependent receptor such as a C-type lectin receptor and a lipid, for example cholesterol or a metabolite of cholesterol.

Homologues and Fragments

As mentioned above, the invention includes homologues and active fragments of the Japanin protein which is shown as the amino acid sequence of SEQ ID NO: 2. The invention also includes functional equivalents of these homologues and fragments.

The term “homologue” is intended to include reference to paralogues and orthologues of the Japanin sequence that is disclosed in SEQ ID NO: 2 that retain the ability to modulate, and preferably inhibit, the differentiation and maturation of DCs. Homologues may possess the ability to modulate, and preferably inhibit, the differentiation and maturation of DCs, resulting in a decrease in T lymphocyte activation or modulation of T lymphocyte polarisation, as described above. In a further embodiment, homologues possess the ability to inhibit the differentiation and maturation of DCs, resulting in an inhibition of the immune response, as described above. In another embodiment, homologues may possess the ability to bind a lipid, for example cholesterol or a metabolite of cholesterol and/or the ability to bind a membrane-bound receptor, for example a divalent cation-dependent receptor such as a C-type lectin receptor.

Homologues may be derived from tick species other than Rhipicephalus appendiculatus, including Rhipicephalus sanguineus, Rhipicephalus bursa, Amblyomma americanum, Amblyomma cajennense, Amblyomma hebraeum, Ambylomma variegatum, Rhicephalus (Boophilus) microplus, Rhicephalus (Boophilus) annulatus, Rhicephalus (Boophilus) decoloratus, Dermacentor reticulatus, Dermacentor andersoni, Dermacentor marginatus, Dermacentor variabilis, Haemaphysalis inermis, Haemaphysalis leachii, Haemaphvsalis punctata. Hvalomma anatolicum anatolicum, Hyalomma dromedarii, Hyalomma marginatum marginatum, Ixodes ricinus, Ixodes persulcatus, Ixodes scapularis, Ixodes hexagonus, Argas persicus, Argas reflexus, Ornithodoros erraticus, Ornithodoros moubata moubata, Ornithodoros moubata porcinus, and Ornithodoros savignyi. Homologues may also be derived from mosquito species, including those of the Culex, Anopheles and Aedes genera, particularly Culex quinquefasciatus, Aedes aegypti and Anopheles gambiae; flea species, such as Ctenocephalides felis (the cat flea); horseflies; sandflies; blackflies; tsetse flies; lice; mites.

In general, homologues may be derived from any known tick species, for example those within the groups Ixodinae, Bothriocrotoninae, Amblyomminae, Haemaphysalinae, Rhipicephalinae (including Hyalomminae), Nuttalliellidae, Argasinae, Otobinae, Antricolinae, and Ornithodorinae.

Methods for the identification of homologues of the isolated Japanin protein sequence described herein will be clear to those of skill in the art. For example, homologues may be identified by homology searching of sequence databases, both public and private. Conveniently, publicly available databases may be used, although private or commercially-available databases will be equally useful, particularly if they contain data not represented in the public databases. Primary databases are the sites of primary nucleotide or amino acid sequence data deposit and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.ac.jp/), the SWISS-PROT protein database (http://expasy.hcuge.ch/), PIR (http://pir.georgetown.edu/), TrEMBL (http://www.ebi.ac. uk/), the TIGR databases (see http://www.tigr.org/tdb/index.html), the NRL-3D database (http://www.nbrfa.georgetown.edu), the Protein Data Base (http://www.rcsb.org/pdb), the NRDB database (ftp://ncbi.nlm.nih.gov/pub/nrdb/README), the OWL database (http://www.biochem.ucl.ac.uk/bsm/dbbrowser/OWL/) and the secondary databases PROSITE (http://expasy.hcuge.ch/sprot/prosite.html), PRINTS (http://iupab.leeds.ac. uk/bmb5dp/prints.html), Profiles (http://ulrec3.unil.ch/software/PFSCAN_form.html), Pfam (http://www.sanger.ac.uk/software/pfam), Identify (http://dna.stanford. edu/identify/) and Blocks (http://www.blocks.fhcrc.org) databases. Examples of commercially-available databases or private databases include PathoGenome (Genome Therapeutics Inc.) and PathoSeq (Incyte Pharmaceuticals Inc.).

Typically, greater than 30% identity between two polypeptides (preferably, over a specified region) is considered to be an indication of functional equivalence and thus an indication that two proteins are homologous. In one embodiment, proteins that are homologues have a degree of sequence identity with the Japanin protein sequence of SEQ ID NO: 2 of greater than 60%. In other embodiments, homologues have degrees of identity of greater than 70%, 80%, 90%, 95%, 98% or 99%, respectively with the isolated arthropod protein sequence of SEQ ID NO: 2. Percentage identity, as referred to herein, is as determined using BLAST version 2.1.3 using the default parameters specified by the NCBI (the National Center for. Biotechnology Information; http://www.ncbi.nlm.nih.gov/) [Blosum 62 matrix; gap open penalty=11 and gap extension penalty=1].

Homologues of the Japanin protein sequence of SEQ ID NO: 2 include mutants containing amino acid substitutions, insertions or deletions from the wild type sequence, provided that modulation, and preferably inhibition, of the differentiation and maturation of DCs demonstrated by the wild type protein sequence is retained. Mutants may possess the ability to modulate, and preferably inhibit, the differentiation and maturation of DCs, resulting in a decrease in T lymphocyte activation or modulation of T lymphocyte polarisation, as described above. In a further embodiment, mutants possess the ability to modulate, and preferably inhibit, the differentiation and maturation of DCs, resulting in an inhibition of the immune response, as described above.

Mutants thus include proteins containing conservative amino acid substitutions that do not affect the function or activity of the protein in an adverse manner. This term is also intended to include natural biological variants (e.g. allelic variants or geographical variations within the species from which the isolated arthropod proteins of the invention are derived). Mutants with improved activity in the modulation or inhibition of the differentiation and maturation of DCs compared to that of the wild type protein sequence may also be designed through the systematic or directed mutation of specific residues in the protein sequence.

As described in Example 20, the inventors have identified a Japanin homologue in Dermacentor andersoni. As described in Example 21, the inventors have identified homologues of Japanin from Rhipicephalus (Boophilus) microplus (2 homologues), Amblyomma americanum, and Rhipicephalus appendiculatus respectively. The sequences are shown in FIGS. 25-29, which correspond to SEQ ID NOs: 4, 6, 8, 10, and 12.

Accordingly, in a further aspect of the invention, the isolated molecule of the invention may comprise:
i) a protein comprising the amino acid sequence of any one of SEQ ID NOs: 4, 6, 8, 10 or 12,
ii) a homologue of a protein as defined in i);
iii) an active fragment of a protein as defined in i) above or of a homologue as defined in ii) above; or
iv) a functional equivalent of i), ii) or iii).

Although the inventors do not wish to be bound by theory, it is postulated that these sequences may not be full-length sequences. The invention thus provides that further amino acids may be present at the N-terminal and/or the C-terminal of the molecules comprising the amino acid sequences of SEQ ID NOS:4, 6, 8, 10 or 12.

The present invention also provides “active fragments” of the isolated Japanin molecule and of homologues of the isolated Japanin molecule. Included within this definition are any fragments which retain the ability to modulate or inhibit mammalian DC differentiation and modulation of the full-length Japanin molecule.

Included as such fragments are not only fragments of the isolated arthropod proteins that are defined herein as SEQ ID NOs: 2, 4, 6, 8, 10, and 12, but also fragments of homologues of this protein, as described above. Such fragments of homologues will typically possess greater than 60% identity with fragments of the isolated arthropod proteins of SEQ ID NOs: 2, 4, 6, 8, 10, and 12, although more preferred fragments of homologues will display degrees of identity of greater than 70%, 80%, 90%, 95%, 98% or 99%, respectively with fragments of the isolated arthropod proteins of SEQ ID NOs: 2, 4, 6, 8, 10, and 12.

These active fragments of the isolated arthropod proteins of SEQ ID NOs: 2, 4, 6, 8, 10, and 12, and fragments of homologues thereof modulate, and preferably inhibit, the differentiation and maturation of DCs. In one embodiment, fragments of the isolated arthropod proteins of SEQ ID NOs: 2, 4, 6, 8, 10, and 12 and fragments of homologues thereof modulate, and preferably inhibit, the differentiation and maturation of DCs resulting in a decrease in T lymphocyte activation or modulation of T lymphocyte polarisation, as described above. In a further embodiment, fragments of the isolated arthropod proteins of SEQ ID NOs: 2, 4, 6, 8, 10, and 12 and fragments of homologues thereof modulate, and preferably inhibit, the differentiation and maturation of DCs, resulting in an inhibition of the immune response, as described above. Fragments with improved activity in modulating or inhibiting the differentiation and maturation of DCs may, of course, be rationally designed by the systematic mutation or fragmentation of the wild type sequence followed by appropriate activity assays.

In one embodiment, homologues may possess the ability to bind a lipid, for example cholesterol or a metabolite of cholesterol and/or the ability to bind a membrane receptor, for example a divalent cation-dependent receptor such as a C-type lectin receptor.

In one embodiment, fragments of isolated arthropod proteins as described above may be at least about 100 amino acids in length. In further embodiments, fragments of isolated arthropod proteins as described above may be at least about 90, at least about 80, at least about 70, at least about 60, at least about 50, at least about 40, at least about 30, at least about 20, at least about 10 or at least about 5 amino acids in length.

Antibodies

The invention also provides an antibody which binds to a molecule of the invention and in particular to the Japanin protein, homologues, fragments and functional equivalent thereof described above. The antibody may be used as a reagent for the detection of the molecule. It may also be an antibody that neutralises the activity of the molecule in modulating or inhibiting the DC differentiation and maturation and is thus useful for therapeutic purposes, as described below. Included within this aspect of the invention are antibodies which bind to any of the functional equivalents, homologues and protein fragments included within the scope of the invention, as described above.

The invention also includes antibodies which bind to a carbohydrate moiety of the protein of the invention. In particular, the invention includes antibodies which bind to one or more of the carbohydrate moieties naturally attached to Japanin.

“Anticalins” are also included within the scope of the invention. These are molecules which are engineered from lipocalins to recognise and bind specific protein epitopes. In certain embodiments anticalins may take the form of peptides, glycopeptides or glycolipids. Herein, anticalins are included within the scope of the term “antibodies”. Anticalins are non-immunoglobulin-derived molecules which nevertheless recognise protein epitopes in a manner similar to antibodies.

If polyclonal antibodies are desired, a selected mammal, such as a mouse, rabbit, goat or horse, may be immunised with a molecule of the invention such as the Japanin protein, fragments, homologues or functional equivalents thereof. If desired, the molecule can be conjugated to a carrier protein. Commonly used carriers include bovine serum albumin, thyroglobulin and keyhole limpet haemocyanin. The coupled molecule is then used to immunise the animal. Serum from the immunised animal is collected and treated according to known procedures, for example by immunoaffinity chromatography.

Monoclonal antibodies to the molecules of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

As used herein, the term “antibody” includes fragments of antibodies, such as Fab, F(ab′)₂ and Fv fragments, which also bind specifically to a DC modulatory molecule. The term “antibody” further includes chimeric and humanised antibody molecules having specificity for the molecules of the invention and in particular for the Japanin protein, homologues, and fragments thereof. Chimeric antibodies are antibodies in which non-human variable regions are joined or fused to human constant regions (see, for example, Liu et al., Proc. Natl. Acad. Sci. USA, 84, 3439 (1987)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)).

In some cases, it may be desirable to attach a label group to the antibody, e.g. to facilitate detection. The label may be an enzyme, a radiolabel, a compound such as biotin, or a fluorochrome.

Fusion Proteins

The invention also includes a fusion protein comprising a molecule of the invention, in particular the Japanin protein, homologues, fragments or functional equivalents thereof, that is genetically fused or chemically linked to one or more peptides, polypeptides or other molecules. The purpose of the additional peptide or polypeptide or molecule may be to aid detection, expression, separation or purification of the protein or it may lend the protein additional properties as desired. Examples of potential fusion partners include beta-galactosidase, glutathione-S-transferase, luciferase, a polyhistidine tag, a T7 polymerase fragment and a secretion signal peptide. The fusion partner may also extend the life of the molecules in vivo, e.g. an Fc fragment. Examples of fusion proteins are provided in Examples 15-18.

Other potential fusion partners include potential biopharmaceuticals, such as proteins or other molecules that are being developed for use as drugs to treat specific diseases. Further potential fusion partners include antigens that will target the molecule of the invention to cells within the immune system, such as DCs. For example, fusion partners may include a self or foreign antigen or an allergen which may be fused to the molecule to deliver it to the DCs in vivo. Further fusion partners may include molecules that bind to a different cell surface component of the DC to facility delivery to the DC. In some cases, multiple fusion partners may be included. Examples of such antigens are discussed in more detail below.

Nucleic Acids

The invention also includes a nucleic acid molecule comprising a nucleic acid sequence encoding a molecule of the invention. Included within the term “nucleic acid molecule” is intended to be DNA molecules, RNA molecules and mixed DNA-RNA molecules. Further included within this definition are genomic DNA, cDNA molecules, mRNA molecules and RNA and DNA molecules containing modified bases. As will be apparent to a person skilled in the art, the degeneracy of the genetic code provides that there will be a number of different nucleic acid sequences which are capable of encoding the defined protein sequence of an isolated protein, protein fragment or functional equivalent thereof, as included within the scope of the invention. The invention also includes a nucleic acid molecule encoding a fusion protein, such as the fusion proteins described above.

In one aspect of the invention, the nucleic acid molecule comprising a nucleic acid sequence encoding a molecule of the invention may comprise or consist of SEQ ID NO: 1, or a degenerate sequence thereof. In further aspects of the invention, the nucleic acid molecule may comprise any one of SEQ ID NOs: 3, 5, 7, 9 or 11 or a degenerate sequence thereof. An example of a degenerate sequence is the nucleic acid molecule of SEQ ID NO: 32 which encodes the same Dermacentor andersonii protein of SEQ ID NO:4 as the nucleic acid molecule of SEQ ID NO3:

The invention also provides an antisense nucleic acid molecule which hybridises under high stringency hybridisation conditions to a nucleic acid molecule comprising a nucleic acid sequence encoding a molecule of the invention, in particular an isolated arthropod protein, homologue, fragment or a functional equivalent thereof, as described above. High stringency hybridisation conditions include overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Antisense nucleic acid molecules include antisense DNA oligonucleotides, and RNA oligonucleotides including siRNA.

The invention also includes a vector containing a nucleic acid molecule comprising a nucleic acid sequence encoding an isolated arthropod protein, homologue, fragment or a functional equivalent thereof or an antisense nucleic acid molecule which hybridises under high stringency hybridisation conditions to said nucleic acid molecule. Said vectors include cloning and expression vectors. Such expression vectors may incorporate the appropriate transcriptional and translational control sequences, for example enhancer elements, promoter-operator regions, termination stop sequences, mRNA stability sequences, start and stop codons or ribosomal binding sites, linked in frame with the nucleic acid molecules of the invention. These control sequences are provided by way of example only, and are not intended to be limited.

Additionally, it may be convenient for a recombinant protein to be secreted from certain hosts. Accordingly, further components of such vectors may include nucleic acid sequences encoding secretion, signalling and processing sequences.

Vectors according to the invention may include plasmids and viruses (including both bacteriophage and eukaryotic viruses), as well as other linear or circular DNA carriers, such as those employing transposable elements or homologous recombination technology. Particularly suitable viral vectors include baculovirus-, lentivirus-, adenovirus- and vaccinia virus-based vectors.

The invention also includes a host cell containing a vector, a nucleic acid molecule or an antisense nucleic acid encoding a molecule of the invention, in particular an arthropod protein, homologue, fragment or functional equivalent which modulates, and preferably inhibits, differentiation and maturation of DCs. Within the scope of the invention, any type of host cell may be utilised. In one embodiment, the host cell may be a prokaryotic host cell. Within this embodiment, the prokaryotic host cell may be an E. coli host cell. In another embodiment, the host cell may be a eukaryotic host cell. Within this embodiment the host cell may be a eukaryotic yeast cell. In a further embodiment the host cell may be a mammalian host cell. In a still further embodiment the host cell may be an insect cell, and within this embodiment the expression system may be the baculovirus expression system.

A variety of techniques may be used to introduce the vectors or nucleic acids of the present invention into host cells. Suitable transformation or transfection techniques are well described in the literature (Sambrook et al, 1989; Ausubel et al, 1991; Spector, Goldman & Leinwald, 1998). In eukaryotic cells, expression systems may either be transient (e.g. episomal) or permanent (chromosomal integration) according to the needs of the system.

In a further embodiment of the invention, there is provided a method of preparing a molecule of the invention, in particular an isolated arthropod protein, homologue, fragment or functional equivalent which modulates; and preferably inhibits, DC differentiation and maturation comprising:
i) culturing a host cell containing a vector comprising a nucleic acid sequence which encodes a molecule of the invention, in particular an arthropod protein, homologue, fragment or functional equivalent, which modulates or inhibits DC differentiation and maturation, according to the invention, under conditions whereby said protein is expressed; and
ii) recovering said protein thus produced.

Within this aspect of the invention, the conditions required for protein expression will vary depending upon the host cell system, the vector and the subsequent method of protein recovery. The examples disclose a particular process for production and recovery of the isolated proteins of the invention. Variation in such conditions will be apparent to a person skilled in the art.

Pharmaceutical Compositions

Due to the identified activity of the molecules of the present invention in the modulation, and preferably inhibition, of the differentiation and maturation of DCs, the molecules, proteins, nucleic acids, antisense nucleic acids, vectors, host cells and antibodies of the present invention are intended to be used as therapeutics.

The invention provides a pharmaceutical composition comprising an isolated DC modulatory molecule such as an arthropod protein which modulates, and preferably inhibits, the differentiation and maturation of DCs, or a functional equivalent thereof, a nucleic acid encoding such a molecule, the vector containing said nucleic acid, the host cell containing said vector or an antibody which binds to said molecule and a pharmaceutically acceptable carrier.

The term “pharmaceutically acceptable carrier”, as used herein, includes genes, polypeptides, antibodies, liposomes, polysaccharides, polylactic acids, polyglycolic acids and inactive virus particles or indeed any other agent provided that the excipient does not itself induce toxicity effects or cause the production of antibodies that are harmful to the individual receiving the pharmaceutical composition. Pharmaceutically acceptable carriers may additionally contain liquids such as water, saline, glycerol, ethanol or auxiliary substances such as wetting or emulsifying agents, pH buffering substances and the like. Excipients may enable the pharmaceutical compositions to be formulated into tablets, pills, dragees, capsules, liquids, gels, syrups, slurries, suspensions to aid intake by the patient. A thorough discussion of pharmaceutically acceptable carriers is available in Remington's Pharmaceutical Sciences (Mack Pub. Co., N.J. 1991).

In one embodiment, the pharmaceutical composition may include one or more lipid molecules which interact with the protein. In one specific embodiment, this lipid molecule may be cholesterol or a metabolite of cholesterol, such as vitamin D3. In another embodiment the pharmaceutical composition may include a complex which comprises or consists of Japanin and a lipid, for example cholesterol or a metabolite of cholesterol. In a further embodiment, the pharmaceutical composition may include a complex which comprises or consists of Japanin, a lipid, for example cholesterol or a metabolite of cholesterol, and a receptor, for example a divalent cation-dependent receptor such as a C-type lectin receptor.

In one aspect of the invention, the pharmaceutical composition may also include one or more additional therapeutic agents. Included within this aspect of the invention are any additional therapeutic agents which the skilled person might consider would be advantageous for co-administration with the molecules of the invention. In particular, said additional therapeutic agent may comprise an anti-inflammatory agent, an immunomodulatory agent, an immunosuppressant, a cytokine, a cytokine mimetic or a cytokine binding protein. In particular embodiments, the one or more additional therapeutic agents may include an anti-inflammatory agent.

Methods of Treatment

The present invention provides an isolated molecule, such as a protein, which modulates, and preferably inhibits, the differentiation and maturation of mammalian DCs, a nucleic acid encoding such a protein, an antisense nucleic acid, the vector containing said nucleic acid or antisense nucleic acid, the host cell containing said vector, an antibody which binds to said protein or molecule or a pharmaceutical composition comprising said molecule, protein, nucleic acid, vector, host cell or antibody for use in therapy.

As used herein, the term “therapy” includes use of the proteins, molecules, nucleic acids, vectors, host cells, antibodies or pharmaceutical compositions described herein for the benefit of a human or animal patient. Specifically this term includes therapeutic treatment, prophylactic treatment, diagnosis, and vaccination. This list is provided by way of illustration only, and is not intended to be limiting.

The molecules, proteins, nucleic acids, vectors, host cells, antibodies or pharmaceutical compositions of the present invention may be used for the treatment of any animal. In some embodiments, this animal may be a mammal. In further embodiments, this mammal may be a cow, pig, sheep, cat, dog or rabbit. In further embodiments, the mammal may be a human.

In another embodiment, there is provided the use of an isolated molecule which modulates, and preferably inhibits, the differentiation and maturation of mammalian DCs, such as an isolated arthropod protein which modulates, and preferably inhibits, the differentiation and maturation of mammalian DCs, a nucleic acid of encoding such a protein, an antisense nucleic acid binding to the nucleic acid encoding such a protein, the vector containing said nucleic acid or antisense nucleic acid, the host cell containing said vector, an antibody which binds to said protein or molecule or a pharmaceutical composition comprising said protein, molecule, nucleic acid, vector, host cell or antibody in the manufacture of a medicament for treating diseases associated with DC activity.

The invention also provides a method of treating an animal suffering from a disease associated with DC comprising administering to said animal a molecule which modulates, and preferably inhibits, the differentiation and maturation of DCs, such as an isolated arthropod protein which modulates, and preferably inhibits, the differentiation and maturation of mammalian DCs, a nucleic acid encoding such a protein, an antisense nucleic acid binding to the nucleic acid encoding such a protein, the vector containing said nucleic acid or antisense nucleic acid, the host cell containing said vector, an antibody which binds to said protein or molecule or a pharmaceutical composition comprising said protein, molecule, nucleic acid, vector, host cell or antibody.

Within the scope of the invention, the molecules, proteins, nucleic acids, vectors, host cells, antibodies or pharmaceutical compositions of the present invention may be administered to a patient using any one or more of a number of modes of administration. Such modes of administration are well known in the art and may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. Nanopatches may be used for transdermal administration of the molecules, proteins, nucleic acids, vectors, host cells, antibodies or pharmaceutical compositions of the present invention. Gene guns may also be used to administer the nucleic acids, vectors, or pharmaceutical compositions of the invention. The precise mode of administration will depend on the disease or condition to be treated.

In one embodiment, the molecules, proteins nucleic acids, vectors, host cells, antibodies or pharmaceutical compositions of the invention may be used in the treatment or prevention of autoimmune disorders, allergies or other hypersensitivity disorders, transplant rejection and graft-versus-host disease, and acute and chronic inflammatory diseases.

The autoimmune disorders include but are not limited to achlorhydra autoimmune chronic active hepatitis, Addison's disease, alopecia areata, amyotrophic lateral sclerosis (ALS, Lou Gehrig's Disease), ankylosing spondylitis, anti-GBM nephritis or anti-TBM nephritis, antiphospholipid syndrome, aplastic anemia, arthritis, asthma, atopic allergy, atopic dermatitis, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Balo disease, Behcet's disease, Berger's disease (IgA Nephropathy), bullous pemphigoid, cardiomyopathy, celiac disease, celiac sprue dermatitis, chronic fatigue immune deficiency syndrome (CFIDS), chronic fatigue immune dysfunction syndrome (CFIDS), chronic inflammatory demyelinating polyneuropathy, Churg Strauss syndrome, cicatricial pemphigoid, Cogan's syndrome, cold agglutunin disease, colitis, cranial arteritis, CREST syndrome, Crohn's disease, Cushing's syndrome, Dego's disease, dermatitis, dermatomyositis, dermatomyositis—juvenile, Devic's disease, type 1 diabetes, discoid lupus, Dowling-Dego's disease, Dressler's syndrome, eosinophilic fasciitis, epidermolysis bullosa acquisita, essential mixed cryoglobulinemia, Evan's syndrome, fibromyalgia, fibromyositis, fibrosing alveolitis, gastritis, giant cell artertis, glomerulonephritis, Goodpasture's disease, Grave's disease, Guillian-Barre syndrome, Hashimoto's thyroiditis, hemolytic anemia, Henoch-Schonlein purpura, hepatitis, Hughes syndrome, idiopathic adrenal atrophy, idiopathic pulmonary fibrosis, idiopathic thrombocytopenia purpura (ITP), IgA nephropathy, inflammatory demylinating polyneuropathy, insulin dependent diabetes (Type I), irritable bowel syndrome, juvenile arthritis, Kawasaki's disease, lichen planus, Lou Gehrig's disease, lupoid hepatitis, Lyme disease, Meniere's disease, mixed connective tissue disease, multiple myeloma, multiple sclerosis, myasthenia gravis, myositis, ocular cicatricial pemphigoid, osteoporosis, pars planitis, pemphigus vulgaris, pernicious anemia, polyarteritis nodosa, polychondritis, polyglandular syndromes, polyglandular autoimmune syndromes, polymyalgia rheumatica (PMR), polymyositis, polymyositis and dermatomyositis, primary agammaglobulinemia, primary biliary cirrhois, primary biliary cirrhosis, primary sclerosing cholangitis, psoriasis, Raynaud's phenomenon, Reiter's syndrome, rheumatoid arthritis, sarcoidosis, scleritis, scleroderma, Sjogren's syndrome, sticky blood syndrome, stiff-man syndrome, Still's disease, Sydenham's chorea, systemic lupus erythmatosis (SLE), Takayasu's arteritis, temporal arteritis/giant cell arteritis, ulcerative colitis, uveitis, vasculitis, vitiligo, Wegener's granulomatosis, and Wilson's syndrome.

The allergy or hypersensitivity disorder may be any known allergy or hypersensitivity disorder including type I, type II, type III, or type IV according to the Gell-Coombs classification, and the less commonly defined type V hypersensitivity disorders. Such disorders include but are not limited to atopy, asthma, ertyhroblastosis fetalis, Goodpasture's syndrome, autoimmune hemolytic anemia, serum sickness, Arthus reaction, systemic lupus erythematosus, contact dermatitis, tuberculin skin test, chronic transplant rejection, Graves disease, myasthenia gravis, systemic anaphylaxis, local anaphylaxis, allergic rhinitis, conjunctivitis, gastroenteritis, eczema, blood transfusion reactions, haemolytic disease of the newborn, rheumatoid arthritis, glomerulonephritis, contact dermatitis, atopic dermatitis, tubercular lesions, drug-induced hemolytic anemia, lupus nephritis, aspergillosis, polyarteritis, polymyositis, scleroderma, hypersensitivity pneumonitis, Wegener's granulomastosis, type I diabetes mellitus, urticaria/angioedema, or inflammation of the thyroid. The allergy or hypersensitivity disorder may be associated with infectious diseases including but not limited to tuberculosis, leprosy, blastomycosis, histoplasmosis, toxoplasmosis, leishmaniasis or other infections. Allergies that may be treated include but are not limited to allergic reactions to pollens (e.g. birch tree, ragweed, oil seed rape), food (e.g. nuts, eggs or seafood), drugs (e.g. penicillin or salicylates), insect products (e.g. bee or wasp venom or house dust mites) or animal hair, and man-made products such as latex. Other inflammatory diseases that may be treated include atherosclerosis or other cardiovascular disease, Alzheimer's disease, vasculisitis, myositis, encephalitis, reperfusion injury, type 2 diabetes, fatty liver disease, and wound healing, including the inflammatory phase, the process of angiogenesis, fibroplasmia and epithelialisation, and the remodeling phase.

In one embodiment, the molecules, proteins, nucleic acids, vectors, host cells, antibodies or pharmaceutical compositions of the invention may be used in the treatment or prevention of harmful conditions resulting from bodily fluids or tissues coming into contact with artificial or non-mammalian materials during the course of therapeutic or diagnostic procedures. Such procedures may be temporary or permanent and include but not be limited to the use of extracorporeal circuits including renal or hepatic haemodialysis, peritoneal dialysis, cardiopulmonary bypass and haemofiltration, indwelling catheters whether placed in blood vessels, the urinary bladder, the intrathecal space or any other hollow viscus, implanted prostheses including artificial joints, heart valves, endovascular stents, CSF shunts, vascular prostheses and coronary angioplasty catheters.

The molecules, proteins, nucleic acids, vectors, host cells, antibodies or pharmaceutical compositions of the invention are likely to have an immunosuppressant effect that may be useful in preventing transplantation rejection. The transplants may be isografts between the same individual, allografts between different members of the same species or xenografts between different species. The molecules of the invention may be useful in preventing rejection of a range of transplants including, but not limited to heart, lung, heart and lung, kidney, liver, pancreas, intestine, hand, cornea, skin graft including face replant and face transplants, islets of Langerhans, bone marrow transplants, blood transfusion, blood vessels, heart valves, bone and skin. The molecules, proteins, nucleic acids, vectors, host cells, antibodies or pharmaceutical compositions may be used to prevent graft-versus host disease following bone marrow transplantation.

Although the inventors do not wish to be bound by theory, it is postulated that the Japanin protein may be involved in inhibiting signalling pathways involved in cancer. In a further embodiment of the present invention, the molecules, proteins, nucleic acids, vectors, host cells, antibodies or pharmaceutical compositions of the invention may be used in the treatment of cancer. The invention also provides a method of treating an animal suffering from cancer comprising administering to said animal a molecule, protein, nucleic acid, vector, antibody, or pharmaceutical composition of the invention, as described above. Such treatment may involve the repolarisation or modulation of the immune response in cancer.

In particular, the cancer may be a haematological cancer such as lymphoma or leukaemia or multiple myeloma. Leukemias that may be treated according to the invention include acute lymphoblastic leukaemia (ALL), acute myelogenous leukaemia (AML), chronic myelogenous leukaemia (CML), chronic lymphocytic leukaemia (CLL) and hairy cell leukaemia. Lymphomas that may be treated according to the invention include Hodgkin's disease and non-Hodgkin's lymphoma. Related disorders may also be treated including myelodysplastic syndrome (MDS) which can culminate in ALL, myeloproliferative disease including polycythemia vera, essential thrombocytosis or myelofibrosis, and amyloid due to light-chain disease.

In further embodiments, the cancer may be a carcinoma, a sarcoma, or a blastoma. The invention contemplates the treatment of cancers of any organ including but not limited to cancers of the breast, lung, ovaries, pancreas, testes, skin, colon, brain, liver or cervix, as well as melanoma. A cancer that may be treated or prevented is histiocytoma and in particular canine cutaneous histiocytoma.

In a further embodiment of the present invention, the molecules, proteins, nucleic acids, vectors, host cells, antibodies or pharmaceutical compositions of the invention may be used in the treatment of infectious disease. In particular, such treatment may involve the repolarisation or modulation of the immune response to infections by pathogens such as viruses, bacteria and protozoa, e.g. the causative agents of HIV, TB and malaria, as well as other parasites.

Haematophagous arthropods, such as ticks are sources of infectious disease agents such as tick-borne encephalitis virus, Crimean-Congo haemorrhagic fever virus, Nairobi sheep virus, Borrelia burgdorferi (the agent of Lyme's disease), and Theileria parva (the agent of East Coast fever). It is postulated that Japanin may act to promote transmission of tick-borne diseases. The Japanin protein, homologues, fragments and functional equivalents thereof may therefore be useful in the vaccination of animals to induce an immune response to treat or prevent tick-borne diseases. In a further embodiment of the invention there is therefore provided a method of preventing transmission of an arthropod-borne infectious disease or treating an arthropod-borne infectious disease comprising administering to an animal a molecule of the invention, such as a protein, or a nucleic acid molecule, vector, host cell, antibody or pharmaceutical composition of the invention. The invention also provides a molecule of the invention, such as a protein, or a nucleic acid molecule, vector, host cell, antibody or pharmaceutical composition of the invention for use in preventing transmission of an arthropod-borne infectious disease or treating an arthropod-borne infectious disease. The arthropod may be a haematophagous arthropod. The arthropod-borne disease may be Lyme's disease, tick-borne encephalitis, Crimean-Congo haemorrhagic fever, Nairobi sheep virus or East coast fever.

The Japanin protein, homologues, fragments and functional equivalents thereof may also be useful as vaccines against the haematophagous arthropods themselves, as well as the diseases carried by them. The invention therefore further provides a method of vaccinating an animal against a haematophagous arthropod which may be a tick, comprising administering a molecule of the invention, such as a protein, or a nucleic acid molecule, vector, host cell, antibody or pharmaceutical composition to an animal. The invention also provides a molecule of the invention, such as a protein, or a nucleic acid molecule, vector, host cell, antibody or pharmaceutical composition of the invention for use in vaccinating an animal against a haematophagous arthropod which may be a tick.

As discussed above, it may be advantageous to administer molecules, proteins, nucleic acids, vectors, host cells, antibodies or pharmaceutical compositions of the invention in combination with one or more additional therapeutic agents such as an anti-inflammatory agent, an immunomodulatory agent, an immunosuppressant, a cytokine, a cytokine mimetic or a cytokine binding protein, or another biopharmaceutical developed for the treatment of any of the disorders mentioned above.

It may also be advantageous to administer the molecules, proteins, nucleic acids, vectors, host cells or antibodies of the invention with an antigen that will target them to DCs in vivo to modulate or inhibit the differentiation and maturation of DCs associated with the unwanted immune response. In this embodiment, the molecules, proteins, nucleic acids, vectors, host cells, antibodies or pharmaceutical compositions of the present invention may be administered to the patient in combination with a disease-associated element to aid targeting of the proteins, molecules, nucleic acids, vectors, host cells, antibodies or pharmaceutical compositions of the present invention to the appropriate DCs. In embodiments where the molecules, proteins, nucleic acids, vectors, host cells or antibodies of the invention already target DCs by binding to them specifically, a disease associated element may not be necessary.

The term “disease-associated element” is intended to encompass any component which is associated with the disease in a patient. The disease may include autoimmune disorders, allergies and other hypersensitivity reactions, transplant rejection and graft-versus-host disease, infectious diseases including those transmitted by ticks, cancers including haematological malignancies, and acute and chronic inflammatory diseases, as described above. The “disease associated element” may thus include: i) components associated with infectious agents, such as viruses, microbes, parasites and microbial toxins; ii) allergens that are non-self molecules associated with allergy; iii) non-self components associated with hypersensitivity reactions other than allergy; iv) self-components associated with autoimmune diseases; v) transplantation antigens from genetically-different members of the same species (alloantigens) or from different species (xenoantigens); and vi) tumour-associated antigens and tumour-specific antigens.

The term “disease associated element” also encompasses fragments and derivatives of these disease-associated elements. Such derivatives may includes detoxified agents, synthetic mimotopes and antigen comprising substitutions, additions or deletions in their structure, which are still capable of acting to direct the proteins, molecules, nucleic acids, vectors, host cells, antibodies or pharmaceutical compositions of the invention to the appropriate DCs.

Providing the animal with a disease-associated element will improve the specificity of the modulation or inhibition of DC differentiation and maturation associated with the disease. Targeting specific DCs in this manner is advantageous as it avoids the need to inhibit the overall immune response, and may therefore result in a reduced profile of side effects.

In one aspect of the invention, the molecules, proteins, nucleic acids, vectors, host cells, antibodies or pharmaceutical compositions of the invention may be administered separately from the disease associated element. Within this aspect, the molecules, proteins, nucleic acids, vectors, host cells, antibodies or pharmaceutical compositions of the invention and the disease associated element may be administered sequentially. In a another embodiment, the molecules, proteins, nucleic acids, vectors, host cells, antibodies or pharmaceutical compositions of the invention may be administered before the disease associated element. In a further embodiment, the molecules, proteins, nucleic acids, vectors, host cells, antibodies or pharmaceutical compositions of the invention may be administered after the disease associated element.

In a further embodiment, the molecules, proteins, nucleic acids, vectors, host cells, antibodies or pharmaceutical compositions of the invention and the disease associated element may be administered simultaneously. Within this aspect of the invention, the proteins, molecules, or antibodies of the invention may be bound to the disease associated element, for example in the form of a fusion protein.

The invention therefore also provides, a fusion protein comprising a molecule which modulates or inhibits DC differentiation and maturation according to the invention, and a disease associated element. A nucleic acid encoding such a fusion protein is also provided.

The invention also provides a pharmaceutical composition comprising a molecule, protein, nucleic acid, vector, host cell, antibody or pharmaceutical composition of the invention and a disease associated element and a pharmaceutically acceptable carrier.

The methods describe above involve the administration of the molecules of the invention to an animal in order to modulate, and preferably inhibit, the differentiation and maturation of DCs in the animal in vivo. The molecules may be administered alone or in combination with additional agents, including disease causing elements, that will target the molecule to DCs.

An alternative approach to the treatment of the diseases described above is to use the molecules of the invention for targeted therapy ex vivo. This approach involves delivering a molecule of the invention to DC in vitro to modulate the DCs and delivering the modulated DCs to the animal in need of treatment.

In a further aspect, the invention provides a method of modulating a DC, said method comprising contacting a DC with a molecule of the invention, such as the Japanin protein, homologues, fragments and functional equivalents thereof described above. A modulated DC produced using this method is also provided.

The invention also provides a method of treating or preventing a disorder associated with DC in an animal in need thereof wherein the method comprises administering the modulated DC produced by this method to an animal.

The molecule which modulates DC differentiation may be any molecule that modulates, and preferably inhibits, DC differentiation and maturation described above, including the Japanin protein, homologues, fragments and functional equivalents thereof. Nucleic acid molecules encoding these molecules may also be used, as may vectors comprising the nucleic acid molecules.

The DCs may be isolated directly from the animal in need or treatment. Alternatively, DC precursors, such monocytes of bone marrow progenitors may be isolated from the animal and used to generate DCs. Within this aspect of the invention, the DC or DC precursors may be autologous or allogeneic with respect to the animal into which the modulated DCs are to be introduced following treatment with a molecule of the invention.

The disorder associated with DC may be any of the diseases discussed above including autoimmune disorders, allergies and other hypersensitivity reactions, transplant rejection and graft-versus-host disease, infectious diseases including those transmitted by ticks, cancers including haematological malignancies, and acute and chronic inflammatory diseases It is contemplated that this method may be used to generate modulated DCs from a transplant donor to administer to the intended recipient of a transplant prior to transplantation with the aim or inducing unresponsiveness to the graft and thus reducing the need for immunosuppressants to be given.

Within this aspect of the invention, the DCs may also be contacted with a disease associated element, as described above, in order to target the molecule which modulates, and preferably inhibits, DC differentiation and maturation to the appropriate DCs. Alternatively, the modulated DCs may be administered to the animal in combination with a disease-associated element, such as those as described above.

Screening Methods

The identification of the cognate receptor of Japanin allows the receptor to be used in screening methods to identify potential agonists and antagonists of Japanin in order to identify any compounds which are potentially of therapeutic or other use.

Potential agonist or antagonist compounds may be isolated from, for example, cells, cell-free preparations, chemical libraries or natural product mixtures. For a suitable review of such screening techniques, see Coligan et al., Current Protocols in Immunology 1(2):Chapter 5 (1991).

Compounds that are most likely to be good antagonists are molecules that bind to Japanin's cognate receptor without inducing the biological effects induced by Japanin, and thus competitively inhibit the function of Japanin. As described above, the cognate receptor of Japanin is thought to be a divalent cation-dependent receptor which is a C-type lectin receptor which, upon binding of Japanin, induces an intracellular signalling pathway leading to inhibition of differentiation and maturation of dendritic cells. Potential antagonists include small organic molecules, peptides, polypeptides and antibodies that bind to the receptor without activating a signalling pathway, or by activating a negative signalling pathway. In particular, suitable potential antagonists include carbohydrate moieties and engineered Japanin molecules which have been engineered to reduce their function whilst retaining their binding affinity for the receptor. Further suitable compounds include antibodies to Japanin and antibodies to the carbohydrate moiety associated with Japanin, and anticalins which can be engineered for target specificity.

Compounds most likely to function as good agonists are compounds which bind to the cognate receptor of Japanin and induce the same intracellular signalling pathway as Japanin, thereby functioning to modulate, and preferably inhibit, the differentiation and maturation of dendritic cells in a similar way to Japanin. Examples of suitable potential agonists include Japanin molecules which have been engineered to increase their ability to activate the receptor and/or their binding affinity for the receptor.

The cognate receptor (a divalent cation-dependent receptor, possibly a C-type lectin) for use in such a screening technique may be free in solution, affixed to a solid support, borne on a cell surface or located intracellularly. In general, such screening procedures may involve using appropriate cells or cell membranes that express the receptor and that are contacted with a test compound to observe binding, or stimulation or inhibition of a functional response. The functional response of the cells contacted with the test compound is then compared with control cells that were not contacted with the test compound. Such an assay may assess whether the test compound results in the generation of a signal similar to that generated by activation of the receptor by Japanin, using an appropriate detection system. Since Japanin is believed to function by binding to a cell surface divalent cation-dependent receptor such as a C-type lectin receptor and inducing an intracellular signalling pathway, a screening method is likely to function most effectively if it involves the use of receptors on the cell surface, and the monitoring of the induction of an intracellular signal by the binding of the compound to the receptor.

In one embodiment, a method for identifying an agonist or antagonist compound of Japanin comprises:

(a) contacting a cell expressing the divalent cation-dependent receptor, for example a C-type lectin receptor, on its surface with a compound to be screened under conditions to permit binding to the receptor, wherein the receptor is capable of providing a detectable signal in response to the binding of a compound; and

(b) determining whether the compound binds to and activates or inhibits the receptor by measuring the level of a signal generated from the interaction of the compound with the receptor.

In certain embodiments, the compounds to be screened may be contacted with the receptor in the presence of a ligand. Such a ligand may be a lipid, for example cholesterol or a metabolite of cholesterol, such as vitamin D3.

In another embodiment, a method of identifying an antagonist compound of Japanin comprises:

(a) contacting a cell expressing the divalent cation-dependent receptor, for example a C-type lectin receptor, on its surface with Japanin, wherein the receptor is capable of providing a detectable signal in response to the binding of a Japanin, under conditions which allow Japanin to bind to its cognate receptor;

(b) measuring the level of a signal generated from the interaction of Japanin with the receptor;

(c) adding a compound to be screened under conditions to permit binding to the receptor; and

(d) determining the effect of the compound upon Japanin binding by measuring the change in the level of a signal generated from the interaction of the compound with the receptor.

In certain embodiments, any homologue or functional equivalent of Japanin, as discussed above, may be used in the screening methods described above.

In certain further embodiments, the compounds to be screened and/or Japanin may be contacted with the receptor in the presence of a ligand. Such a ligand may be a lipid, for example cholesterol or a metabolite of cholesterol.

The conditions indicated above may include the presence of culture medium, the presence of a solution containing a physiolocal concentration of Ca²⁺, and/or a pH of 7-8.

The detectable signal described above may include intracellular phosphorylation, nuclear localisation, gene expression and/or cytokine release.

In certain embodiments of the methods described above, simple binding assays may be used, in which the adherence of a test compound to a surface bearing a C-type lectin receptor is detected by means of a label directly or indirectly associated with the test compound or in an assay involving competition with a labelled competitor. In another embodiment, competitive drug screening assays may be used, in which neutralising antibodies that are capable of binding the divalent cation-dependent receptor, such as the C-type lectin receptor, specifically compete with a test compound for binding. In this manner, the antibodies can be used to detect the presence of any test compound that possesses specific binding affinity for the receptor.

A person skilled in the art will be able to devise assays for identifying compounds which act on the cognate receptor of Japanin. A technique which may be used to provide for high throughput screening of compounds having suitable binding affinity to the receptor (see International patent application WO84/03564). In this method, large numbers of different small test compounds are synthesised on a solid substrate, which may then be reacted with the receptor and washed. One way of immobilising the polypeptide is to use non-neutralising antibodies. Bound receptor may then be detected using methods that are well known in the art. Purified receptor molecules can also be coated directly onto plates for use in the aforementioned drug screening techniques.

The present invention also includes the agonists, antagonists and other compounds which are identified by the methods that are described above. These agonists, antagonists and other compounds may be form part of the pharmaceutical compositions described above, and may be used in the methods of treatment described above.

Various aspects and embodiments of the present invention will now be described in more detail by way of example. It will be appreciated that modification of detail may be made without departing from the scope of the invention.

### BRIEF DESCRIPTION OF FIGURES

FIG. 1 shows the effect of SGE from fed and unfed male and female Rhipicephalus appendiculatus ticks on the upregulation of CD86 expression by human DCs. F=female, M=male, 0/3/6=number of days feeding.

FIG. 2 shows the effect of SGE from female 3 day-fed Rhipicephalus appendiculatus ticks on the expression of maturation markers by CD1a⁺ cells. Black histograms show expression of maturation markers by CD1a⁺ cells treated with SGE and LPS, and grey histograms show expression of maturation markers by CD1a⁺ cells treated with LPS in the absence of SGE treatment.

FIG. 3 shows the effect of Q column-separated SGE on the expression of maturation markers by CD1a⁺ cells treated with LPS, and expression of maturation markers by CD1a⁺ cells in the absence of LPS treatment. QFT=material passing through a Q column at pH7. QFR=material retained on a Q column at pH7.

FIG. 4 shows the effect of SGE or QFT on CD86 upregulation in the presence of A) LPS and IFNγ, and B) poly(I:C) and TNFα.

FIG. 5 shows the effect of proteinase K treatment on the DC modulatory effect of QFT.

FIG. 6 shows the results of size fractionation on DC modulatory activity. Activity was assessed as the inhibition of CD86 upregulation in the presence of LPS.

FIG. 7 shows the results of polyacrylamide gel electrophoresis on the most active fragment obtained using size exclusion chromatography.

FIG. 8 shows the results of HPLC purification: A) elution profile from the HPLC column, and B) activity profile of the fractions eluted from the HLPC.

FIG. 9 shows the N-terminal 16 amino acids, sequenced by Edman degradation.

FIGS. 10 and 11 show the results of PCRs to clone the 3′ region of Japanin DNA.

FIG. 12 shows the consensus sequence for the 3′ region of Japanin.

FIG. 13a shows primer design for the amplification of the 5′ region of Japanin.

FIG. 13b shows the results of PCR amplification of the 5′ region of Japanin. Lane “PCR” is untreated PCR product. The two 500bp lanes labelled “cleaned PCR” are different volumes following column clean-up.

FIG. 14 shows the 5′ cDNA sequence of Japanin.

FIG. 15 shows the full length Japanin cDNA, and the 176 amino acid protein it encodes.

FIG. 16 shows potential cleavage sites located within the full length Japanin sequence.

FIG. 17 shows DC modulatory activity of insect cell supernatant, obtained from cells containing a Japanin expressing vector.

FIG. 18 shows the presence of activity in various fractions following ammonium sulphate precipitation of supernatant collected from Japanin containing insect cells.

FIG. 19 shows a silver stained SDS-PAGE gel showing the presence of Japanin.

FIG. 20 shows a western blot which confirms the presence of the His-tag used to isolate Japanin.

FIG. 21 shows the DC modulatory effect of Japanin-TEV-his supernatant.

FIG. 22 shows DC modulatory activity of purified His-tagged Japanin.

FIG. 23 shows the effect of Japanin on the expression of CD1a and CD14 by DCs.

FIG. 24 shows the reduction of T cell proliferation induced by Japanin in an MLR assay.

FIG. 25 shows a pairwise alignment of Japanin with a Japanin homologue identified in Dermacentor andersoni (D. andersoni E1244)

FIG. 26 shows a pairwise alignment of Japanin with a Japanin homologue identified in Rhipicephalus microplus (R. microplus RM-CK185494).

FIG. 27 shows a pairwise alignment of Japanin with a Japanin homologue identified in Amblyomma americanum (A. americanum CX766068).

FIG. 28 shows a pairwise alignment of Japanin with a Japanin homologue identified in Rhipicephalus appendiculatus (R. appendiculatus CD796501).

FIG. 29 shows a pairwise alignment of Japanin with a Japanin homologue identified in Rhipicephalus microplus (R. microplus CV443471).

FIG. 30 shows mass spectroscopy data obtained from GC-MS analysis.

FIG. 31 shows the binding of ³H-cholesterol to Japanin.

FIG. 32 shows FACS analysis of the binding of fluorescently labelled Japanin to day 5 monocyte-derived dendritic cells (FIG. 32a), monocytes (FIG. 32b), bone marrow derived dendritic cells (FIG. 32c), day 1 monocyte derived dendritic cells (FIG. 32d), monocyte-derived dendritic cells in the presence of mannan (FIG. 32e), and in the presence of EDTA (FIG. 32f).

FIG. 33 shows the N-glycosylation of Japanin.

FIG. 34 shows the effect of Japanin upon inhibition of monocyte-derived dendritic cell maturation in the presence of LPS (FIG. 34a), IFNγ (FIG. 34b), TNFα (FIG. 34c), soluble CD40L (FIG. 34d), IFNα (FIG. 34e), and CD097, a TLR7/8 ligand (FIG. 34f).

FIG. 35 shows the effect of Japanin upon dendritic cell secretion of TNFα.

### EXAMPLES

Materials and Methods

Ticks

Rhipicephalus appendiculatus ticks were reared at the Centre of Ecology and Hydrology, Oxford. Feeding was performed by placing them on the shaved backs of guinea pigs in gauze-covered retaining chambers.

Salivary Gland Extract Preparation

Following 1-6 days of feeding, ticks were carefully detached from the guinea pigs and their salivary glands were dissected out under a microscope. The glands were briefly rinsed in cold phosphate-buffered saline (PBS; Oxoid Ltd.), transferred to 1.5 ml microcentrifuge tubes and stored at −70° C. until required. Salivary gland extract (SGE) was prepared by homogenising the glands in PBS using a Dounce homogeniser. The homogenate was centrifuged at ≧10000 g for 3 minutes, and the supernatants collected and stored at −20° C. until required.

Cell Culture

Cell culture media and supplements were, unless otherwise stated, from PAA. This includes foetal calf serum (FCS), of which batch #A04304-0511 was employed throughout. Mammalian cell culture was at 37° C., 5% CO2. Insect cell culture was at 28° C. in Sf900 II media (Invitrogen). With the exception of co-transfection cultures, liquid culture was performed in conical flasks, with 100-130rpm orbital shaking.

Dendritic Cells

Human dendritic cells (DC) were generated from peripheral blood monocytes isolated from healthy adult donors. Briefly, Buffy coats (National blood service, Bristol) were mixed 1:2 (v/v) with Ca2+/Mg2+-free Hanks buffered salt solution (HBSS), carefully layered on to Lymphoprep (Axis Shield) and centrifuged at 800 g for 30 minutes (at 22° C.). The peripheral blood mononuclear cell (PBMC) layer formed at the interface between the HBSS/Buffy coat mixture and Lymphoprep was carefully collected and washed three times with HBSS to remove platelets. Monocytes were isolated from PBMC either by transient adherence or by negative selection with magnetic beads.

For isolation of monocytes by transient adherence, the PBMC pellets were resuspended in RPMI-5, consisting of RPMI 1640 supplemented with 5% human AB+serum (National blood service), 2 mM L-glutamine, 100 U/ml penicillin and 100 μg/ml streptomycin. PBMC were plated at 1×107 cells/ml, 10 ml/plate in 100 mm cell culture-treated Petri dishes (BD Biosciences) and incubated for 45 minutes at 37° C., 5% CO2. Non-adherent cells were then removed by washing the plates three times with HBSS, and 10 ml of RPMI-5 added back to the plates. After 1 day of culture, human GM-CSF (John Radcliffe Hospital pharmacy) was added to a concentration of 1000 U/ml and human IL-4 (Peprotech) was added to a concentration of 20 ng/ml. After 3 days of culture, and again after 5 days, one third of the total volume was replaced with freshly prepared GM-CSF+IL-4-supplemented RPMI-5.

For isolation of monocytes by negative selection (in other words, by the removal of non-monocytes), the PBMC pellets were resuspended in Ca2+/Mg2+-free HBSS, and monocytes were then isolated using the Dynal Monocyte Negative Isolation kit (Invitrogen), in accordance with the manufacturer's instructions. Purified monocytes were resuspended at 5×105/ml in DC-RPMI: RPMI 1640 supplemented with 10% FCS, 2 mM L-glutamine, 100 U/ml penicillin, 100 μg/ml streptomycin, 1000 U/ml human GM-CSF and 1000 U/ml human IL-4 (both from Gentaur). After 3 days, one third of the media was removed and spun down, and the pellet resuspended in the same volume of fresh media containing 3000 U/ml of each cytokine, then returned to the culture. After 5 days, the cells were frozen following washing with HBSS/2% FCS, by resuspending in 5.5% hetastarch (“Voluven” from John Radcliffe Hospital pharmacy)/4.8% DMSO (Hybrimax grade from Sigma)/3.8% FCS in isotonic saline, then placing in a controlled freezing device (1 degree/minute) at −80° C.

Screening for DC Modulatory Activity

Routine screening for DC modulatory activity was performed using DC generated in FCS-containing culture and frozen, as described above. DC were thawed, then cultured in 96-well flat-bottomed Primaria plates (BD Biosciences) in DC-RPMI supplemented with the samples to be screened. After 24 hours of culture, poly(I:C) (25 m/ml) or LPS (200 ng/ml) were added to stimulate the DC, with the choice of stimulus determined by the responsiveness to different stimuli of each batch of DC. After another 18-24 hours culture levels of CD86 expression by CD1a+ cells was assessed by flow cytometry.

Flow Cytometry

Flow cytometry was performed using a FACSort flow cytometer (Becton Dickinson) controlled using CellQuest Pro (Becton Dickinson). Data analysis was performed with CellQuest Pro and with FloJo (Treestar Software).

PCR

PCR was performed using a DNAEngine thermocycler (Biorad). Primers were manufactured by MWG Biosciences to HPSF quality, and dNTPs were from Bioline. Other reagents were supplied as noted in the text.

DNA Sequencing

DNA sequencing was carried out by Geneservice Ltd., Oxford, using BigDye v3.1 chemistry.

E. coli Transformation

For heat shock transformation of chemically competent E. coli, 50 μl of bacteria were thawed on ice, incubated on ice for 5 minutes with DNA, heat shocked for 40 seconds at 42° C., then placed back on ice. 250 μl of room temperature SOC media was then added, and the cells incubated at 37° C. with 200 rpm orbital shaking for 45 minutes-1 hour 30 minutes, after which a 50 ml aliquot was spread on to an LB agar plate containing 100 m/ml ampicillin.

### Example 1

### Suppression of LPS-Induced Upregulation of CD86 by SGE

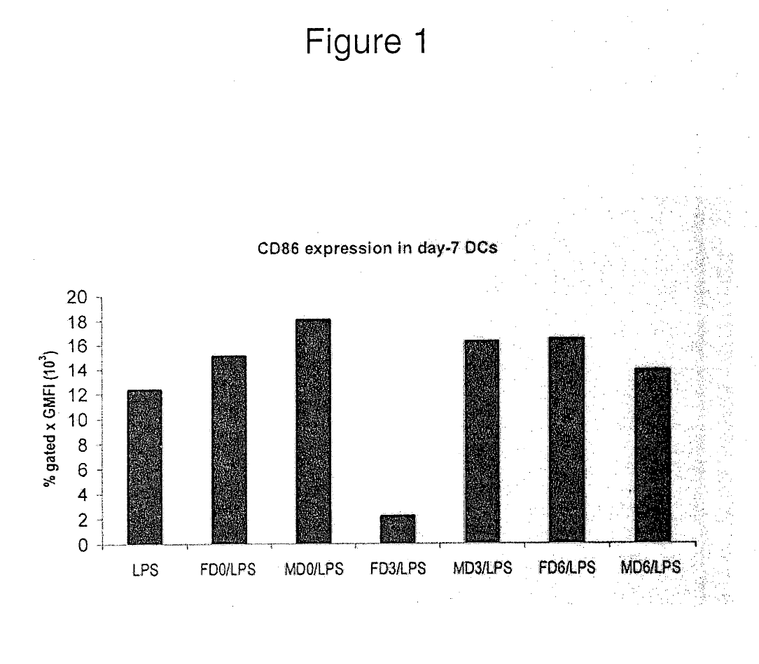

Monocytes were isolated by transient adherence and cultured with GM-CSF+IL-4 to generate DCs, as previously described. After 5 days of culture, SGE from male or female R. appendiculatus ticks was added to the cultures. The SGE was derived either from unfed ticks, or ticks that had fed for 3 or 6 days, and was added to give a final tick-derived protein concentration of 50 μg/ml. After a total of 6 days of culture, the DC were treated with LPS (50 ng/ml), and after 7 days of culture they were stained with anti-CD1a and anti-CD86 and analysed by flow cytometry. FIG. 1 shows the number of CD86⁺ cells as a % of CD1a⁺ cells×geometric mean fluorescence intensity (GMFI) of CD86 staining on these cells. As shown in FIG. 1, SGE from female Rhipicephalus appendiculatus ticks fed for 3 days, but not from unfed females, females fed for 6 days, or from male ticks (fed and unfed), was found to suppress the LPS-induced upregulation of the co-stimulatory receptor CD86 by human DCs. This experiment was performed twice, with similar results. Similar results were also obtained when the SGE was added to the culture to a final concentration of the material derived from one gland per ml.

### Example 2

### Suppression of LPS-Induced Upregulation of CD80 and MHC Class II by SGE

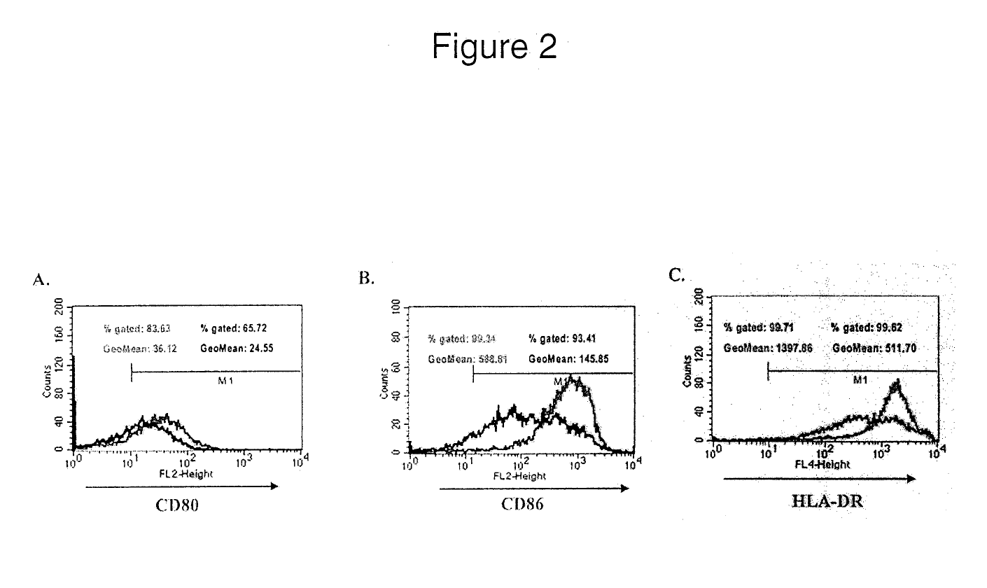

Monocytes were isolated by transient adherence and cultured with GM-CSF+IL-4 to generate DC, as previously described. After 5 days of culture, SGE from female 3 day-fed R. appendiculatus ticks was added to some cultures, to a final concentration of the material derived from one gland per ml of culture media. After a total of 6 days of culture, the DC were treated with LPS (50 ng/ml), and after 7 days of culture they were stained with anti-CD 1a, anti-CD80, anti-CD86 and anti-HLA-DR and analysed by flow cytometry. FIG. 2 shows the expression of maturation markers by CD1a⁺ cells treated with LPS in the presence (black), and absence (grey) of SGE. As shown in FIG. 2, female 3 day-fed Rhipicephalus appendiculatus salivary gland extract not only inhibits CD86-upregulation in response to LPS, but also the upregulation of CD80 and MHC Class II. This experiment was performed twice.

### Example 3

### Q column Fractionation of the Active Component of SGE

SGE derived from 112 glands from 3 day-fed female R. appendiculatus ticks was diluted 1:10 in 50 mM sodium phosphate (pH7.0), then applied to a Hi-Trap Q sepharose ion-exchange column (Amersham) which had previously been equilibrated with the same buffer. The unbound material (Q column flowthrough=QFT) was collected, the column was washed with 2 column volumes of 50 mM sodium phosphate (pH7), then bound material (Q column-bound fraction=QFR) was eluted by the application of 50 mM sodium phosphate, 1M NaCl (pH7.0) to the column. The QFT and QFR were concentrated to a final volume of 500 W using Vivaspin 6 5kDa MWCO centrifugal concentrators (Sartorius) which had been pre-treated with γ-globulin to prevent non-specific absorbance of proteins.

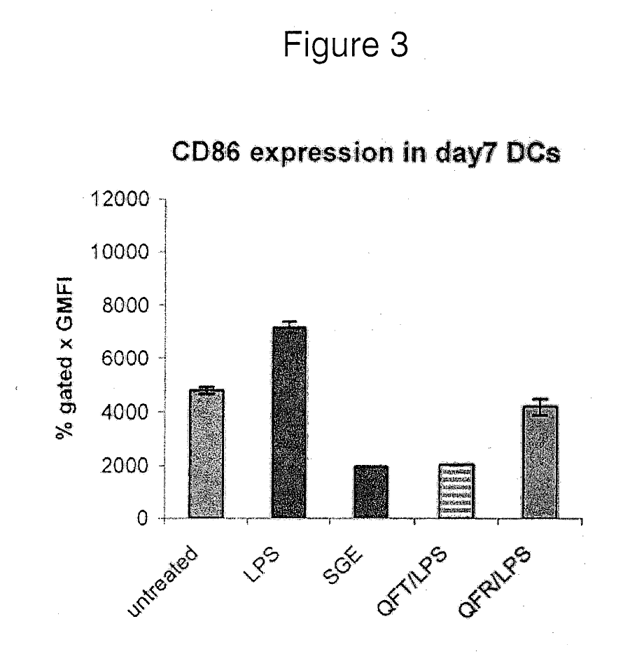

Monocytes were isolated by transient adherence and cultured with GM-CSF+IL-4 to generate DC, as previously described. After 5 days of culture, SGE from female 3 day-fed R. appendiculatus ticks, or QFT or QBF generated as described above, was added to the cultures. The final concentration of each provided material derived from one gland per ml of culture media. After a total of 6 days of culture, the DC were treated with LPS (50 ng/ml), then after 7 days of culture they were stained with anti-CD 1 a and anti-CD86 and analysed by flow cytometry. FIG. 3 shows the number of CD86⁺ cells as a % of CD1a⁺ cells×geometric mean fluorescence intensity (GMFI) of CD86 staining on these cells. The error bars represent the range between duplicate cultures.

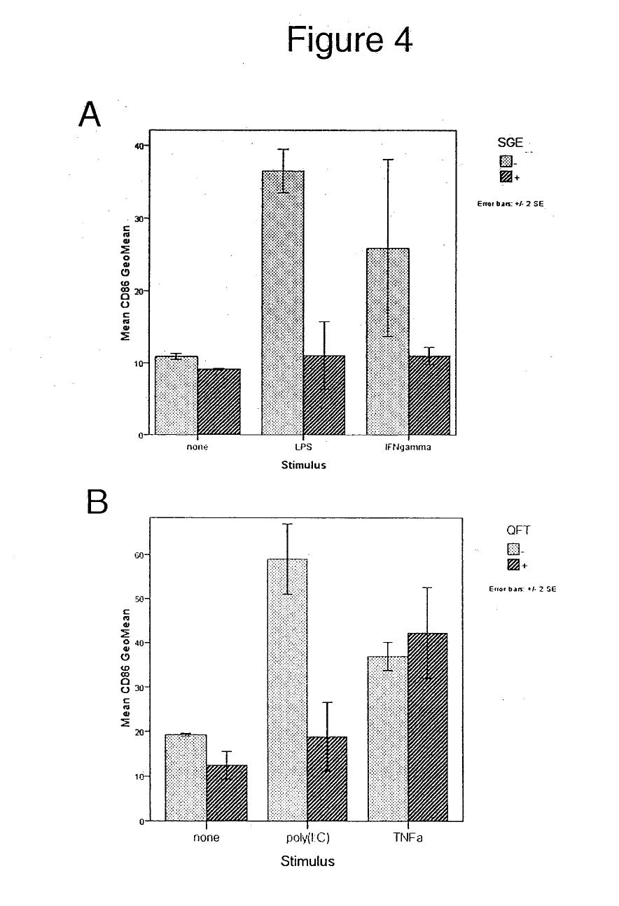

FIG. 4 shows that the DC-modulatory activity was not restricted to LPS-induced maturation (presumably acting through TLR4), but also inhibited CD86 upregulation in response to treatment with 25 μg/ml poly(I:C) (a TLR3 ligand) or with 50 ng/ml IFN-γ (FIG. 4a). However, QFT has little or no effect on 100 ng/ml TNF-α-induced CD86 upregulation (FIG. 4b). The reasons for this are not at present clear.

### Example 4

### Effect of Proteinase K on the DC Modulatory Activity of QFT

Frozen day 5 DC prepared as described above were thawed and cultured with QFT at a final concentration of 0.2 gland equivalents/ml. In this experiment, some of the QFT was pre-treated with Proteinase K, as described below. One day later, the DC were treated with poly(I:C) (25 μg/ml), then after a further day of culture they were stained with anti-CD1a and anti-CD86 and analysed by flow cytometry.

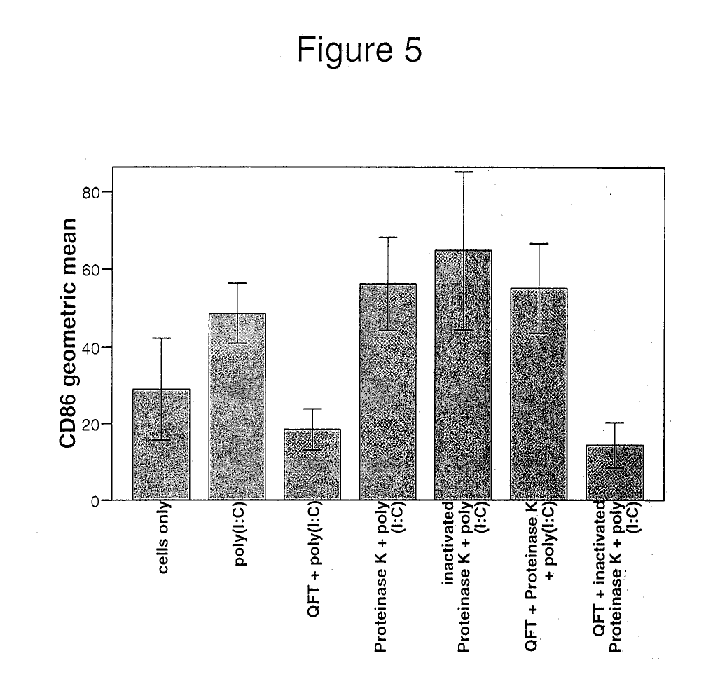

For Proteinase K treatment, QFT was incubated at 50° C. with 150 μg/ml Proteinase K (Sigma) in 50 mM Na₂HPO₄ (pH7.0) for 2 hours. The enzyme was then inactivated by heating to 98° C. for 10 minutes. A protease-only control [Proteinase K+poly(I:C)] was performed using the product of an otherwise identical reaction performed without QFT, and the QFT used in a protease-free control [QFT+poly(I:C)] was treated in the same way to demonstrate that the abrogation of DC-modulatory activity was not due to heat denaturation of the active component. Two further controls were performed in which heat inactivated Proteinase K was incubated with or without QFT [inactivated Proteinase K+poly(I:C)] and [QFT+inactivated Proteinase K+poly(I:C)], in order to confirm that the (heat labile) proteolytic capacity of Proteinase K was required for the abrogation of DC-modulatory activity. For these, Proteinase K was pre-treated for 10 minutes at 98° C. prior to the 50° C. incubation. FIG. 5 shows that treatment with proteinase K abrogates the DC modulatory ability of QFT, confirming its proteinaceous nature. Results are the mean of duplicate wells ±2 S.E.

### Example 5

### Size Fractionation of QFT

350 salivary glands were dissected from 3 day-fed female R. appendiculatus ticks, and used to prepare QFT as previously described. This QFT was size fractionated by gel filtration with a Superdex 75 column in 8 separate experiments. In each case, ˜40 fractions were collected from the column and screened for DC-modulating activity by culture with thawed day 5 DC, as previously described. The DC were stimulated by the addition of 200 ng/ml LPS after 24 hours, and the CD86 expression of CD1a⁺ cells was assessed by flow cytometry after a further 24 hours.

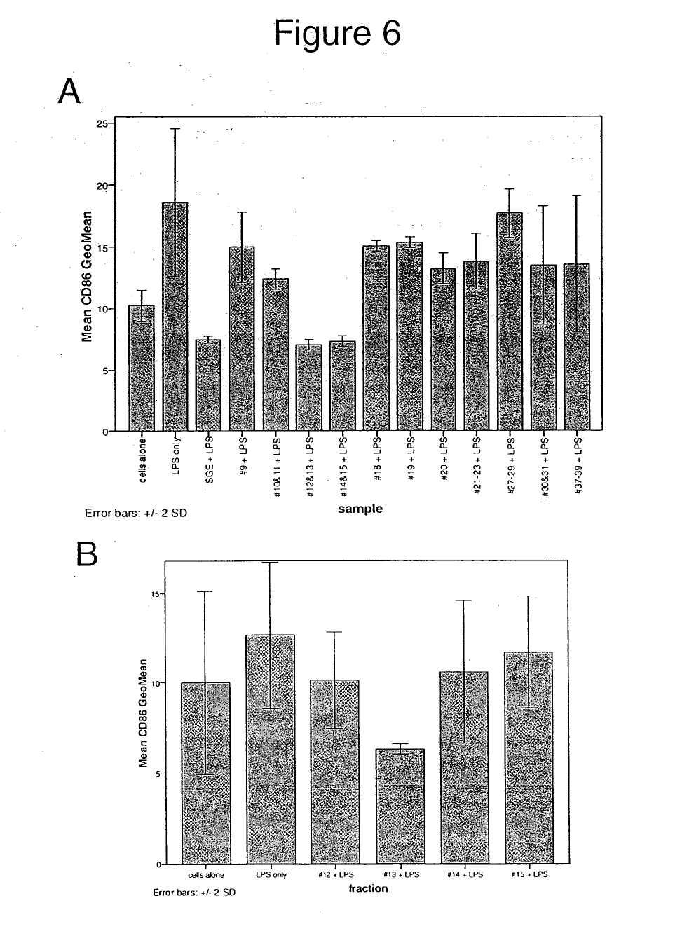

Results from one representative fractionation out of 8 are shown in FIG. 6, with an initial screen at 1 gland equivalent/ml, and a subsequent screen of putative active fractions at 0.04 gland equivalents/ml. In all fractionations, fraction #12 or #13 was identified as containing the most activity.

The most active fractions from each fractionation were pooled and dialysed against a low salt buffer [50 mM HEPES, pH 8.3], then run on a 4-12% Bis-Tris polyacrylamide gel (Invitrogen) under reducing conditions. SGE, QFT and two Q column-bound fractions were run alongside the pooled active fractions, for the purposes of comparison.

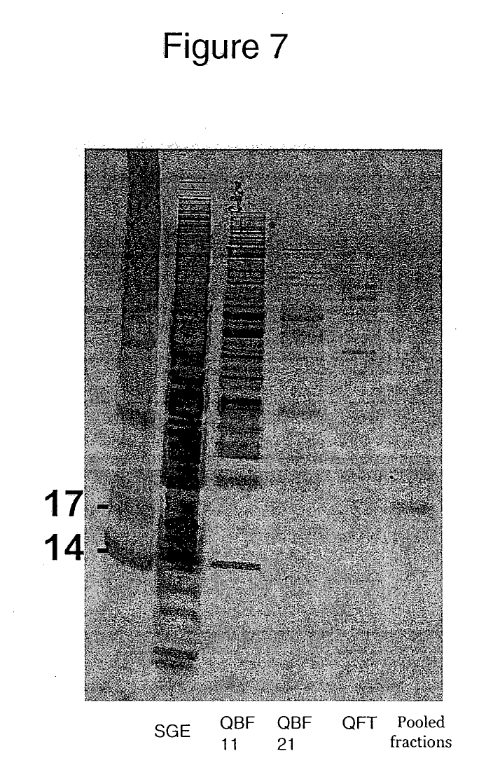

The results are shown in FIG. 7. SGE and QFT possess DC-modulatory activity, while the Q-bound fractions do not. The presence of a band at around 20 kDa in the QFT and in the pooled fractions, but not in the two inactive samples, suggests that this band represents the active protein. The inventors have named this protein “Japanin”.

The most active fractions obtained from gel filtration chromatography of QFT (see above) were pooled, dialysed against a low salt buffer, then fractionated using HPLC with a C4 column. 23 fractions were obtained from the column by elution with an increasing gradient of acetonitrile, and screened for DC modulating activity by culture with thawed day 5 DC, as previously described. The DC were stimulated by the addition of 25 μg/ml LPS after 24 hours, and the CD86 expression of CD1a⁺ cells was assessed by flow cytometry after a further 24 hours.

### Example 6

### Isolation of the DC Modulatory Activity Using HPLC

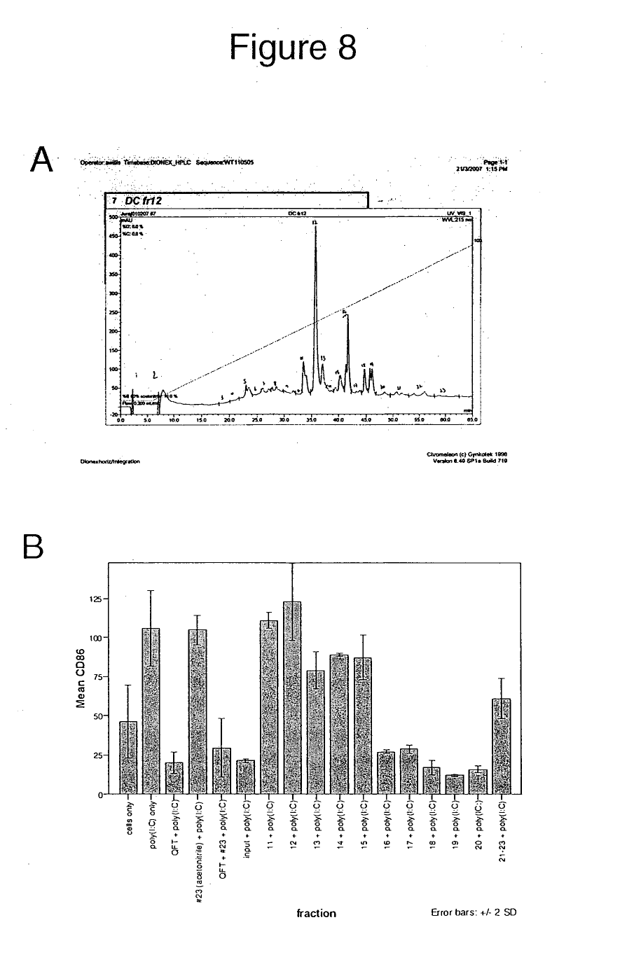

Each fraction was assessed at a dilution of 1:200, resulting in a gland equivalents/ml concentration of between 2.6 and 7.9, depending on the volume of the fraction, while the inputted material (pooled active gel filtration fractions) and QFT were included as controls, at 1.8 and 0.2 gland equivalents/ml, respectively. As fraction #23 was ˜100% acetonitrile, its effect was assessed with and without the presence of QFT in order to rule out a direct DC cell-modulatory effect of the HPLC solvent, or, conversely, the ability of the solvent to mask such an effect. FIG. 8 shows the results of the HPLC. FIG. 8a shows the elution profile, and FIG. 8b shows the activity relating to each fraction, which was assessed as inhibition of upregulation of DC86 by DCs.

### Example 7

### Edman Degradation

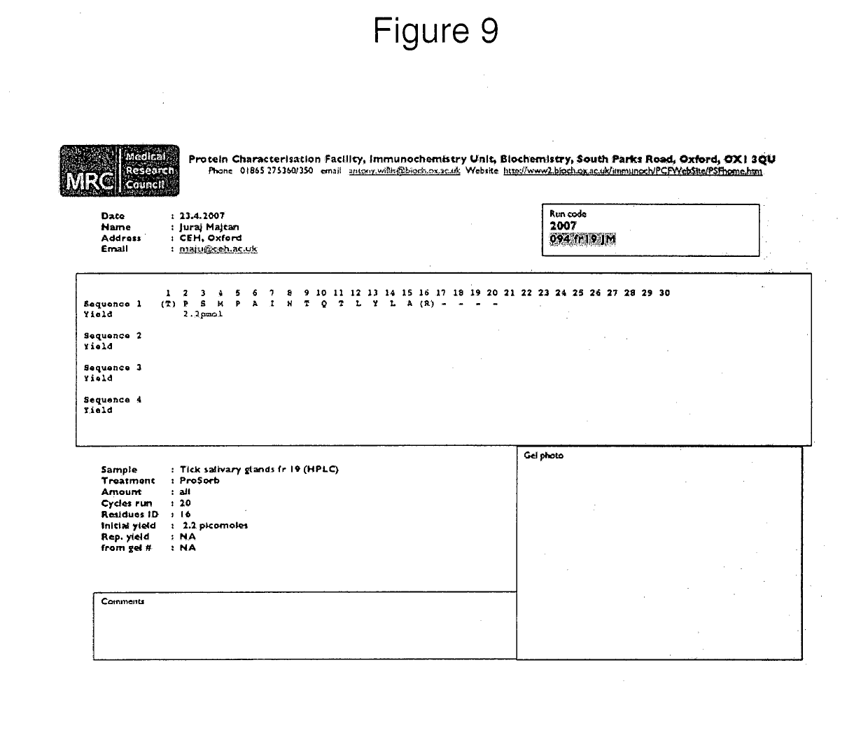

Following the characterisation of HPLC fraction #19 as possessing the most potent DC-modulatory activity, it was used for Edman degradation sequencing, generating a 16-residue N-terminal sequence: (Thr) Pro Ser Met Pro Ala Ile Asn Thr Gln Thr Leu Tyr Leu Ala (Arg), where the identification of residues in parenthesis is tentative. The Edman degradation readout is shown as FIG. 9. The protein with this N-terminal sequence is henceforth referred to as “Japanin”.

### Example 8

### PCR Amplification of the 3′ Region of Japanin DNA

The N-terminal sequence was used to design external forward primers for PCR amplification of Japanin DNA in combination with a poly(dT) reverse primer. A set of 4 degenerate primers against the sequence “M P A I N T Q” was employed. These 4 are very similar, but used separately to reduce degeneracy:

External Primer 1
(SEQ ID NO: 13)
ATG CCN GCN ATC AAY ACN CAA
External Primer 2
(SEQ ID NO: 14)
ATG CCN GCN ATC AAY ACN CAG
External Primer 3
(SEQ ID NO: 15)
ATG CCN GCN ATW AAY ACN CAA
External Primer 4
(SEQ ID NO: i6)
ATG CCN GCN ATW AAY ACN CAG

To provide a template for PCR, cDNA was generated from I day-fed female R. appendiculatus salivary glands. RNA was isolated from 30 salivary glands using Trizol reagent (Invitrogen) in accordance with the manufacturer's instructions, then precipitated from aqueous phase by addition of ⅓ volumes 8M Lithium chloride. Following washing with cold 75% ethanol, the RNA was redissolved in 5 μl of RNase-free water.

Reverse transcription was performed in a 40 μl reaction using the ImPromII reverse transcriptase (Promega), in accordance with the manufacturer's instructions. The reaction contained 4 μg of RNA, and had an MgCl₂ concentration of 2.5 mM and a total dNTP concentration of 0.5 mM. Oligo(dT)₁₅ served to prime the reverse transcription, and was incorporated at 0.1 μg/ml. The reaction was performed at 42° C. for 1 hour, and was followed by a 15 minute heat inactivation at 70° C.

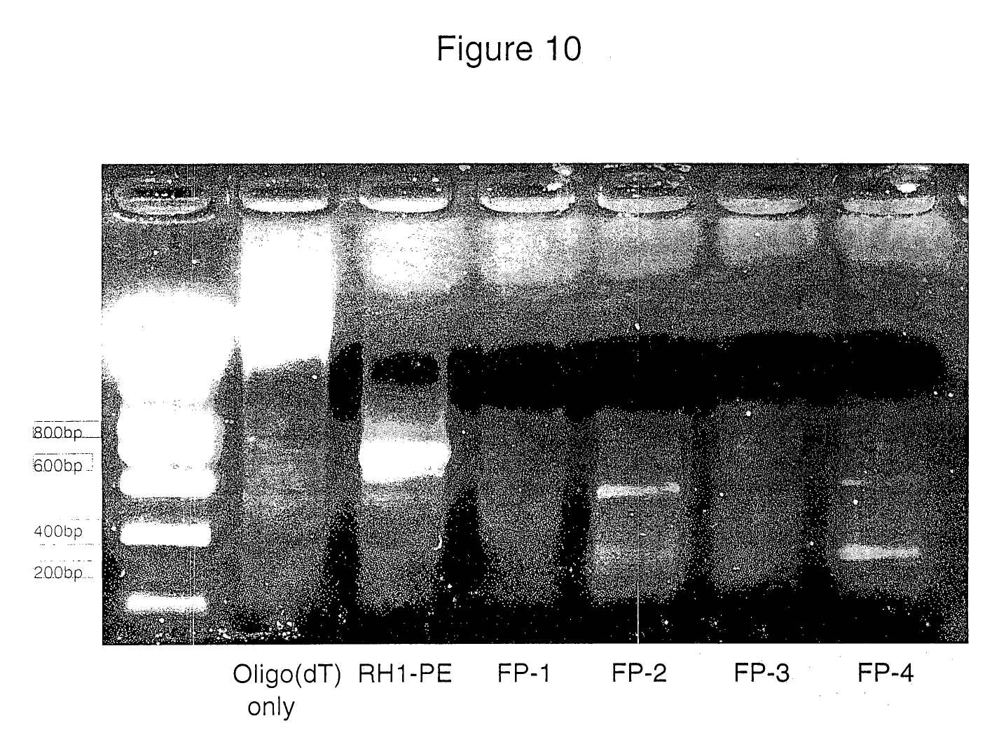

PCR using this cDNA was performed with Taq DNA polymerase (New England Biosciences) in 1×Thermopol buffer (New England Biosciences) supplemented with 62.5 μM each dNTP, 250 nM degenerate primer and 3.25 μM Oligo(dT)₂₀-V. The template cDNA was used at a dilution of 1:40. An initial denaturation step of 1 minute at 94° C. was followed by 5 cycles of 30s @ 94° C. [denaturation] / 30s @ 45° C. [annealing]/60s @ 72° C. [extension] then 30 cycles of @ 94° C. / 30s @ 50° C./60s @ 72° C. and finally an additional 5 minutes @ 72° C. A positive control was performed using a forward primer for a known tick protein under the same conditions [RH1-PE]. The products were run on a 1.8% agarose gel. The results are shown in FIG. 10.

The larger products amplified by forward primers 2 and 4 were ˜600 bp, suggesting that they encode a protein of ˜22 kDa (given an average amino acid residue mass of 110 Da). This corresponds well to the ˜20 kDa band identified in active fractions on an SDS-PAGE gel. Before cloning this cDNA, we proceeded to confirm that it corresponded with the N-terminal sequence by carrying out nested PCR.

Internal primers were designed against the sequence “Asn Thr Gln Thr Leu Tyr Leu Ala”—this is within the N-terminal sequence but downstream of the binding site for the primers used previously:

Internal Primer 1
(SEQ ID NO: 17)
GCY ACI CAG ACI YTI TAY CTN GC
Internal Primer 2
(SEQ ID NO: i8)
GCY ACI CAG ACI YTI TAY TTR GC

The non-standard code “I” signifies the incorporation of inosine as a neutral base.

Template was provided by DNA amplified with the primer encoded by forward primers 2 and 4, along with Oligo(dT)₂₀-V, used at a 1:20 dilution. Reaction conditions were as described above, except that all 35 cycles used an annealing temperature of 50° C., and that the 72° C. extension step was shortened from 60s to 40s. Products were run on a 1.8% agarose gel.

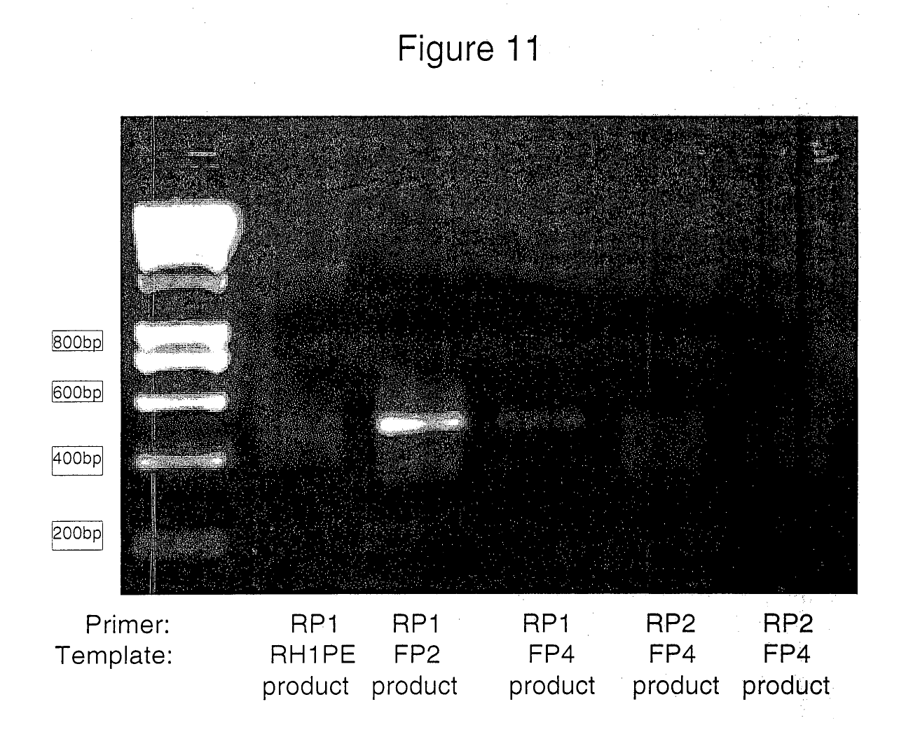

These PCRs produced bands of the expected size (i.e. ˜600 bp), as shown in FIG. 11, strongly suggesting that the cDNA encoding the N-terminal protein sequence is being specifically amplified.

### Example 9

### Identification of a Consensus Sequence for the 3′ Region of Japanin cDNA

In order to obtain the 3′ sequence of Japanin, DNA was amplified using the external forward primers 2 and 4, cloned into pCR2.1, and sequenced.

External forward primers 2 and 4 were employed in combination with Oligo(dT)₂₀-V as a reverse primer, with 40 μl reactions being performed as previously described. The reactions were run on an agarose gel, and the ˜600 bp DNA bands excised and then purified using the QIAquick gel extraction kit (Qiagen) in accordance with the manufacturer's instructions, with elution from the column with 30 μl of elution buffer.

6 μl of each purified product was ligated into pCR2.1 by incubating overnight at 14° C. with 50 ng of pCR2.1-TA (Invitrogen) and T4 DNA Ligase (NEB Biosciences), in a 10 μl reaction. The ligation mixtures were used to transform competent TOP 10 strain E. coli, and insert-containing colonies were identified by PCR screening using forward primers 2 and 4 in combination with Oligo(dT)₂₀-V. Insert-containing pCR2.1 DNA was isolated from 5 ml cultures of positive colonies using the QIAprep Spin kit (Qiagen) in accordance with the manufacturer's instructions.

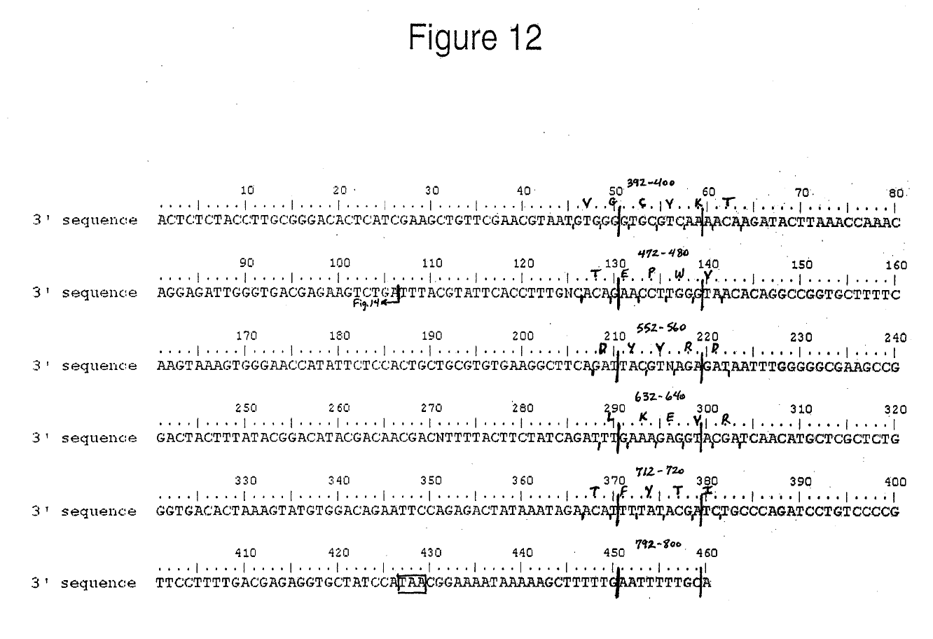

Sequencing of four insert-containing plasmids allowed the construction of a consensus sequence for the 3′ region of Japanin cDNA, which is shown in FIG. 12.

### Example 10

### Sequencing the 5′ Region of Japanin

A 5′RACE (Rapid Amplification of cDNA Ends) strategy was employed to amplify a ˜500 bp product incorporating the 5′ region of Japanin.

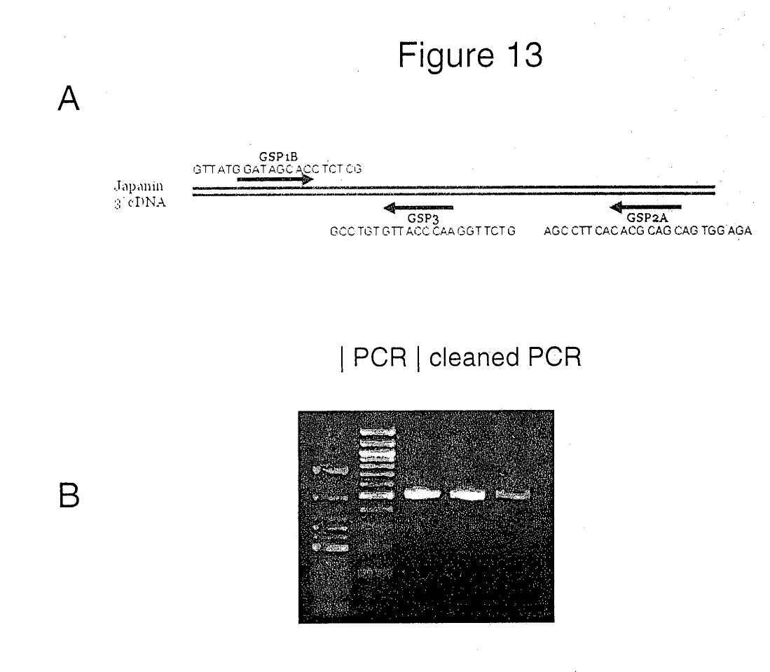

5′RACE utilises a known 3′ cDNA sequence (in this case the newly-obtained Japanin 3′ sequence) to inform the design of gene-specific primers. These primers are used to perform gene-specific reverse transcription and to amplify the 5′ region of cDNA using nested PCR. In the latter case, the forward primers are specific for an experimentally-introduced oligonucleotide cap region, while the reverse primers are gene-specific reverse primers. The relative positions of the various Japanin-specific primers used are shown in FIG. 13a.

RNA was extracted from salivary glands of 2-day fed female R. appendiculatus ticks using the RNAqueous-4PCR kit (Ambion) in accordance with the manufacturer's instructions. The RNA was precipitated from the column eluate and redissolved in 200 of elution buffer.

Gene-specific reverse transcription was performed using the GSP1B primer. The ImProm II RT enzyme (Promega) was employed in a 20 μl reaction containing 1 μg of RNA, in accordance with the manufacturer's instructions. MgCl₂ concentration in the reaction was 2.5 mM, total dNTP concentration was 0.5 mM and primer concentration was 125 nM. The RT reaction was performed at 48° C. for 1 hour, and was followed by a 15 minute heat inactivation at 70° C. RNA was then removed by addition of 1 μl RNase mix (Invitrogen, from the 5′RACE System kit) and incubation at room temperature for 30 minutes.

The generated cDNA was cleaned to remove enzymes, primers and nucleotides using a SNAP column (Invitrogen, from the 5′RACE System kit) in accordance with the manufacturer's instructions. cDNA was eluted in 50 μl of nuclease-free water.

15 μl of cDNA was tailed with oligo(dC) in a 25 μl reaction, using the TdT enzyme and dCTP (Invitrogen, from the 5′RACE System kit) in accordance with the manufacturer's instructions.

1 μl of poly(dC)-tailed cDNA was used in a 20 μl reaction as a template for nested PCR. The first round of amplification was performed using the GSP2A primer in combination with the AAP primer (Invitrogen, from the 5′RACE System kit), and the product was gel purified and used as the template in the second round of amplification, performed using the GSP3 primer in combination with the AUAP primer (Invitrogen, from the 5′RACE System kit).

Both PCRs were performed using Taq DNA polymerase (New England Biosciences), using 1×Thermopol buffer New England Biosciences) containing 2 mM Mg²⁺ and supplemented with 62.5 μM each dNTP (Bioline) and 250 nM each primer. The GSP2A/AAP PCR comprised an initial denaturation step of 1 minute at 94° C., followed by 35 cycles of 30s at 94° C./30s at 66° C./40s at 72° C., and finally an additional 5 minutes at 72° C. The product of a 20 μl reaction was run on an agarose gel and a ˜650 bp band was excised and extracted using the QIAquick gel purification kit (Qiagen) in accordance with the manufacturer's instructions. The purified product was used as the template for the second round of amplification at a dilution of 1:1000.

The GSP3/AUAP PCR was similar, except that an annealing temperature of 68° C., rather than 66° C., was employed. In order to obtain sufficient DNA for sequencing, a 1500 GSP3/AUAP PCR was performed. 75 μl of the product was run on an agarose gel, and a ˜500 bp band was excised and extracted using the QIAquick gel purification kit (Qiagen) in accordance with the manufacturer's instructions. The DNA was eluted from the column in 30 μl elution buffer, and samples were run on a gel alongside unpurified PCR product to confirm recovery and to estimate concentration at ˜100 ng/μl, prior to dispatch of the remainder for sequencing. as shown in FIG. 13b.

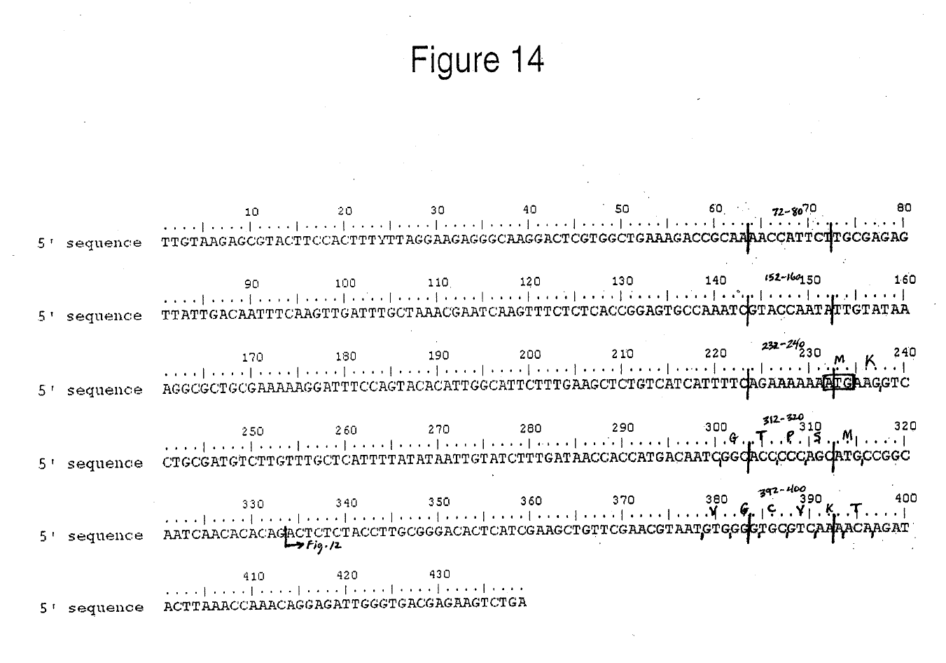

The gel purified PCR product was sequenced using the AUAP primer, yielding the 5′ sequence of Japanin cDNA, which is shown in FIG. 14.

(SEQ ID NO: 29)
GSP1B = GTT ATG GAT AGC ACC TCT CG
(SEQ ID NO: 30)
GSP2A = AGC CTT CAC ACG CAG CAG TGG AGA
(SEQ ID NO: 31)
GSP3 = GCC TGT GTT ACC CAA GGT TCT G

### Example 11

### Primer Design for Cloning Full Length Japanin

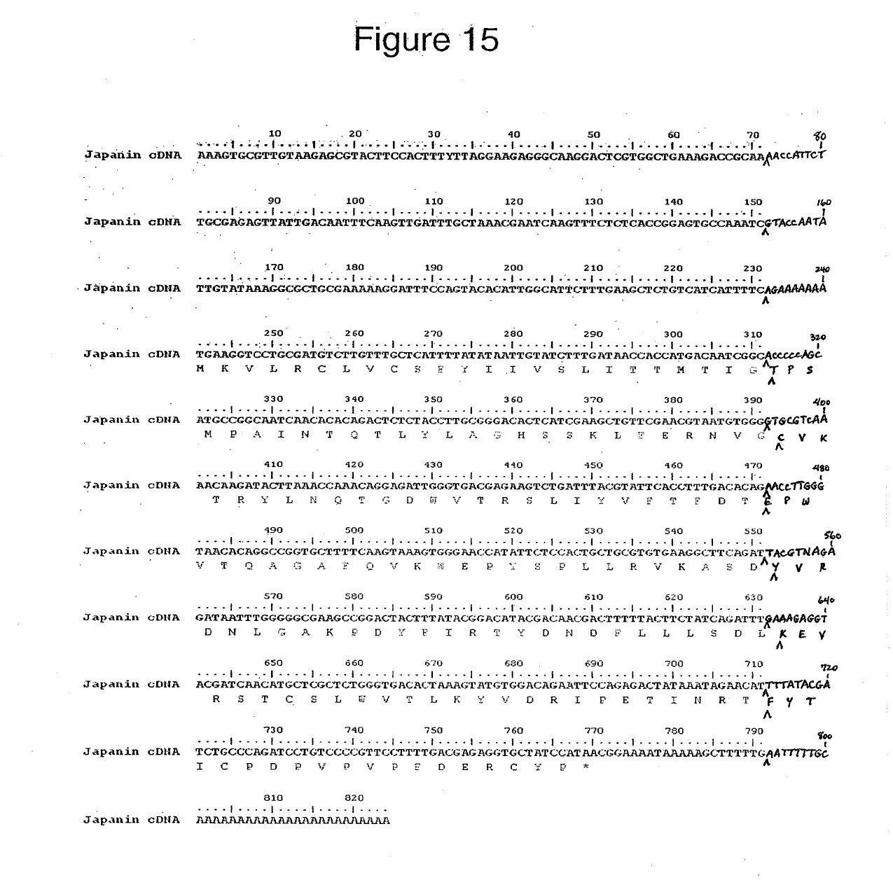

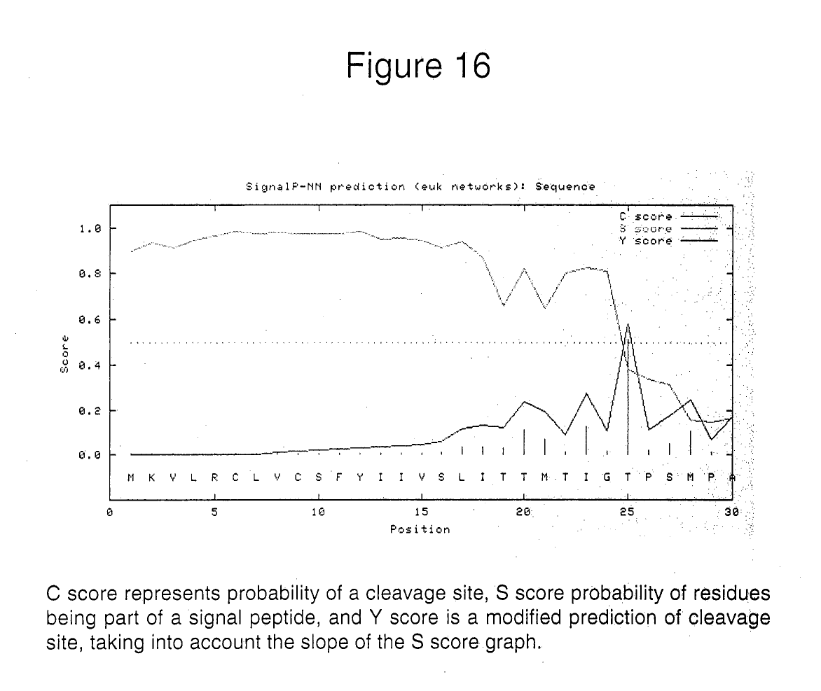

The successful cloning of the 5′ and 3′ cDNA sequences allowed the assembly of a putative full-length sequence of Japanin cDNA, encoding a 176 residue peptide, which is shown in FIG. 15. The residue positioning in the Japanin sequence and sequence similarities with other known molecules suggests that Japanin is a lipocalin.

Neural network analysis suggests that the first 24 amino acids are a signal sequence for secretion. They are followed by the sequence: Thr Pro Ser Met Pro Ala Ile Asn Thr Gln Thr Leu Tyr Leu Ala, matching the N-terminal sequence obtained from the DC-modulatory HPLC fraction. This confirms that the correct cDNA had been sequenced.

Cloning of the full-length cDNA was performed using a nested PCR strategy. Both rounds of PCR used a high fidelity DNA polymerase, in order to minimise the introduction of polymerase-generated mutations. Primers were designed based on the putative full-length cDNA sequence, including the signal sequence, with the 2^{nd} round primers incorporating BamHI and NotI restriction sites at the 5′ end of the forward and reverse primers, respectively, in order to facilitate subcloning. The restriction sites were preceded by 5 extra nucleotides, as restriction enzymes are reported to be inefficient at cutting at the very end of linear nucleic acids.

Forward primer 5
(SEQ ID NO: 19)
TGGCATTCT TTGAAGCTCTGTCATCA
Reverse primer 3
(SEQ ID NO: 20)
GCTTTTTATTTTCCGTTATGGATAGCACCTC
Forward primer 6
(SEQ ID NO: 21)
CGTTAGGATCCGGCATTCTTTGAAGCT C
Reverse primer 4
(SEQ ID NO: 22)
GTTTAGCGGCCGCCGTTATGGATAGCA

Both rounds of PCR were performed using Phusion HotStart DNA polymerase (New England Biosciences), in 1×HF buffer (New England Biosciences) supplemented with 50 μM each dNTP (Bioline) and 250 nM each primer.

The 1^{st} round of PCR was performed using the forward primer 5 and reverse primer 3, with template was provided by cDNA generated from 1 day-fed female R. appendiculatus salivary glands, as previously described. An initial denaturation step of 30s at 98° C. was followed by 35 cycles of 10s at 98° C./30s at 64.6° C./30s at 72° C., and then by an additional 1 minute at 72° C. The product of this reaction was used as the template for the second round of amplification at a dilution of 1:100000.

The 2^{nd} round of PCR was performed using the forward primer 6 and reverse primer 4. An initial denaturation step of 30s at 98° C. was followed by 2 cycles of 10s at 98° C./30s at 41° C./30s at 72° C., 20 cycles of 10s at 98° C. / 30s at 72° C. and then by an additional 5 minutes at 72° C.

### Example 12

### Cloning of Japanin

DNA encoding full-length Japanin, with the addition of a 5′ BamHI site and a 3′ NotI site was amplified by PCR as previously described, cut with BamHI and NotI, and ligated into similarly-treated pBacPAK8 vector. Ligated DNA was used to transform TOP10 E. coli, after which individual colonies were expanded and mini-prepped, and their plasmid DNA sequenced.

A 50 μl reaction to amplify DNA encoding full-length Japanin (with a 5′ BamHI site and a 3′ NotI site) was performed as previously described. 35 μl of the product was treated with the QIAquick PCR purification kit (Qiagen) in order to remove primers and nucleotides, and the plasmid was eluted in 30 μl of elution buffer diluted to 0.33× with water to reduce its buffer strength and subsequent impact on restriction enzyme buffer pH.

The amplified DNA was digested with BamHI and NotI, with a 1 hour incubation at 37° C. in BSA-supplemented 1×BamHI buffer. The enzymes were then removed by cleaning up the DNA with the QIAquick PCR purification kit (Qiagen), eluting with 30 μl of elution buffer. All enzymes and buffers were from New England Biosciences.

pBacPAK8 plasmid was similarly digested, then gel purified using the QIAquick gel extraction kit in order to ensure removal of the excised multiple cloning site fragment.

The Japanin DNA was ligated into the pBacPAK8 in a 10 μl reaction containing ˜60 ng cut pBacPAK8 and ˜5 ng cut Japanin PCR product, with 1 μl DNA Ligase (New England Biosciences) in 1×T4 DNA Ligase buffer (New England Biosciences).

3 μl of the ligation reaction was used to transform 50 μl of chemically competent TOP10 E. coli. Following overnight culture on LB agar supplemented with ampicillin, isolated colonies were used to inoculate 5 ml LB media (+ampicillin) overnight cultures, from which plasmid DNA was isolated using the QIAprep Spin kit (Qiagen) in accordance with the manufacturer's instructions.

Sequencing was performed using the Bac1 and Bac2 primers. The sequences obtained confirmed the accuracy of the putative complete cDNA sequence, and allowed selection of a mutation-free clone for generation of recombinant baculovirus (pBacPAK8-Japanin).

### Example 13

### Expression of Japanin in Insect Cell Culture

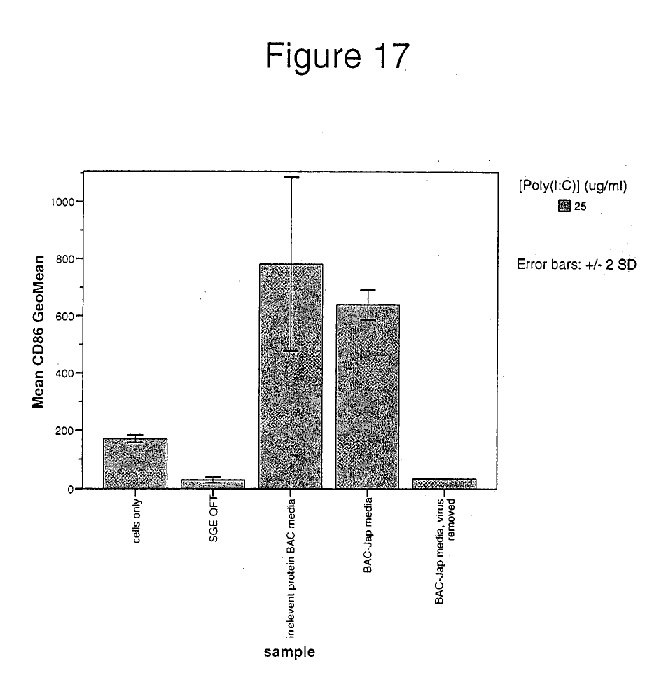

Japanin-expressing recombinant baculovirus was generated using the flashBac system, whereby Sf9 insect cells are co-transfected with Japanin transfer vector and mutant virus with a defective essential gene. Homologous recombination between the vector and the virus restores function of the essential gene while simultaneously inserting the Japanin sequence into the virus, under the control of a strong promoter. This ensures that all viable virus contains Japanin DNA. Following amplification, recombinant virus was used to infect fresh Sf9 cells, the culture supernatant from which was collected and screened for DC-modulatory activity. As shown in FIG. 17, the supernatant was found to possess DC modulatory activity, demonstrating that functional Japanin was produced and secreted into the media, though in order to unmask this activity, it was necessary to first remove the viral particles by passing the supernatant through a 100 kDA MWCO filter, presumably because the highly stimulatory effects of the virus particles overwhelmed or bypassed the inhibitory effects of Japanin.

Log.-phase Sf9 cells were allowed to adhere to a 6-well plate at a density of 1.1×10⁶ cells/well, then transfected with a mixture of flashBac gold baculovirus DNA (Oxford Expression Technologies) and pBacPAK8-Japanin using the Cellfectin transfection reagent (Invitrogen). 500 ng of plasmid DNA was mixed with 0.5 μl of flashBac gold DNA and 5 μl of Cellfectin in 1 ml of Sf900 II media, and incubated for 25 minutes at room temperature to allow complexes to form. The media was removed from the adherent Sf9 cells and replaced with the DNA/Cellfectin complexes and the cells incubated overnight, after which a further 1 ml of Sf900 II media was added and the incubation continued for a further 4 days. At this point the virus-containing supernatant was harvested and stored in the dark at 4° C.

The small volume of viral stock obtained in this way was then used to seed a larger culture of Sf9 cells to amplify the virus. 0.5 ml of the virus-containing supernatant was added to a 250 ml shake culture of log.-phase Sf9 cells (in which the cells were at a density of ˜1.5×10⁶/ml). The cultures were then incubated for 5 days, after which the supernatant was harvested and stored in the dark at 4° C.

The amplified viral stock was used to infect Sf9 cells at a high multiplicity of infection in order to drive protein expression. 25 ml of viral stock was added to a 250 ml culture of log.-phase Sf9 cells (cells at 8×10⁵/ml). The cultures were then incubated for 3 days, then the supernatants harvested by centrifugation.

In order to remove viral particles, a 5 ml sample of the supernatant was passed through a 100 kDa MWCO Vivaspin 6 centrifugal concentrator (Sartorius). This sample was screened for DC-modulatory activity in the usual way, alongside QFT (as a positive control), unfiltered baculovirus/Japanin supernatant, and supernatant from a baculovirus-Sf9 cell culture expressing an irrelevant protein. The results clearly show the presence of DC modulatory activity in the supernatant, although it is masked by the presence of viral particles in the unfiltered supernatant, perhaps because the particles themselves deliver an overwhelming stimulus to the DC. This result confirms that the protein cloned as “Japanin” does indeed possess the predicted DC-modulatory properties, and that it is produced in an active form by Sf9 cells.

### Example 14

### Precipitation of Proteins Isolated From the Supernatant of Japanin Containing Insect Cells

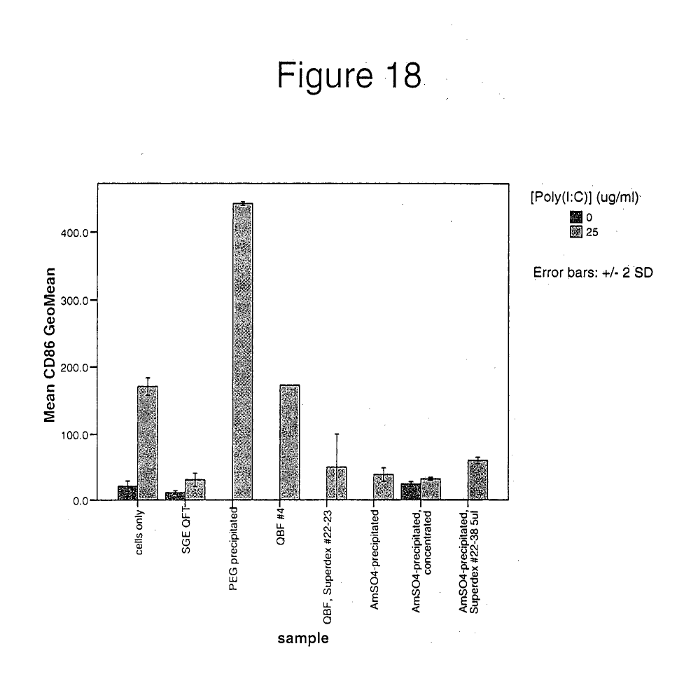

Protein was precipitated from baculovirus/Japanin supernatant by addition either of polyethylene glycol (PEG) or ammonium sulphate. The ammonium sulphate-precipitated protein was further fractionated by gel filtration using a Superdex 75 column. As shown in FIG. 18, DC-modulatory activity was found to be retained in the ammonium sulphate-precipitated but not the PEG-precipitated proteins, and was also present in pooled Superdex fractions #22-38 (which contained the majority of the protein). Subsequent screening of fractions #22-38 revealed that pooled fractions #23+24 were the most active.

PEG was gradually added to supernatant to a final level of 30% (w/v), on ice, with constant stirring. Ammonium sulphate was added to 70% (w/v) in the same manner.

The PEG-precipitated protein was redissolved in 50 mM HEPES (pH7.2) and fractionated using a Q column. Real-time plotting of A₂₈₀ indicated that fraction #4 contained a distinct protein peak, and so this fraction was further fractionation by gel filtration, using a Superdex 75 column. The bulk of protein eluted from this column in fractions #22 and #23.

The Ammonium sulphate-precipitated protein was redissolved in 50 mM HEPES (pH7.2) and concentrated ×15 using a 5 kDa MWCO Vivaspin 6 centrifugal concentrator (Sartorius). The concentrated protein was fractionated by gel filtration using a Superdex 75 column. Real-time plotting of A₂₈₀ indicated that fractions #22-28 contained the bulk of the protein.

These samples were screened for DC-modulatory activity in the usual way, each at a dilution of 1:100, revealing that the activity was retained in Ammonium sulphate-precipitated protein, but not detectable in PEG-precipitated protein. As would be expected from this, the selected Q column-bound fraction of PEG-precipitated protein did not exhibit activity, and although a slight reduction in CD86 expression was observed following incubation with Superdex fractions #22+23 from this Q-bound fraction: this result was not clear-cut, and was not explored further. Conversely, Ammonium sulphate-precipitated protein retained its activity after concentration and gel filtration chromatography, and subsequent comparison of gel filtration fractions reveals fractions #23+24 to be the most active, and therefore are postulated to contain the highest concentration of Japanin.

### Example 15

### Cloning of His-Tagged Japanin

Three-stage nested PCR was used to redone Japanin with the addition of a six residue polyhistidine fusion tag (his-tag) at the C-terminus, with pBacPAK8-Japanin providing the template. The nested PCR product was digested with restriction enzymes in order to facilitate ligation into similarly restricted pBacPAK8 plasmid. The primers were designed in order to introduce four additional residues (glutamine-glycine-glycine-serine) between the his-tag and the native protein sequence. This was in order to prevent steric hindrance due to the proximity of the his-tag to the native sequence, and also to introduce a TEV protease consensus cleavage site, potentially facilitating proteolytic removal of the tag.

Forward primer 7
(SEQ ID NO: 23)
CGTTAGGATCCGGCATTCTTTGAAGCTC
Reverse primer 5
(SEQ ID NO: 24)
ATGAGAGCCTCCTTGTGGATAGCACCTCTCG
Reverse primer 6
(SEQ ID NO: 25)
TTAGTGATGATGATGATGATGAGAGCCTCCTTG
Reverse primer 7
(SEQ ID NO: 26)
AAGTGCGGCCGCTTAGTGATGATGATG

The first stage PCR was performed with Phusion DNA polymerase (New England Biosciences) in 1×HF buffer (New England Biosciences) supplemented with 50 μM each dNTP (Bioline), 500 nM each primer and either 10 μg/μl of template plasmid. An initial denaturation step of 30 seconds at 98° C. was followed by 15 cycles of 10s @ 98° C. [denaturation]/30s @ 70° C. [annealing]/15s @ 72° C. [extension] and finally an additional 5 minutes @ 72° C. The primers employed were forward primer 7 and reverse primer 5. The product of a 20 μl reaction performed in this way was cleaned-up to remove primers and nucleotides using the QIAquick PCR purification kit, and eluted in 30 μl elution buffer.

The second stage PCRs were performed in exactly the same way, except that the annealing temperature was 69° C., and the template was provided by the cleaned first stage PCR product, diluted 1:10 in distilled water, and the reverse primer employed was reverse primer 6. The product of a 20 μl reaction performed in this way was cleaned-up to remove primers and nucleotides using the QIAquick PCR purification kit, and eluted in 30 μl elution buffer.

The third stage PCR was performed in exactly the same way as the second stage PCR, except that the template was provided by the cleaned second stage PCR product, diluted 1:10 in distilled water, and the reverse primer employed was reverse primer 7. The product of a 50 μl reaction performed in this way was cleaned up to remove primers and nucleotides using the QIAquick PCR purification kit, and eluted in 30 μl 0.5×elution buffer. Running a sample from the reaction on an agarose gel allowed the concentration of PCR product to be estimated at ˜20 ng/μl.

The cleaned third stage PCR product was digested with BamHI and NotI, with a 20 minute incubation at 37° C. in a 50 μl reaction containing 1×Buffer BamHI with BSA. Buffers and enzymes were from New England Biosciences. The reaction was cleaned up to remove enzymes and excised fragments using the QIAquick PCR purification kit, with elution in 30 μl elution buffer.

In order to give a ˜1:1 molar ratio for optimal ligation, ˜4.5 ng of the restricted product was ligated with 50 ng of previously BamHI/NotI restricted and CIP-treated pBacPAK8. The ligation was performed for 15 minutes at room temperature using T4 DNA Ligase (New England Biosciences) in a 10 μl reaction containing T4 DNA Ligase buffer (New England Biosciences). The ligation mixtures were used to transform competent TOP10 strain E. coli. Discrete colonies were used to inoculate 5 ml liquid LB cultures, and plasmid DNA isolated using the QIAprep Spin kit (Qiagen) in accordance with the manufacturer's instructions. Sequencing using the Bac1 and Bac2 primers confirmed the presence of the Japanin fusion protein-encoding insert. This plasmid is henceforth referred to as pBacPAK8-Jap-TEV-his.

### Example 16

### Isolation of His-Tagged Japanin

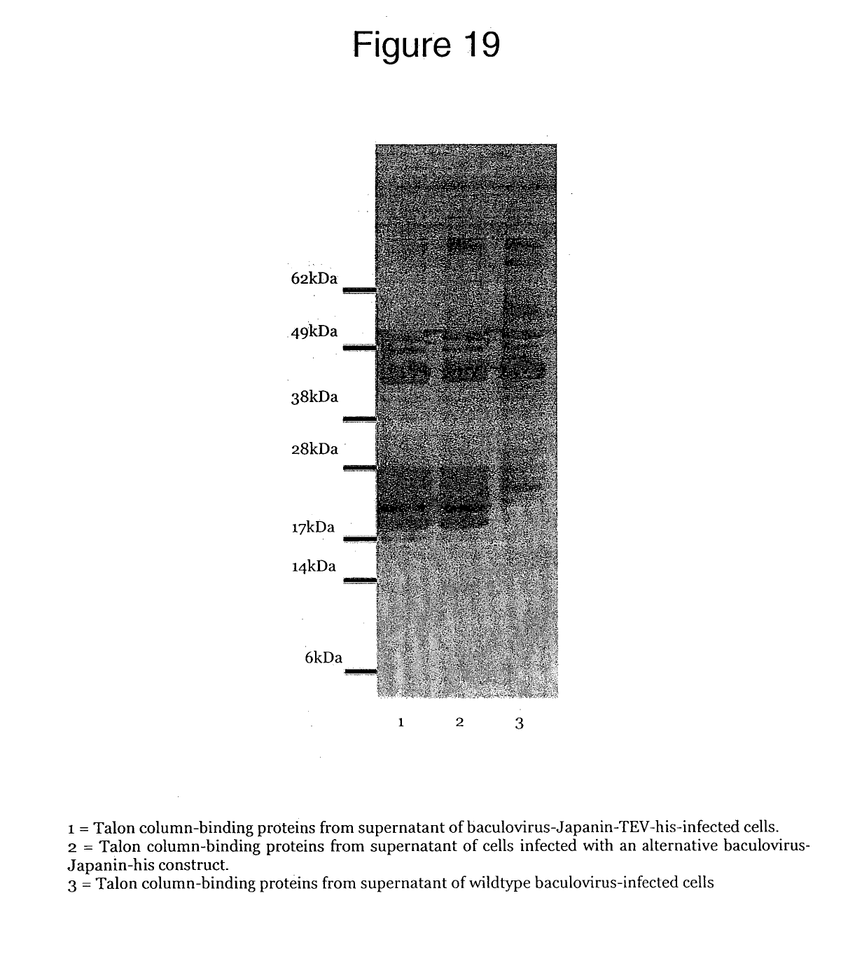

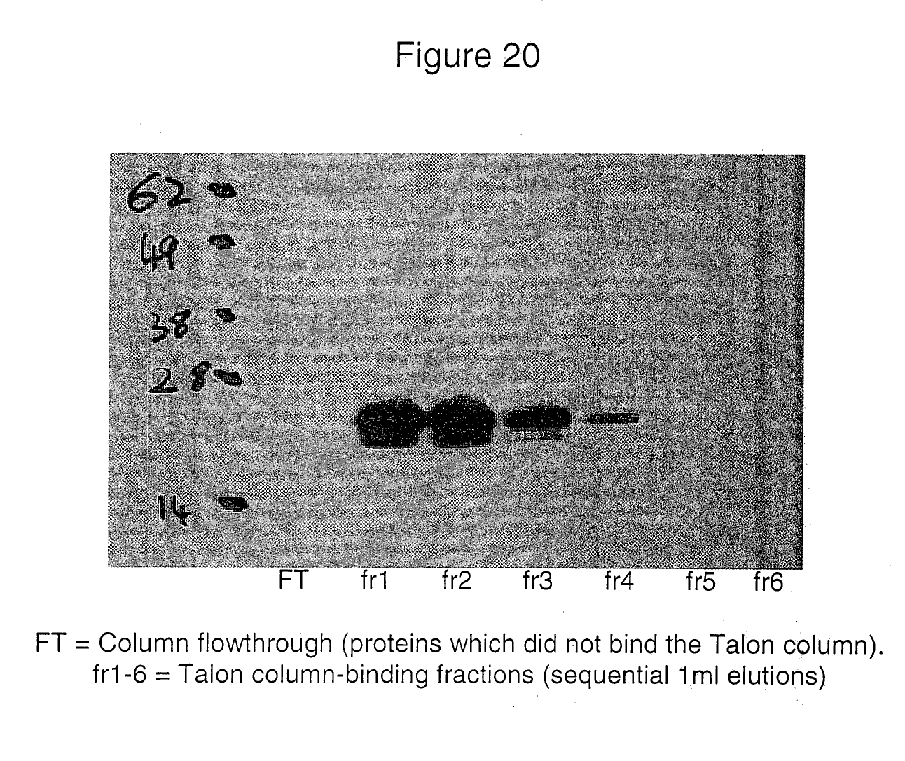

pBacPAK8-Jap-TEV-his plasmid DNA was used to generate recombinant baculovirus using the flashBac Gold system, as previously described for the production of unlabelled recombinant Japanin. These recombinant baculoviruses were then used to infect 250 ml expression cultures of Sf9 cells, again as previously described. Protein was then precipitated from culture supernatants using ammonium hydroxide, and purified using an IMAC column, which binds polyhistidine motifs. Silver staining of SDS-PAGE gels revealed the presence of a recombinant-specific protein at ˜20 kDa, as shown in FIG. 19, and that this protein is indeed his-tagged was confirmed by Western blot as shown in FIG. 20. These results demonstrate that his-tagged Japanin has been successfully expressed.

Protein was precipitated from the supernatant of 3-day 250 ml expression cultures of pBacPAK8-Jap-TEV-his by addition of ammonium sulphate to 70% (w/v), as previously described for the production of unlabelled recombinant Japanin. The precipitated protein was redissolved in 60 ml 40 mM Na₂HPO₄/300 mM NaCl/10% glycerol, pH8 (binding buffer) and loaded on to a column pre-loaded with 1 ml Talon resin (Clontech). The column was then was washed twice with 20 ml binding buffer, and then eluted with 6 ml 40 mM Na₂HPO₄/100 mM NaCl/300 mM imidazole, pH8 (elution buffer). The eluted protein was concentrated×10 with a 5000MWCO Vivaspin 6 (Sartorius).

Silver staining was performed using the SilverXpress kit (Invitrogen) in accordance with the manufacturers instructions, after 0.5 μl samples were run on a 4-12% polyacrylamide Bis-Tris gel (NuPage precast gel from Invitrogen). The presence of two major bands at ˜20 kDa is apparent in the two recombinant protein supernatants, but not in a negative control purification carried out in parallel using supernatant from cells infected with wild-type baculovirus.

For Western blot analysis, 6.5 μl samples were run on a 4-12% polyacrylamide Bis-Tris gel (NuPage precast gel from Invitrogen), then transferred to 0.45 μm-pore nitrocellulose membrane (Biorad) by wet blotting (applying a constant 30V for 1 hour in NuPage transfer buffer supplemented with 10% methanol). Once transfer was complete, the membrane was rinsed with distilled water, washed for 5 minutes with Tris buffered saline/0.1% Tween 20 (TBST) then incubated for 1 hour at room temperature in blocking reagent (#B6429, Sigma). After blocking, the membrane was rinsed briefly with TBST, then incubated overnight at 4° C. with anti-penta-his antibody (Qiagen) diluted 1:1000 in blocking reagent. This was followed by 5×five minute washes in TBST, incubation for 1 hour at room temperature in donkey-anti-mouse-HRP (Jackson Immunoresearch) diluted 1:20000 in 10% non-fat dried milk (Marvel)/TBS, and seven further 5 minute washes in TBST. Finally, the antibody was visualised by treating the membrane with ECL substrate (Amersham) in accordance with the manufacturer's instructions, and then exposing X-ray film to it for 10 seconds. The presence of a ˜20 kDa band is apparent in first four 1 ml fractions eluted from the column, confirming the presence of a his-tagged protein of the predicted size.

### Example 17

### CD modulatory Activity of Japanin-TEV-His Supernatant-Derived Samples

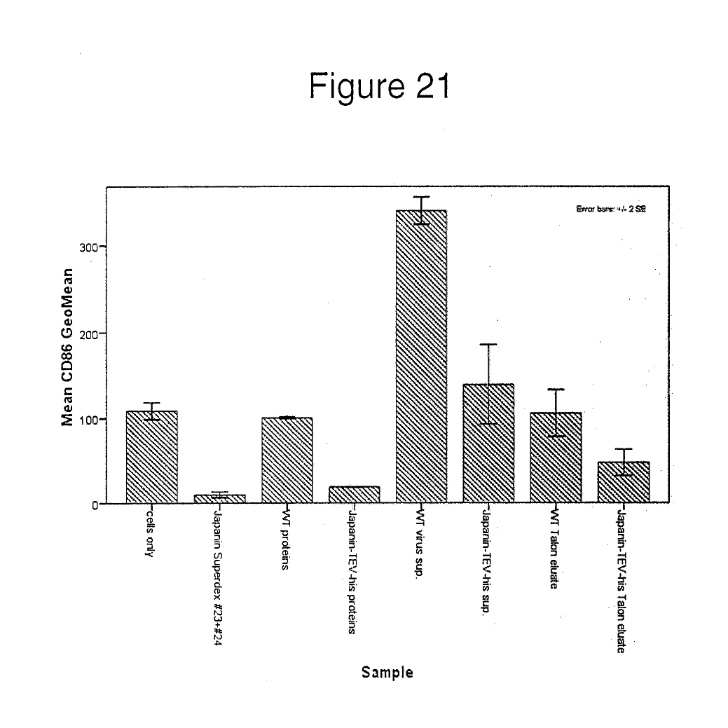

Japanin-TEV-his supernatant-derived samples were screened for DC-modulatory activity as described previously, with the samples being tested at a 1:100 dilution. Superdex fractions #23+24 derived from untagged recombinant Japanin were screened as a positive control for DC-modulatory activity, and samples derived from a Sf9 cell culture infected with wildtype baculovirus served as negative controls. As shown in FIG. 21, activity was present in protein precipitated from Japanin-TEV-his supernatant using ammonium sulphate, showing that active recombinant fusion protein has been produced. Activity was also present in 10× concentrated Talon column-binding proteins from Japanin-TEV-his supernatant, confirming that the active protein does indeed bind Talon resin. The lower activity of the 10× concentrated Talon eluate as compared to the bulk ammonium sulphate-precipitated proteins does not necessarily indicate a reduction in activity, but may instead reflect a stimulatory effect of the Talon elution buffer.

### Example 18

### Purification of His-Tagged Japanin

Polyhistidine-tagged japanin was further purified from Talon column eluate by passing it through a Superdex 75 (gel filtration) column. Fractions containing japanin were identified by the presence of a ˜20 kDa band on a silver-stained SDS-PAGE gel and pooled. The concentration of protein in the pooled fractions was calculated from the absorbance at 280 nm and the extinction coefficient predicted from the mature japanin sequence by the ProtParam tool at expasy.org.

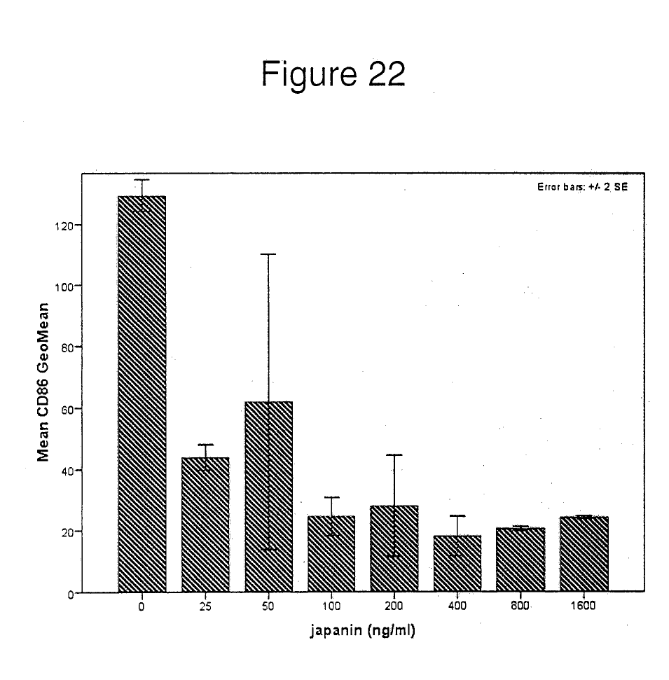

The purified protein was assayed for DC-modulatory activity as described previously at a variety of concentrations, from 25 ng/ml to 1.6 μg/ml, with poly(I:C) added to the cells after 24 hours. FIG. 22 shows that maximal activity was reached at concentrations of 100 ng/ml and above, with the single experiment performed so far suggesting that 50% activity may be reached with <25 ng/ml.

### Example 19

### Effect of Japanin on DC Differentiation

DCs were generated from human monocytes by culture with GM-CSF and IL-4, as previously described. Some of the cultures were additionally supplemented with 200 ng/ml recombinant japanin. The cultures were analysed for CD14 and CD1a expression daily from day 3 to day 6.

It could be argued that any effects of japanin on differentiation were due to endotoxin contamination of the recombinant japanin, rather than to the effects of japanin itself, and so the endotoxin content of the japanin was assessed using the LAL assay, and was found to be ˜0.540EU/μg (approximately equivalent to 0.054 ng E. coli LPS per μg of japanin).

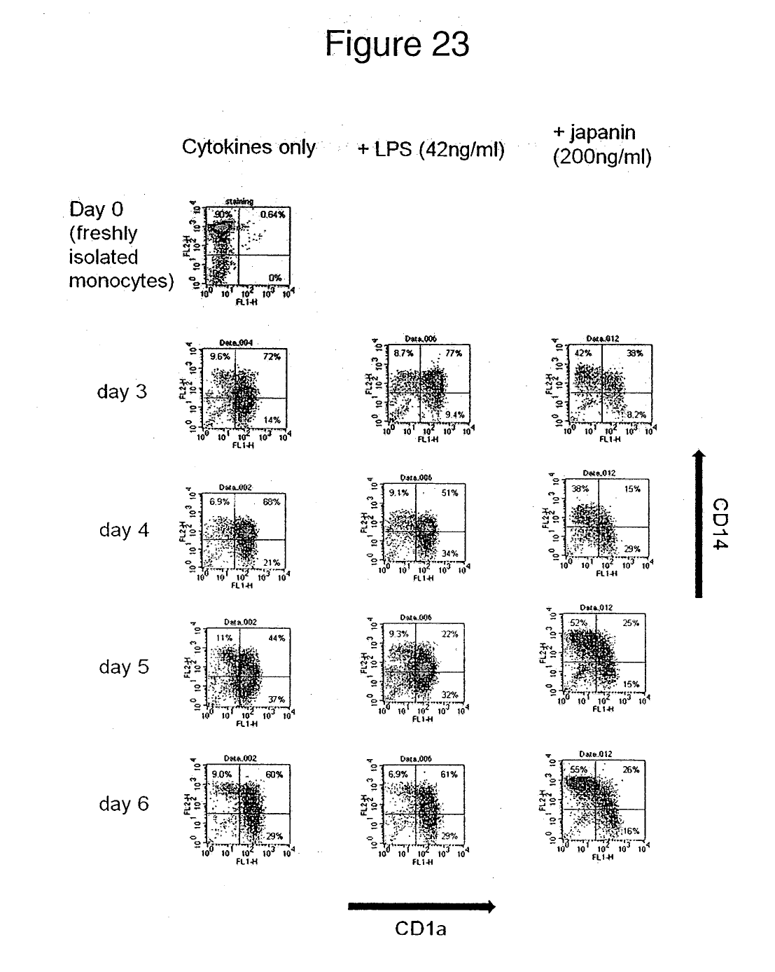

As can be seen from FIG. 23, 200 ng/ml japanin greatly altered the development of the differentiation cultures, with ˜50% of monocytes failing to upregulate CD1a and downregulate CD14, a signature of differentiation into DCs. That this was not a side effect of endotoxin contamination of the japanin was shown by controls in which either 9 pg/ml or 40 ng/ml E. coli LPS (approximately equivalent to the endotoxin content of the recombinant japanin when used at 200 ng/ml, and >4000 times its endotoxin content, respectively) was added, and neither concentration had any major impact on differentiation.

### Example 20

### T Cell Proliferation Assay—Mixed Leucocyte Repsonse (MLR)

A Mixed Leucocyte Response (MLR) was used to assess the effect of japanin on T cell proliferation in response to moDC presenting specific antigens (in this case, allogenic MHC). Japanin was shown to markedly inhibit T cell proliferation in this system.

Frozen day 5 monocyte-derived DC prepared as described above were thawed and cultured with or without 200 ng/ml recombinant japanin for a further 2 days.

Allogenic T cells were isolated from a Buffy coat using CD3 MACS microbeads (Miltenyi Biotech) in accordance with the manufacturer's instructions, with initial fractionation of PBMC being performed with Lymphoprep, as previously described.

1×10⁵ T cells/well were placed into a round-bottomed 96 well plate (Corning), along with graded dilutions of irradiated dendritic cells. The culture media was RPMI 1640 supplemented with 10% FCS+2 mM L-glutamine, 100 U/ml penicillin and 100 μg/ml streptomycin, with a final volume of 200 μl/well. The wells containing DC which had been incubated for two days with japanin were further supplemented with 200 ng/ml japanin. Controls containing T cells without DC were included.

MLR cultures were incubated for 4 days, then pulsed with 0.5 μCi/well ³H-thymidine. They were then incubated for an additional 16-18 hours before harvesting on to glass fibre filters using an automated cell harvester, and subsequent quantification of filter-bound radioactive DNA with a scintillation counter.

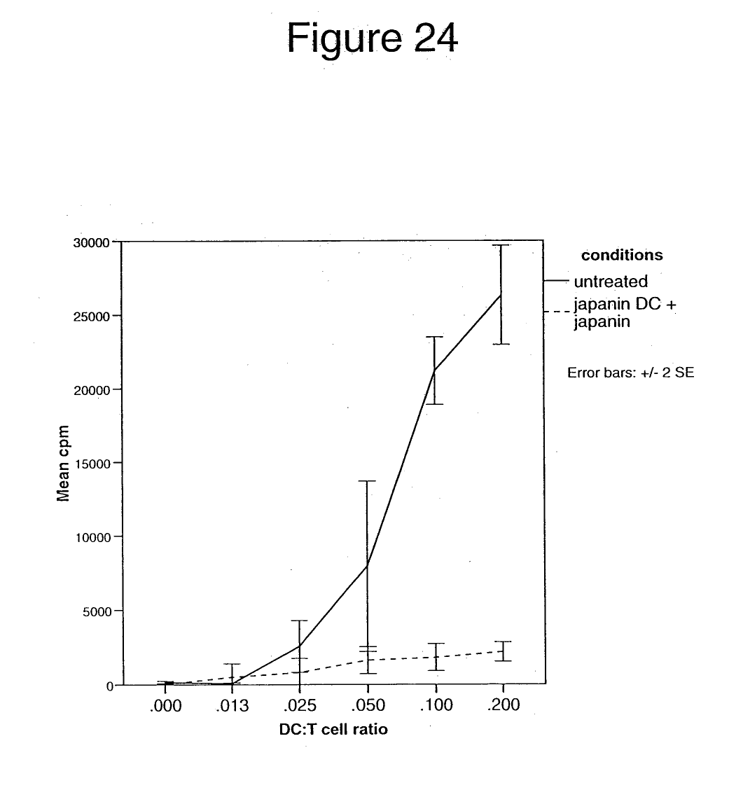

This experiment was performed three times, and each time the pre-treatment of DC with japanin, combined with the presence of japanin in the MLR cultures, was found to reduce T cell proliferation. An example result is shown in FIG. 24.

### Example 21

### Search for Japanin Homologues (Dermacentor andersoni)

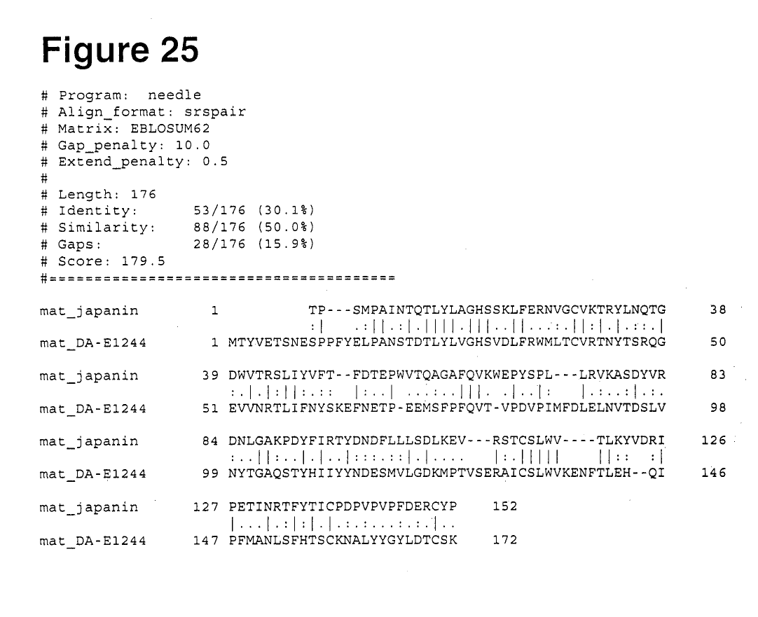

BLAST search of the EMBL Expressed Sequence Tag (EST) database identified a japanin homologue from Dermacentor andersoni. This homologue is currently designated D. andersoni E1244 (EBI ID =EG363153). SignalP was used to identify a likely signal peptide portion of DA-E1244 (residues 1-17), allowing comparison of the mature japanin sequence with the predicted mature DA-E1244 sequence. EMBOSS pairwise alignment, performed using a BLOSUM62 matrix, reports a 30.5% identity and 50.3% similarity between the two proteins, as shown in FIG. 25. The nucleotide sequence present in the database is included as Seq ID no: 32, and the putative encoded protein as Seq ID no: 4.

This high level of homology with japanin strongly suggests that DA-E1244 will possess japanin-like biological activity, and so work is underway to produce recombinant protein.

To this end, DNA coding for the full amino acid sequence of DA-E1244 was produced as a synthetic gene (Seq ID no:3), supplied in a cloning vector, designated pCR4TOPO-DA-E1244_opt (this was done to contract by Entelechon GmbH, Regensburg). The DNA sequence synthesised began with the ATG start codon, and so did not incorporate an upstream sequence matching the Kozak consensus, an essential prerequisite for efficient translation in eukaryotic systems. In order to remedy this, we designed primers which amplified DA-1244 and also added a BamHI recognition site (“GGATCC”) and the Kozak-compliant sequence “TCCAAA” to the 5′ end of the product, and a NotI recognition site (“GCGGCCGC”) to the 3′ end. “Excess” bases were added at both the 5′ and 3′ ends so that the restriction enzyme sites were not at the end of the product, as this is known to inhibit restriction.

Forward primer 8
(SEQ ID NO: 27)
GCAGGCATAGGATCCAAAATGAAACTAAACTTT
Reverse primer 8
(SEQ ID NO: 28)
TATTGCGGCCGCTTATTTCGAACACGT

PCR was performed in a 20 μl reaction using Phusion HotStart DNA polymerase (New England Biosciences) in 1×HF buffer (New England Biosciences) supplemented with 50 μM each dNTP (Bioline) and 250 nM each primer. The pCR4TOPO-DA-E1244_opt plasmid was used as a template, and present in the reaction at 1 ng/gl. An initial denaturation step of 30s at 98° C. was followed by 15 cycles of 10s at 98° C./30s at 69° C./15s at 72° C., and then by an additional 5 minutes at 72° C.

The presence of product of the expected size (600 bp) was confirmed by running 5 μl of the completed reaction on an agarose gel, and the remainder cleaned-up using a QIAquick column (Qiagen) in accordance with the manufacturer's instructions, digested with BamHI and NotI (both from NEB) in Buffer BamHI (NEB), then again cleaned-up with a QIAquick column.

The cut and cleaned DA-1244 PCR product was then ligated into BamHI/NotI digested and Calf Intestinal Phosphatase-treated pBacPAK8 (baculoviral transfer vector), and the ligation reaction used to transform competent TOP10 strain E. coli.

Isolated colonies of transformed E. coli were then picked and grown in liquid culture, and DNA extracted using the QIAprep miniprep kit. Sequencing (using the Bac1 and Bac2 primers) was performed in order to confirm that DA-E1244 had been successful cloned, without the introduction of mutations.

A suitable clone has been identified (pBacPAK8-E1244), and has been employed in the generation of recombinant baculovirus.

### Example 22

### Search for Further Japanin Homologues

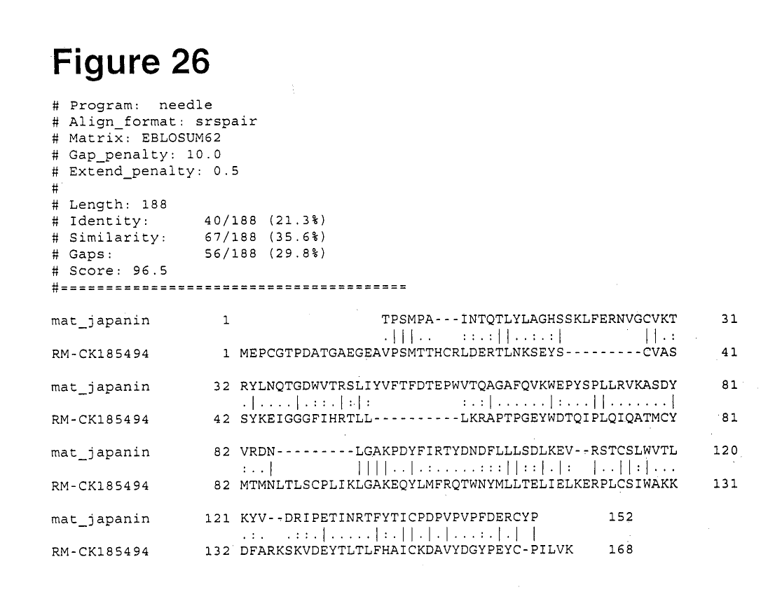

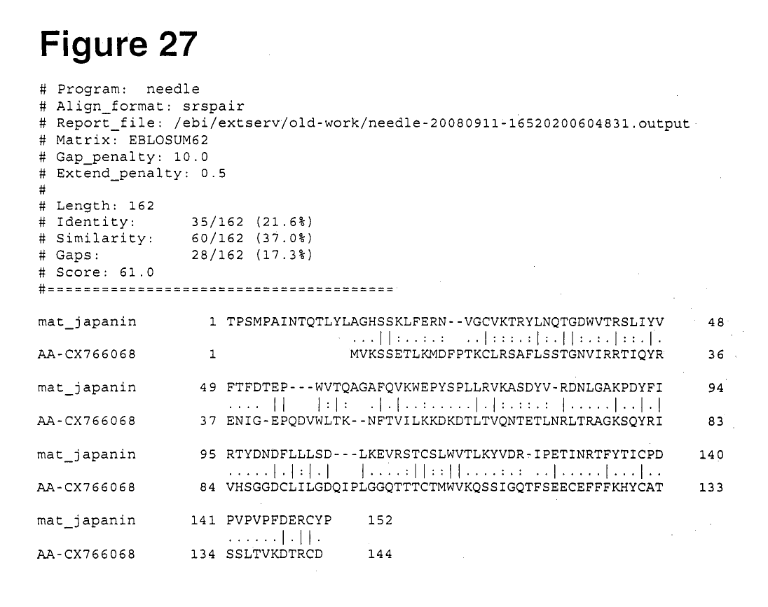

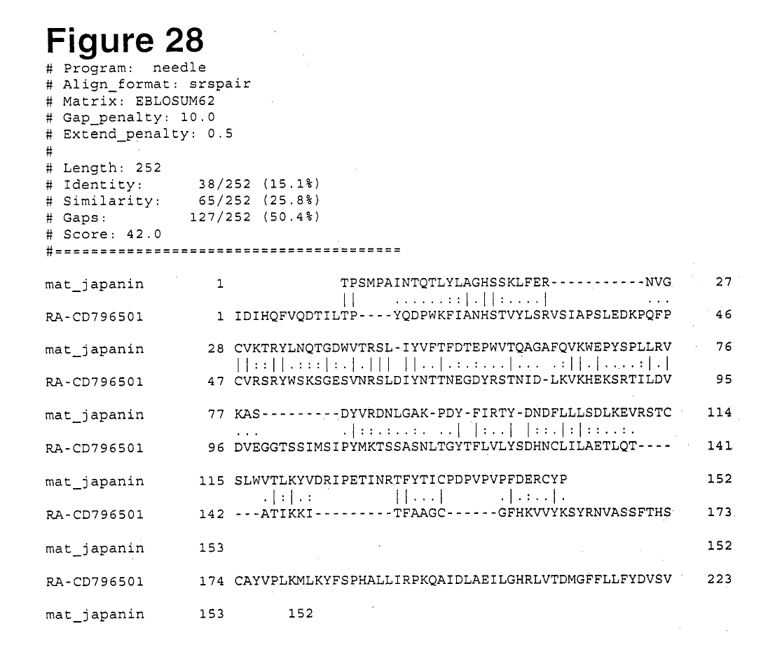

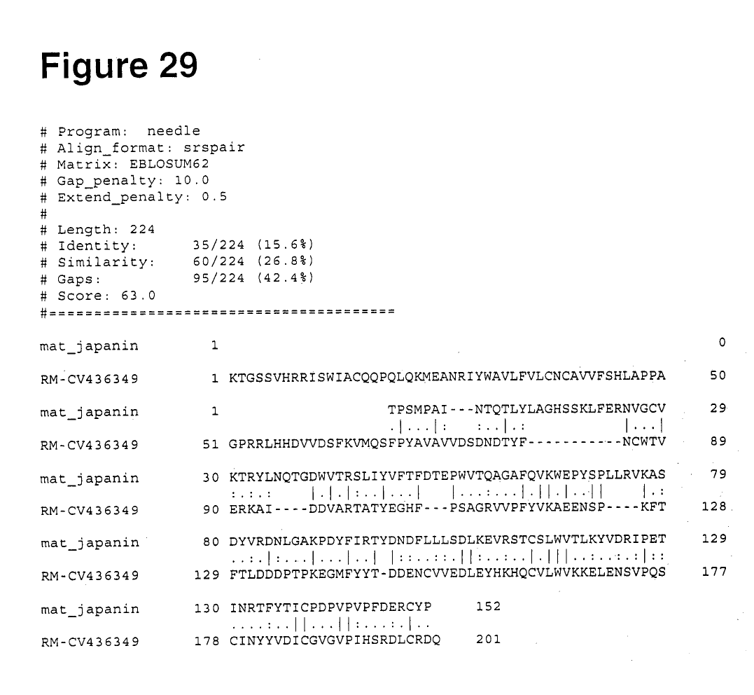

BLAST search of the EMBL Expressed Sequence Tag (EST) database identified japanin homologues from Rhipicephalus microplus (2 homologues), Amblyomma americanum, and Rhipicephalus appendiculatus. These homologues are currently designated R. microplus CK185494, A. americanum CX766068, R. appendiculatus CD796501, and R. microplus CV436349, respectively. The sequence alignments and percentage identity figures for these proteins are shown in FIGS. 26-29.

R. microplus CV436349 is the only one of these identified homologues which contains a signal peptide. There are three possible explanations for the absence of a signal sequence from the three other identified homologues i) they are not secretory proteins; ii) part of the sequence is missing, or iii) non-standard secretion is involved.

R. microplus CK185494 and A. americanum CX766068 are both lipocalins with substantial homology with Japanin. However, neither of these sequences includes a signal sequence, suggesting, as described above, that they are either not secreted proteins, or that part of the sequence is missing.

Although these Japanin homologues have a lower sequence identity with Japanin than the homologue identified from Dermacentor andersoni, their function is expected to be similar to Japanin.

### Example 23

### Identification of Cholesterol as a Japanin Ligand

Japanin has been described as a lipocalin, suggesting the possibility that it may bind a lipid ligand. Construction of a hypothetical structural model of Japanin, based on the crystal structure of OmCI, a fatty acid-binding tick lipocalin, provided additional support for this idea, as it suggested the presence of a hydrophobic, open binding pocket in Japanin (not shown).

In order to investigate this further, recombinant Japanin was produced in insect cell culture as described in Example 13, and purified using sequential metal affinity chromatography and gel filtration, as previously described. 400 μg of this purified recombinant protein were used for gas chromatography-mass spectrometry (GC-MS) analysis, following lipid extraction using the Bligh and Dyer Method, as described below.

Bligh and Dyer Method Lipid Extraction.

3.75 ml of chloroform:methanol (1:2) was added to 0.5 ml of protein sample (or to 0.5 ml of buffer control). This mixture was shaken for 10-15 minutes, then another 1.25 ml of chloroform was added, and mixed in by vortexing for 1 minute. 1.25 ml of ultrapure water was then added, followed by a further 1 minute of vortexing. The resulting sample was centrifuged, and the upper phase discarded, leaving the lower, lipid-containing phase. This was dried under nitrogen and resuspended in 500 μl of dichloromethane.

Gas Chromatography/Electron Impact-Mass Spectrometry (GC/EI-MS)

1 μl of the sample extracted from recombinant japanin or from the buffer blank was injected into a Perkin Elmer Turbomass quadrupole mass spectrometer with integrated capillary gas chromatograph. The following conditions were used:.

Gas chromatography: Column=DB-5. Injection=On-column. Injection Temperature=40° C. Temperature Gradient=40° C. for 1 minute then 8° C./minute to 325° C. (hold for 10 minutes). Carrier Gas=Helium.

Mass Spectrometry: Ionisation Voltage=70 eV. Ionisation Mode=Scanning. MS Resolution=Unit.

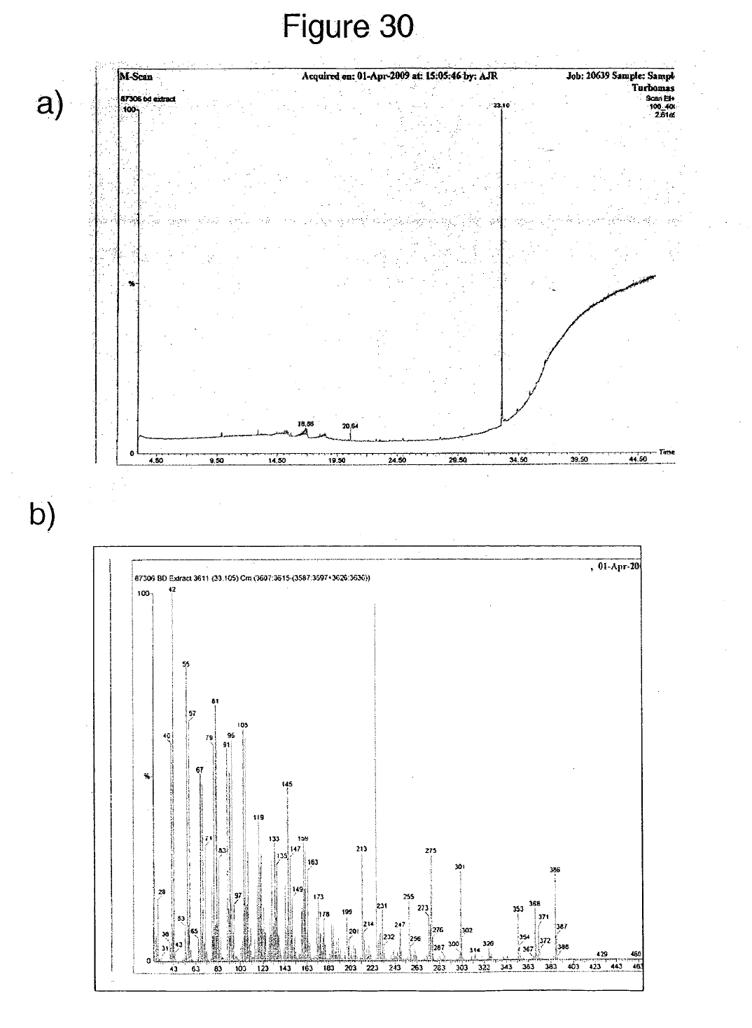

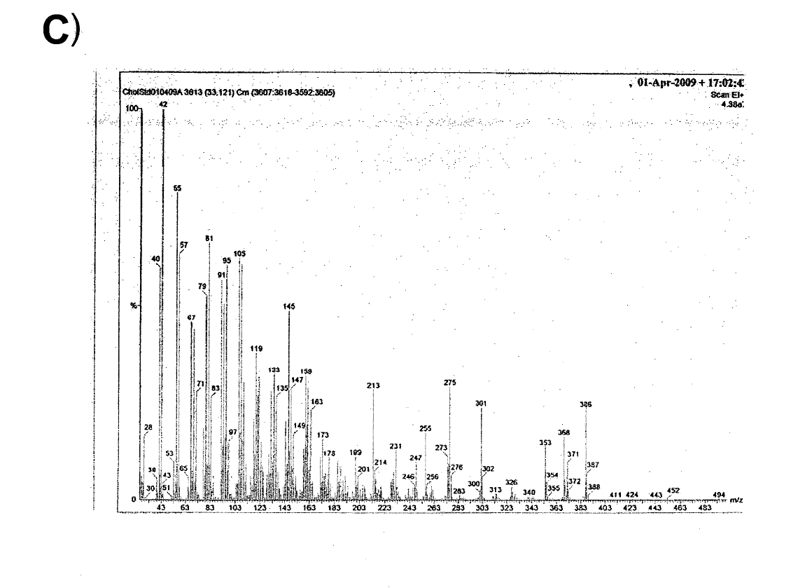

The data obtained from the mass spectrometry showed a peak at 33.1 minutes in the Japanin sample which was not present in the buffer blank (FIG. 30a). Comparison of the averaged spectra from this peak (FIG. 30b) with NIST library spectra allowed its identification as cholesterol. This was confirmed by processing a reference standard of cholesterol under the same conditions, which resulted in a 33.1 minute peak, with matching averaged spectra (FIG. 30c).

### Example 24

### Recombinant Japanin Binds Free Cholesterol

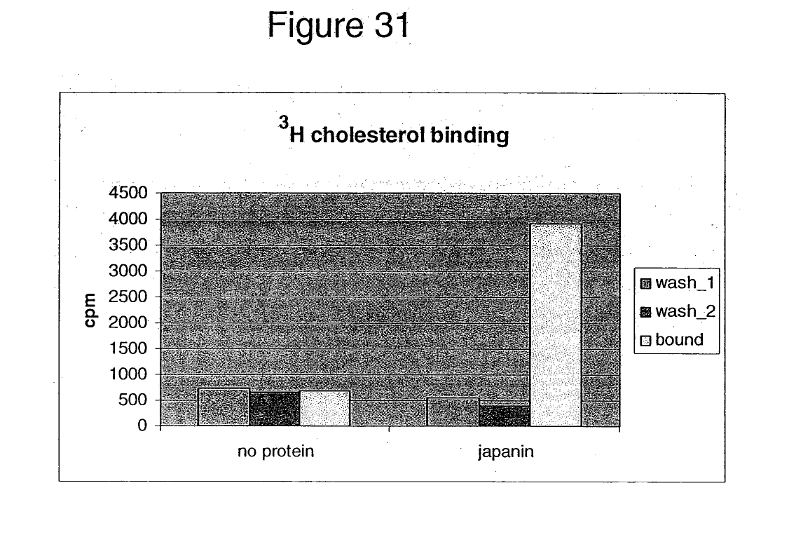

Recombinant, oligohistidine-tagged Japanin was immobilised on Ni-NTA magnetic beads by incubating 0.5 μg of the protein in 500 μl buffer A (120 mM NaCl, 0.02% Tween, 5% glycerol, 40 mM dibasic sodium phosphate) containing the beads for two hours at room temperature. Protein was omitted from the control sample. The protein-coated beads were washed 3 times with 500 μl of buffer A, before adding 50 μl of buffer B (6M guanidine, 120 mM NaCl, 0.02% Tween, 5% glycerol, 40 mM dibasic sodium phosphate) containing 0.2 μl of 3H-cholesterol (the denaturing buffer was used to promote possible exchange of cold, cell culture derived ligand bound to the protein with radiolabelled cholesterol). After 5 minutes, buffer B was removed and 500 μl buffer A containing a further 0.2 μl 3H-cholesterol was added. This was followed by a 3 hour incubation at room temperature. The beads were washed once with 500 μl and twice with 50 μl ice-cold buffer A, to remove unbound cholesterol. Protein was then eluted from the beads by resuspending them in 100 μl buffer A containing imidazole (0.5 M). Wash 1 and 2 in FIG. 31 refer to the 50-ul washes, the right hand bar for each sample shows the amount of radioactivity bound to the beads/protein.

As can be seen in FIG. 31, these results clearly show that 3H-cholesterol binds to Japanin. It is not yet clear if denaturing/refolding is required for protein binding, and the strength and specificity of binding still have to be determined.

### Example 25

### Japanin Binds a C-type Lectin Cell Surface Receptor on Dendritic Cells

The ability of Japanin to inhibit dendritic cell maturation implies its ability to bind to the surface of the dendritic cell. This seems most likely to occur via a membrane receptor-specific interaction with Japanin, but it is also possible to conceive of a mechanism of action by which Japanin binds and enters a cell in a non-specific way, perhaps involving interaction of the bound cholesterol with the plasma membrane, and then acts in a cell type-specific way on intracellular signalling pathways.

In order to investigate whether Japanin binds the surface of dendritic cells in a specific fashion, and to allow the investigation of the nature of any interaction, Japanin was labelled with the fluorescent dye Alexa 488 using a commercial kit. Incubation of cells with 500 ng/ml of this fluorescently-tagged Japanin for 30-60 minutes at 4° C., followed by thorough washing, allowed Japanin binding to be visualised by flow cytometry.

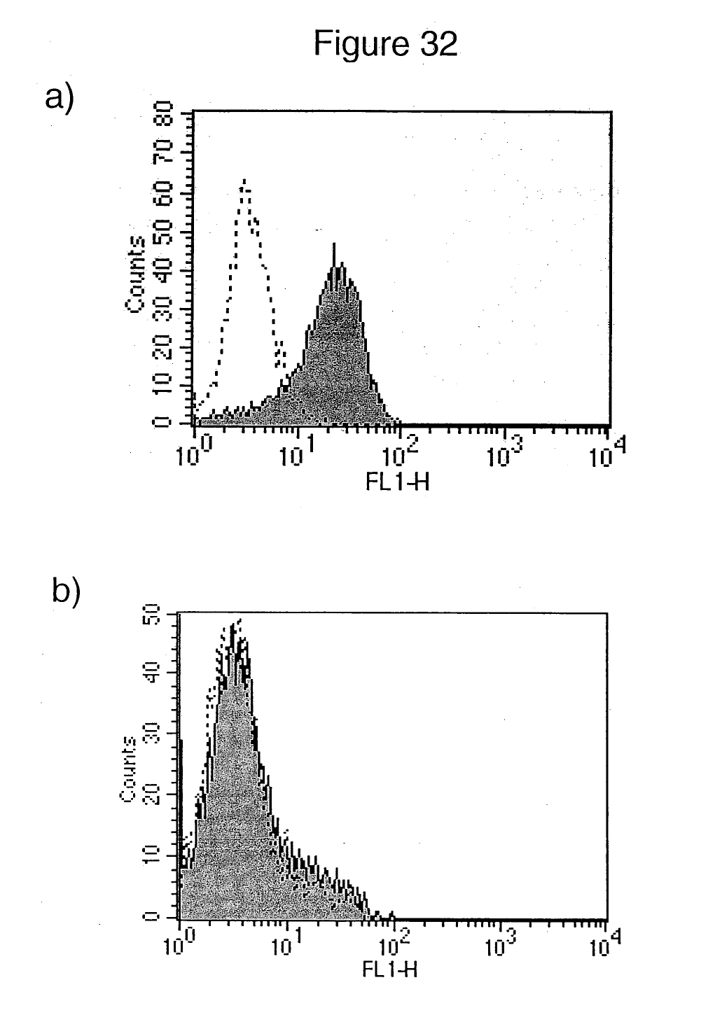

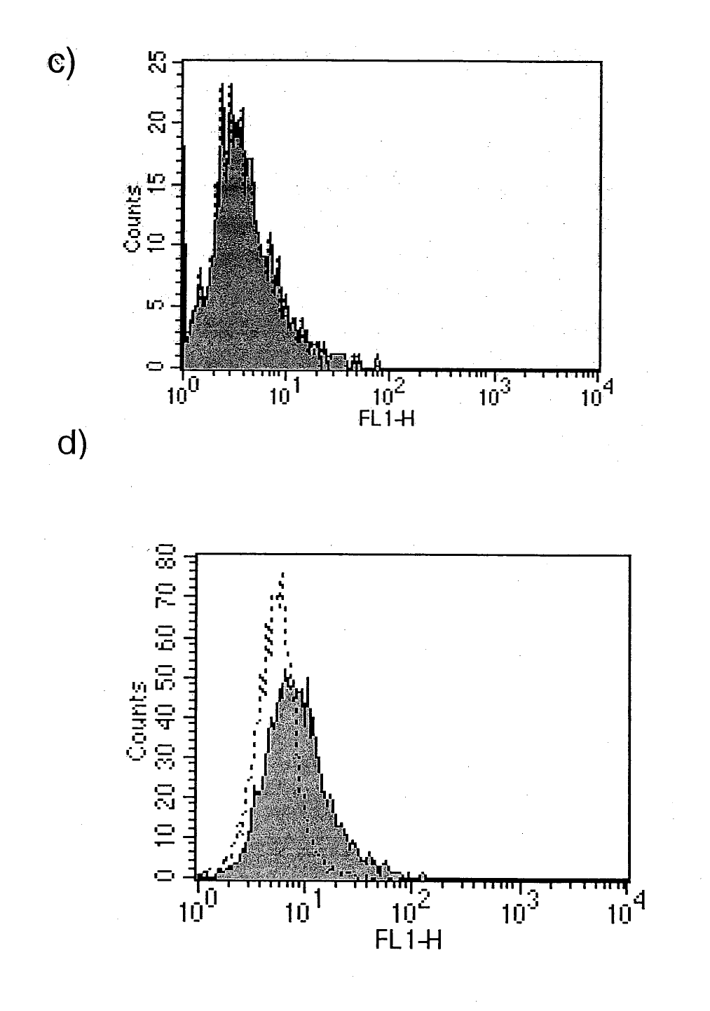

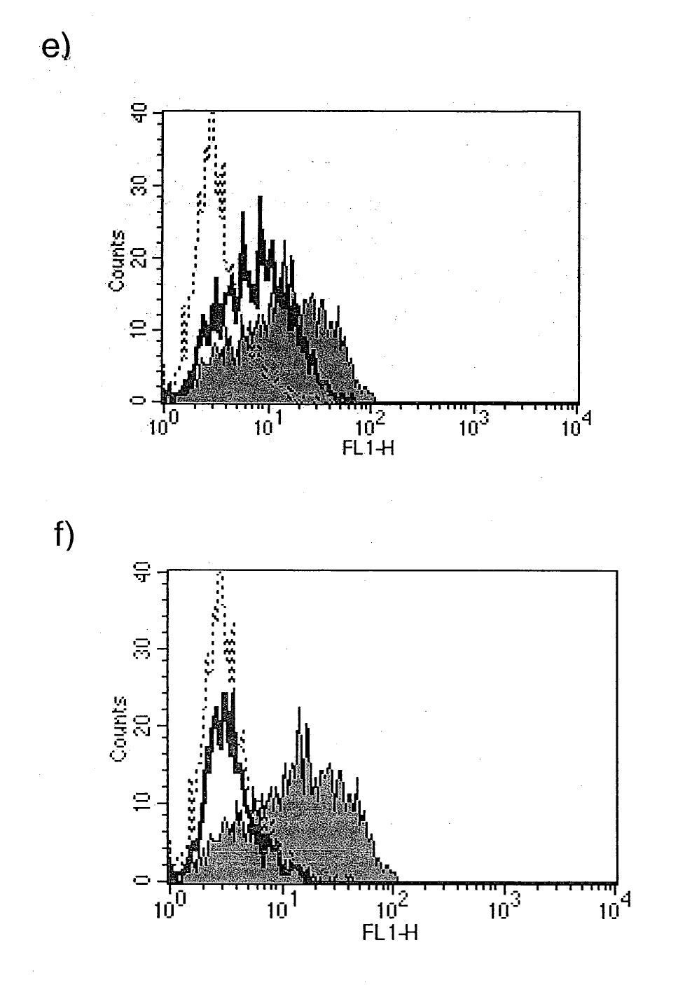

That Japanin specifically binds monocyte-derived dendritic cells is demonstrated by FIG. 32a, which shows that Japanin-Alexa 488 (filled histogram in FIGS. 32a-f) binds to day 5 monocyte-derived dendritic cells (generated as described previously), whereas moubatin-Alexa 488, used as a control lipocalin, does not (dashed-line histogram in FIGS. 32a-f). Furthermore, Japanin does not bind to monocytes (FIG. 32b), nor to mouse bone marrow-derived dendritic cells (FIG. 32c).

The failure of Japanin to bind to monocytes was surprising, given the previously demonstrated ability of Japanin to block monocyte differentiation into dendritic cells. This raises the question of how Japanin is able to act on a cell-type it apparently does not bind to. In order to address this issue, Japanin-Alexa 488 (filled histogram in FIGS. 32a-f) or moubatin-Alexa 488 (dashed-line histogram in FIGS. 32a-f) were incubated with day 1 monocyte-derived dendritic cells (FIG. 32d). Japanin was found to bind to day 1 monocyte-derived dendritic cells, albeit to a lesser extent than to day 5 monocyte-derived dendritic cells. This suggests that the upregulation of the Japanin-binding receptor begins very early in dendritic cell differentiation, and so Japanin may be acting on cells to arrest their differentiation at this early stage.

In order to investigate the nature of the Japanin-dendritic cell interaction, the effects of mannan and EDTA were examined. The presence of 1 mg/ml mannan greatly reduced Japanin-Alexa 488 binding to monocyte-derived dendritic cells (FIG. 32e, filled histogram shows Japanin-Alexa 488 binding in the absence of mannan, open histogram shows binding in the presence of mannan, and dashed-line histogram shows binding of a control protein), whereas the presence of 0.5 mM EDTA completely abolished it (FIG. 32f, as FIG. 32d except that open histogram shows binding in the presence of EDTA). Taken together, these findings strongly suggest that Japanin binds to a C-type lectin cell surface receptor on monocyte-derived dendritic cells.

### Example 26

### Japanin is N-Glycosylated

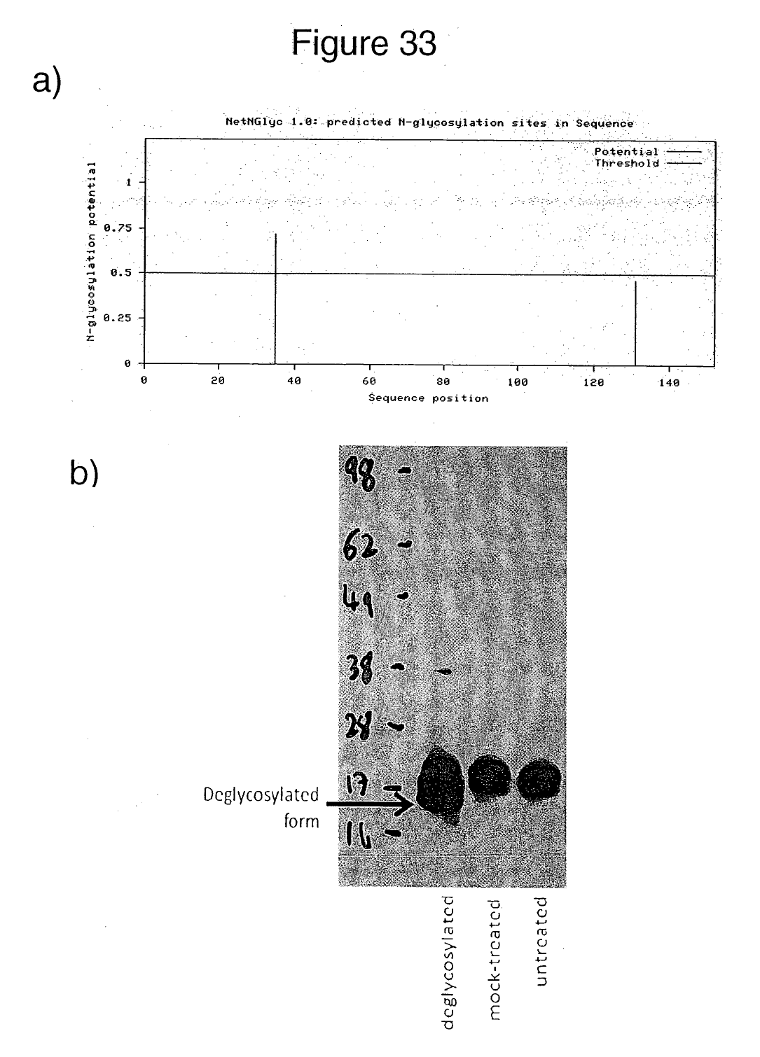

Use of NetNGlyc 1.0 (Center for Biological Sequence Analysis, Technical University of Denmark) suggests that Japanin contains one probable and one other possible N-glycosylation site (FIG. 33a). The presence of some degree of glycosylation is also suggested by the interaction of Japanin with a C-type lectin receptor, as previously described.

In order to confirm the presence of N-glycosylation, purified recombinant Japanin (produced as previously described) was treated for 16 hours at 37° C. with PNGase F, an enzyme which will remove most forms of N-glycosylation. The PNGase F-treated Japanin was then run alongside mock-treated and untreated Japanin on an SDS-PAGE gel, and visualised by anti-his tag Western blot. As shown in FIG. 33b, treatment with PNGase F resulted in the presence of an additional, smaller band in addition to the two bands which comprise mock-treated and untreated Japanin. This demonstrates that at least one, perhaps both, of the larger two bands represent N-glycosylated Japanin.

### Example 27

### Recombinant Japanin Inhibits Dendritic Cell Maturation in Response to Numerous and Diverse Stimuli

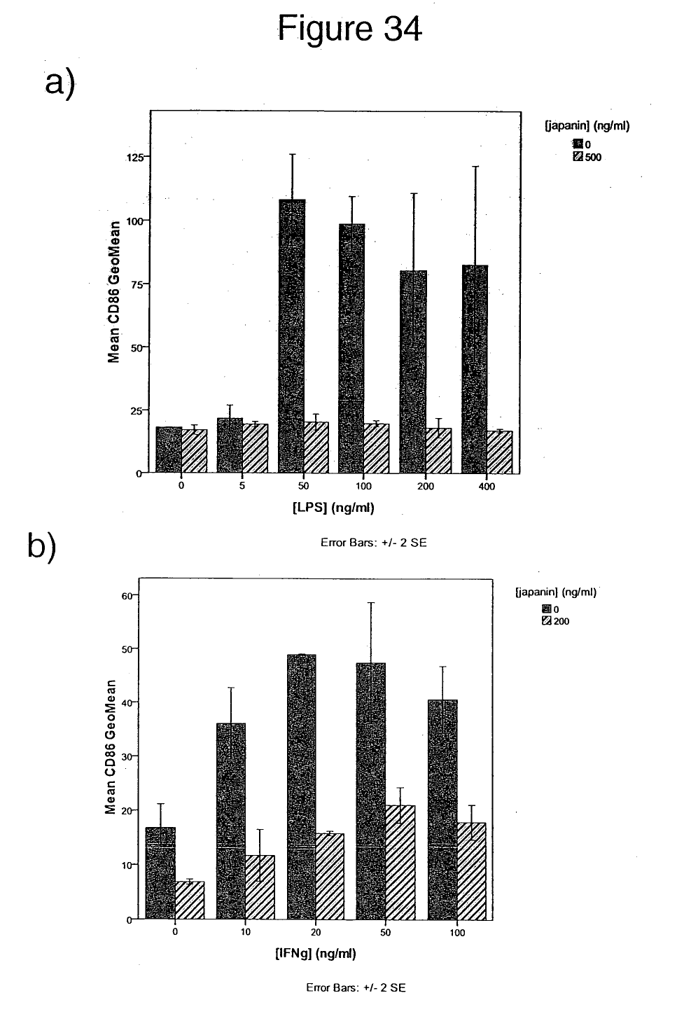

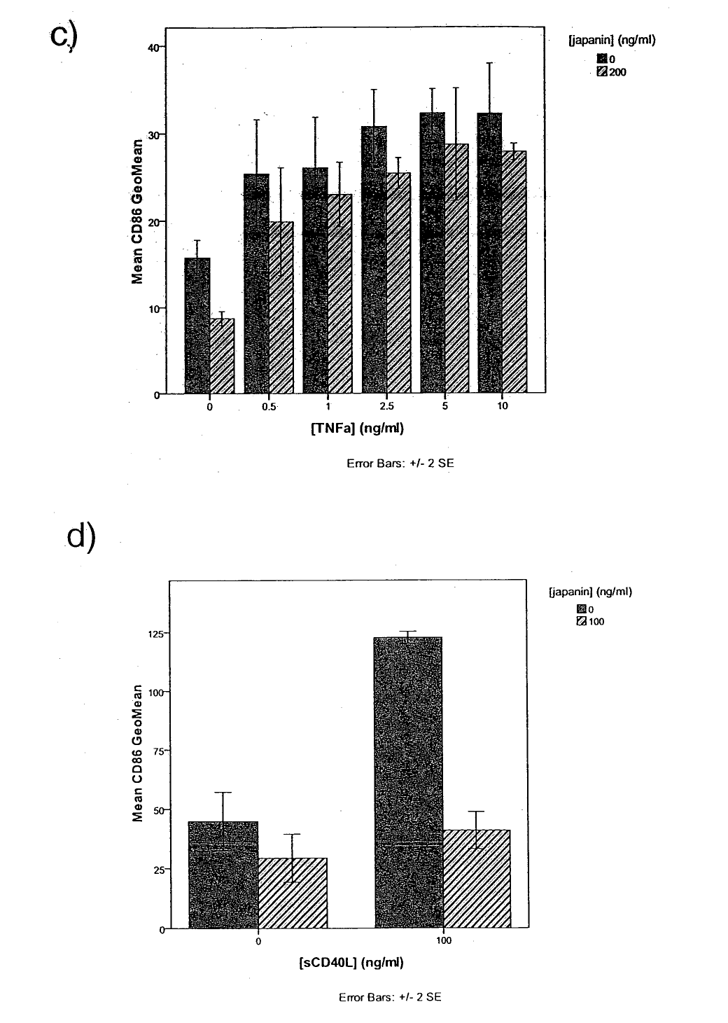

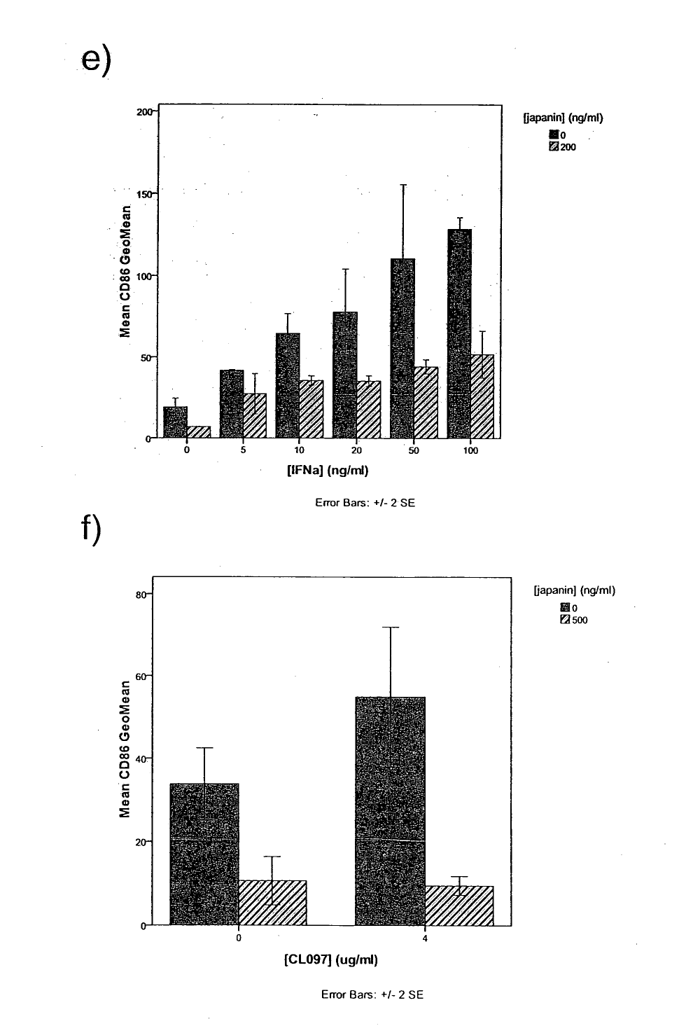

As described previously, recombinant Japanin inhibits monocyte-derived dendritic cell maturation in response to poly(I:C), a TLR3 stimulus, and Japanin-containing Q column flowthrough from 3 day-fed female R. appendiculatus ticks inhibits monocyte-derived dendritic cell maturation in response to LPS, a TLR4 stimuli, and IFNγ, which acts through the γ-interferon receptor, but not to soluble TNFα, which acts through TNFR1. These findings were extended by repeating these experiments (following the same metholology as previously described) using purified recombinant Japanin (produced as previously described) and stimulating with LPS (FIG. 34a), IFNγ (FIG. 34b), TNFα (FIG. 34c), soluble CD40L (FIG. 34d), IFNα (FIG. 34e), or CL097, a TLR7/8 ligand (FIG. 34f). Dendritic cell maturation triggered by all of these stimuli other than TNFα is inhibited by Japanin—no significant effect on TNFα-driven maturation has been observed, though a marginal inhibition may occur. These findings confirm that Japanin is capable of inhibiting dendritic cell maturation in response to a wide range of stimuli, which act through a number of different receptors and downstream signalling pathways.

### Example 28

### Recombinant Japanin Inhibits Dendritic Cell TNFα-Secretion in Response to Stimuli

As well as upregulating co-stimulatory molecules and MHC Class II, dendritic cells also respond to inflammatory stimuli by producing a variety of cytokines. In order to assess whether Japanin was capable of inhibiting or otherwise altering this aspect of dendritic cell maturation, the impact of Japanin on monocyte-derived dendritic cell production of the pro-inflammatory cytokine TNFα in response to a mixture of two stimuli, LPS and IFNγ has been assessed.

Human monocyte-derived dendritic cells were generated as described previously. On day 5 they were harvested and re-suspended in fresh media containing FCS, GCSF and IL4 (as previously described) at a density of 5×10⁵ cells/ml. They were then cultured in 24-well tissue-culture treated plates in the presence or absence of purified recombinant Japanin (500 ng/ml), and after 24 hours a stimuli cocktail of recombinant human IFNγ (Peprotech) and ultrapure E. coli 011:B4 LPS (Alexis Biochemicals) was added to some of the wells, to a final concentration of 20 ng/ml IFNγ+200 ng/ml LPS. After a further 48 hours, the culture supernatants were harvested and centrifuged to remove cells and debris. TNFα concentration was then determined using an ELISA kit (Insight Biotechnology) in accordance with the manufacturer's instructions.

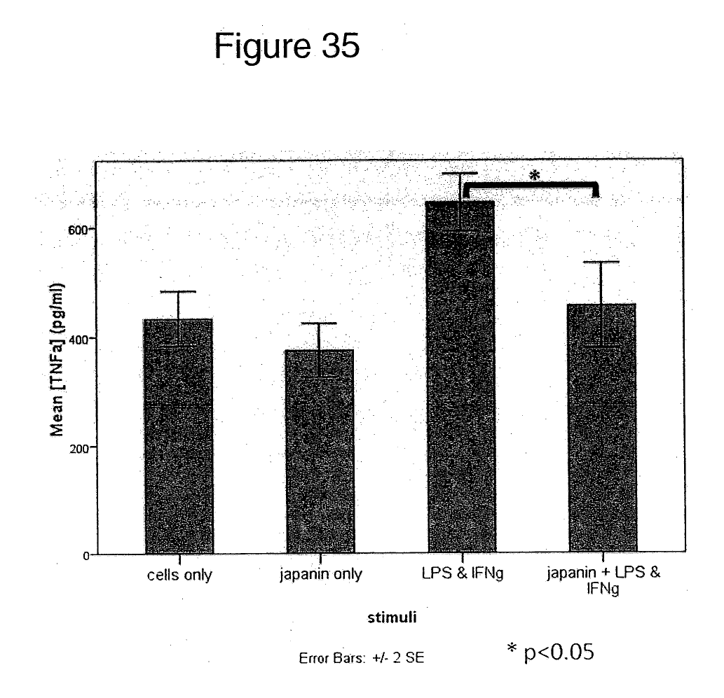

Japanin was found to reduce dendritic cell secretion of TNFα in response to the stimuli cocktail, as shown in FIG. 35.

### REFERENCES

Anguita, J., Ramamoorthi, N., Hovius, J., Das, S., Thomas, V., Persinski, R., Conze, D., Askenase, P., Rincon, M., Kantor, F., Fikrig, E. (2002) Salp15, an Ixodes scapularis salivary protein inhibits CD4⁺ T cell activation Immunity 16, 849-859.
Bergman, D., Palmer, M., Caimano, M., Radolf, J., Wikel, S. (2000) Isolation and molecular cloning of a secreted immunosuppressant protein from Dermacentor andersoni salivary Gland The Journal of Parasitology 86(3), 516-525.
Cavassani, K., Aliberti, J., Dias, A., Silva, J., Ferreira, B. (2005) Tick saliva inhibits differentiation, maturation and function of murine bone-marrow-derived dendritic cells Immunology 114, 235-245.
Coligan et al., Current Protocols in Immunology 1(2):Chapter 5 (1991)
Hannier, S., Liversidge, J., Sternberg, J., Bowman, A. (2004) Characterisation of the B-cell inhibitory protein factor in Ixodes ricinus tick saliva: a potential role in enhanced Borrelia burgdoferi transmission Immunology 113, 401-408.
Hovius, J., Ramamoorthi, N., Veer, C., Groot, K., Nijhof, A., Jongejan, F., Dam, A., Fikrig, E. (2007) Identification of Salp15 homologues in Ixodes ricinus ticks Vector-bourne and Zoonotic Diseases 7(3), 296-302.
Jaworski, D., Jasinskas, A., Metz, C., Bucala, R., Barbour, A. (2001) Identification and characterisation of a homologue of the pro-inflammatory cytokine Macrophage Migration Inhibitory Factor in the tick, Amblyomma americanum Insect Molecular Biology 10(4), 323-331.
Leboulle, G., Crippa, M., Decrem, Y., Mejri, N., Brossard, M., Bollen, A., Godfroid, E. (2002) Characterization of novel salivary immunosuppressive protein from Ixodes ricinus ticks The Journal of Biological Chemistry 88(12), 10083-10089.
Ribeiro J. M. C. (1995) Blood-feeding arthropods: live syringes or invertebrate pharmacologists? Infectious Agents & Disease 4, 143-152.
Sambrook, J. et al, (2000) Molecular Cloning, A Laboratory Manual, Cold Spring Harbor Laboratory Press, Cold Spring Harbor, N.Y.
Sa-Nunes, A., Bafica, A., Lucas, D., Conrads, T., Veenstra, T., Andersen, J., Mather, T., Ribeiro, J., Francischetti, I. (2007) Prostaglandin E₂ is a major inhibitor of dendritic cell maturation and function in Ixodes scapularis saliva The journal of Immunology 179, 1497-1505.
Skallova, A., Iezzi, G., Ampenberger, F., Kopf, M., Kopecky, J. (2008) Tick saliva inhibits dendritic cell migration, maturation, and function while promoting development of Th2 responses Journal of Immunology 180, 6186-6192.
Subklewe, N., Sebelin-Wulf, K., Beier, C., Lietz, A., Mathas, S., Dorken, B., Pezzutto, A. (2007) Dendritic cell maturation stage determines susceptibility to the proteasome inhibitor bortezomib. Human Immunology 68(3), 147-155.
Yu, D., Liang, J., Yu, H., Wu, H., Xu, C., Lu, J., Lai, R. (2006) A tick B-cell inhibitory protein from salivary glands of the hard tick, Hyalomma asiaticum asiaticum Biochemical and Biophysical research communications 343, 585-590

1. A dendritic cell (DC) modulatory molecule, wherein said molecule modulates mammalian DC differentiation and maturation.

2. (canceled)

3. The DC modulatory molecule of claim 1, wherein said molecule modulates or inhibits human DC differentiation and maturation.

4.-9. (canceled)

10. The DC modulatory molecule of claim 1, wherein said molecule is isolated from a tick.

11. The DC modulary molecule of claim 10, wherein the tick is selected from the group Ixodes, Bothriocrotoninae, Amblyomminae, Haemaphysalinae, Rhipicephalinae (including Hyalomminae), Nuttalliellidae, Argasinae, Otobinae, Antricolinae, Nothhoaspinae and Ornithodorinae.

12. The DC modulatory molecule of claim 1, which is a lipocalin.

13. The DC modulatory molecule of claim 12, wherein the molecule is complexed to a lipid.

14. The DC modulatory molecule of claim 13, wherein the lipid is a steroid or a sterol.

15. The DC modulatory molecule of claim 14, wherein the lipid is cholesterol or a metabolite of cholesterol, such as vitamin D3.

16. The DC modulatory molecule of claim 1 which binds to a receptor present on the outer membrane of a DC.

17. The DC modulatory molecule of claim 16, which binds to a C-type lectin receptor.

18. A DC modulatory molecule according to claim 1, wherein said molecule comprises:
i) a protein comprising the amino acid sequence of SEQ ID NO: 2;
ii) a homologue of a protein as defined in i) having at least 60% identity thereto;
iii) an active fragment of a protein as defined in i) above or of a homologue as defined in ii) above; or
iv) a functional equivalent of i), ii) or iii).

19. A DC modulatory molecule according to claim 1, wherein said molecule comprises:
i) a protein comprising the amino acid sequence of any one of SEQ ID NOs: 4, 6, 8, 10 or 12;
ii) a homologue of a protein as defined in i) having at least 60% identity thereto;
iii) an active fragment of a protein as defined in i) above or of a homologue as defined in ii) above; or
iv) a functional equivalent of i), ii) or iii).

20. A nucleic acid molecule comprising a nucleic acid sequence encoding a DC modulatory molecule according to claim 18.

21.-22. (canceled)

23. A vector comprising a nucleic acid sequence of claim 20.

24. A host cell comprising a nucleic acid molecule according to claim 20.

25. (canceled)

26. An antibody which binds to the DC modulatory molecule, according to claim 18.

27. A method of modulating a DC comprising contacting said DC with a DC modulatory molecule of claim 1.

28. (canceled)

29. A pharmaceutical composition comprising the DC modulatory molecule of claim 1, and a pharmaceutically acceptable carrier.

30. The pharmaceutical composition of claim 29 further comprising one or more additional therapeutic agents.

31. The pharmaceutical composition of claim 30, wherein the one or more additional therapeutic agents comprises an anti-inflammatory agent, an immunomodulatory agent, an immunosuppressant, a cytokine, a cytokine mimetic or a cytokine binding protein.

32. The pharmaceutical composition of claim 29, wherein the one or more therapeutic agents comprises a disease-associated element.

33.-36. (canceled)

37. A method of treating an animal suffering from autoimmune disorders, transplant rejection, acute and chronic inflammatory diseases, allergies or hypersensitivity comprising administering to said animal the DC modulatory molecule of claim 1.

38. A method of treating an animal suffering an infectious disease including arthropod-borne diseases comprising administering to said animal the DC modulatory molecule of claim 1.

39. A method of treating an animal suffering from cancer comprising administering to said animal the DC modulatory molecule of claim 1.

40. The method of claims 37, claim 38 or claim 39, wherein the DC modulatory molecule, is administered in combination with a disease associated element.

41. The method of claim 40, wherein the disease associated element is selected from: components associated with infectious agents; allergens; non-self components associated with hypersensitivity reactions other than allergy; self components associated with autoimmune disease; transplantation antigens; and tumour antigens.

42. A method for identifying an agonist or antagonist of the modulatory molecule of claim 1, comprising:
(a) contacting a cell expressing a receptor on its surface with a compound to be screened under conditions to permit binding to the receptor, wherein the receptor is capable of providing a detectable signal in response to the binding of a compound; and
(b) determining whether the compound binds to and activates or inhibits the receptor by measuring the level of a signal generated from the interaction of the compound with the receptor.
